(19) United States
(12) Patent Application Publication
(10) Pub. No.: US 2012/0027727 A1
(43) Pub. Date: Feb. 2, 2012

(54) TARGETED NANOPARTICLES FOR CANCER AND OTHER DISORDERS

(75) Inventors: FREDERICK L. HALL, GLENDALE, CA (US); ERLINDA M. GORDON, GLENDALE, CA (US)

(73) Assignee: Epeius Biotechnologies Corporation, San Marino, CA (US)

(21) Appl. No.: 13/184,458

(22) Filed: Jul. 15, 2011

Related U.S. Application Data
(60) Provisional application No. 61/365,240, filed on Jul. 16, 2010.

(51) Int. Cl. A61K 35/76; A61P 35/00

(52) U.S. Cl. 424/93.2; 424/93.6

(57) ABSTRACT

Targeted gene therapeutic systems are provided for the treatment of cancer, including viral particles. The viral particles are engineered to specifically deliver therapeutic or diagnostic agents to a disease site, such as cancer metastatic sites. Localized dosing regimens are provided to treat diseases such as cancer.

### CROSS REFERENCE

This application claims the benefit of U.S. Provisional Application No. 61/365,240, filed Jul. 16, 2010, which is incorporated herein by reference in its entirety.

### TECHNICAL FIELD

The present disclosure relates generally to methods and compositions for treating cancer. Further, the disclosure relates to methods and systems for administering therapeutically effective vectors.

### BACKGROUND OF THE INVENTION

Proliferative diseases, such as cancer, pose a serious challenge to society. Cancerous growths, including malignant cancerous growths, possess unique characteristics such as uncontrollable cell proliferation resulting in, for example, unregulated growth of malignant tissue, an ability to invade local and even remote tissues, lack of differentiation, lack of detectable symptoms and most significantly, the lack of effective therapy and prevention.

Cancer can develop in any tissue of any organ at any age. The etiology of cancer is not clearly defined but mechanisms such as genetic susceptibility, chromosome breakage disorders, viruses, environmental factors and immunologic disorders have all been linked to a malignant cell growth and transformation. Cancer encompasses a large category of medical conditions, affecting millions of individuals worldwide. Cancer cells can arise in almost any organ and/or tissue of the body. Worldwide, more than 10 million people are diagnosed with cancer every year and it is estimated that this number will grow to 15 million new cases every year by 2020. Cancer causes six million deaths every year or 12% of the deaths worldwide.

Currently, some of the main treatments available are surgery, radiation therapy, chemotherapy and gene therapy. Surgical procedures to treat pancreatic and hepatic cancer may result in partial or total removal of the cancerous organ itself and carries significant risks. Serious adverse effects, including loss of organ function, occurs in cancer-resected patients.

### SUMMARY OF THE INVENTION

This disclosure relates to the administration of targeted viral-based and non-viral particles, including retroviral-based vector particles, adenoviral vector particles, adeno-associated virus vector particles, Herpes Virus vector particles, and pseudotyped viruses such as with the vesicular stomatitis virus G-protein (VSV-G), and to non-viral vectors that contain a viral protein as part of a virosome or other proteoliposomal gene transfer vector. Also provided are retroviral-based expression systems for the generation of targeted therapeutic retroviral particles, the use of transiently transfected human producer cells to produce the particles, a manufacturing process for large scale production of the viral particles, and methods for collecting and storing targeted delivery vectors. Additionally provided are methods for administration of the targeted therapeutic retroviral particles for the treatment of cancer and other disorders, including to halt tumor progression and control tumor growth, to induce remission, to enable surgical resection or to prevent recurrence of the cancer or other disorder. The methods described herein are especially useful in cancers or other disorders that are resistant to traditional therapies, e.g. resistant to chemotherapy, antibody-based therapies or other standard therapies.

In one embodiment, a method for treating cancer in a subject in need thereof with a targeted therapeutic retroviral particle is provided, the method comprising systemically administering a first therapeutic course of at least 1×10¹¹ cfu of a targeted therapeutic retroviral particle, administering via hepatic arterial infusion a second therapeutic course of at least 1×10¹¹ cfu of a targeted therapeutic retroviral particle to the subject; and monitoring the subject for improvement of cancer symptoms.

In one embodiment, the method further comprises a third therapeutic course of at least 1×10¹² cfu of targeted therapeutic retroviral particles following administration via hepatic arterial infusion of a second therapeutic course of at least 1×10¹¹ cfu of a targeted therapeutic retroviral particle to the subject.

In some embodiments, the first and/or second therapeutic course comprises treatment with the targeted therapeutic retroviral particles for at least three days. In other embodiments, the first and/or second therapeutic course comprises treatment with the targeted therapeutic retroviral particle for at least five days. In yet other embodiments, the first and/or second therapeutic course comprises treatment with the targeted therapeutic retroviral particles for at least one week. In still other embodiments, the first and/or second therapeutic course comprises treatment with the targeted therapeutic retroviral particles for at least two weeks. In yet another embodiment, the first and/or second therapeutic course comprises treatment with the targeted therapeutic retroviral particles for at least three weeks. In one embodiment, the first therapeutic course comprises treatment with the targeted therapeutic retroviral particles for at least one week, followed by the second therapeutic course with the targeted therapeutic retroviral particle for at least three days. In still another embodiment, the first therapeutic course comprises treatment with the targeted therapeutic retroviral particles for at least one week, followed by the second therapeutic course with the targeted therapeutic retroviral particle for at least one week. In yet other embodiments, the first therapeutic course comprises treatment with the targeted therapeutic retroviral particles for at least two weeks, followed by the second therapeutic course with the targeted therapeutic retroviral particle for at least one week.

In some embodiments, the first and/or second therapeutic course is administered intravenously. In other embodiments, the first and/or second therapeutic course is administered via intra-arterial infusion, including but not limited to infusion through the hepatic artery, cerebral artery, coronary artery, pulmonary artery, iliac artery, celiac trunk, gastric artery, splenic artery, renal artery, gonadal artery, subclavian artery, vertebral artery, axilary artery, brachial artery, radial artery, ulnar artery, carotid artery, femoral artery, inferior mesenteric artery and/or superior mesenteric artery. Intra-arterial infusion may be accomplished using endovascular procedures, percutaneous procedures or open surgical approaches. In some embodiments, the first and second therapeutic course may be administered sequentially. In yet other embodiments, the first and second therapeutic course may be administered simultaneously. In still other embodiments, the optional third therapeutic course may be administered sequentially or simultaneously with the first and second therapeutic courses.

In one embodiment, the subject is allowed to rest 1 to 2 days between the first therapeutic course and second therapeutic course. In some embodiments, the subject is allowed to rest 2 to 4 days between the first therapeutic course and second therapeutic course. In other embodiments, the subject is allowed to rest at least 2 days between the first and second therapeutic course. In yet other embodiments, the subject is allowed to rest at least 4 days between the first and second therapeutic course. In still other embodiments, the subject is allowed to rest at least 6 days between the first and second therapeutic course. In some embodiments, the subject is allowed to rest at least 1 week between the first and second therapeutic course. In yet other embodiments, the subject is allowed to rest at least 2 weeks between the first and second therapeutic course. In one embodiment, the subject is allowed to rest at least one month between the first and second therapeutic course. In some embodiments, the subject is allowed to rest at least 1-7 days between the second therapeutic course and the optional third therapeutic course. In yet other embodiments, the subject is allowed to rest at least 1-2 weeks between the second therapeutic course and the optional third therapeutic course.

In another embodiment, the first and/or second therapeutic course comprises administration of the targeted therapeutic retroviral particles topically, intravenously, intra-arterially, intracolonically, intratracheally, intraperitoneally, intranasally, intravascularly, intrathecally, intracranially, intramarrowly, intrapleurally, intradermally, subcutaneously, intramuscularly, intraocularly, intraosseously and/or intrasynovially. In still other embodiments, the first and/or second therapeutic course comprises administration of the targeted therapeutic retroviral particles intravenously. In yet other embodiments, the first and/or second therapeutic course comprises administration via intra-arterial infusion. In some embodiments, the optional third therapeutic course may be administered topically, intravenously, intra-arterially, intracolonically, intratracheally, intraperitoneally, intranasally, intravascularly, intrathecally, intracranially, intramarrowly, intrapleurally, intradermally, subcutaneously, intramuscularly, intraocularly, intraosseously and/or intrasynovially.

In some embodiments, the cancer being treated is selected from the group consisting of breast cancer, skin cancer, bone cancer, prostate cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, melanoma, osteosarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythemia vera, adenocarcinoma, glioblastoma multiforma, leukemias, lymphomas, malignant melanomas, and epidermoid carcinomas. In other embodiments, the cancer being treated is pancreatic cancer, liver cancer, breast cancer, osteosarcoma, lung cancer, soft tissue sarcoma, cancer of the larynx, melanoma, ovarian cancer, brain cancer, Ewing's sarcoma or colon cancer.

In one embodiment, the targeted therapeutic retroviral particle accumulates in the subject in areas of exposed collagen. In some embodiments, the areas of exposed collagen include neoplastic lesions, areas of active angiogenesis, neoplastic lesions, areas of vascular injury, surgical sites, inflammatory sites and areas of tissue destruction. In yet other embodiments, the targeted therapeutic retroviral particle is a retroviral vector having an envelope protein modified to contain a collagen binding domain, and encodes a therapeutic agent against the cancer. In still another embodiment, the retroviral vector is amphotropic. In other embodiments, the therapeutic agent is a cyclin G1 mutant. In still other embodiments, the therapeutic agent is an N-terminal deletion mutant of cyclin G1. In some embodiments, the N-terminal deletion mutant of cyclin G1 comprises from about amino acid 41 to 249 of human cyclin G1. In other embodiments the therapeutic agent is interleukin-2 (IL-2). In yet other embodiments, the therapeutic agent is granulocyte macrophage-colony stimulating factor (GM-CSF). In still other embodiments, the therapeutic agent is thymidine kinase.

In another embodiment, a method for producing a targeted therapeutic retroviral particle is provided. The method includes transiently transfecting a producer cell with 1) a first plasmid comprising a nucleic acid sequence encoding the 4070A amphotropic envelope protein modified to contain a collagen binding domain; 2) a second plasmid comprising i) a nucleic acid sequence operably linked to a promoter, wherein the sequence encodes a viral gag-pol polypeptide; ii) a nucleic acid sequence operably linked to a promoter, wherein the sequence encodes a polypeptide that confers drug resistance on the producer cell; and iii) an SV40 origin of replication; 3) a third plasmid comprising i) a heterologous nucleic acid sequence operably linked to a promoter, wherein the sequence encodes a diagnostic or therapeutic polypeptide; ii) 5′ and 3′ long terminal repeat sequences; iii) a Ψ retroviral packaging sequence; iv) a CMV promoter upstream of the 5′ LTR; v) a nucleic acid sequence operably linked to a promoter, wherein the sequence encodes a polypeptide that confers drug resistance on the producer cell; vi) an SV40 origin of replication. The producer cell is a human cell that expresses SV40 large T antigen. In ones aspect, the producer cell is a 293T cell.

In some embodiments, the retroviral vector is produced by a method comprising: a) transiently transfecting a producer cell with: a first plasmid comprising a nucleic acid sequence encoding the 4070A amphotropic envelope protein modified to contain a collagen binding domain, wherein the nucleic acid sequence is operably linked to a promoter; a second plasmid comprising: a nucleic acid sequence operably linked to a promoter, wherein the sequence encodes a viral gag-pol polypeptide, a nucleic acid sequence operably linked to a promoter, wherein the sequence encodes a polypeptide that confers drug resistance on the producer cell, an SV40 origin of replication; a third plasmid comprising: a heterologous nucleic acid sequence operably linked to a promoter, wherein the sequence encodes a diagnostic or therapeutic polypeptide, 5′ and 3′ long terminal repeat sequences (LTRs), a Ψ retroviral packaging sequence, a CMV promoter upstream of the 5′ LTR, a nucleic acid sequence operably linked to a promoter, wherein the sequence encodes a polypeptide that confers drug resistance on the producer cell, an SV40 origin of replication, wherein the producer cell is a human cell that expresses SV40 large T antigen; b) culturing the producer cells of a) under conditions that allow targeted delivery vector production and release in to the supernatant of the culture; and c) collecting the retroviral vectors.

The collected particles generally exhibit a viral titer of about 1×10⁷ to 1×10¹², 1×10⁸ to 1×10¹¹, 1×10⁹ to 1×10¹¹, 5×10⁸ to 5×10¹⁰, or 1×10⁹ to 5×10¹¹, at least 5×10⁸, 1×10⁹, 5×10⁹, 1×10¹⁰, 5×10¹⁰, 1×10¹¹, 1×10¹², 1×10¹³ or 1×10¹⁴ colony forming units per milliliter. In addition, the viral particles are generally about 10 nm to 1000 nm, 20 nm to 500 nm, 50 nm to 300 nm, 50 nm to 200 nm, or 50 nm to 150 nm in diameter.

In one embodiment, the first plasmid is the Bv1/pCAEP plasmid. In another embodiment, the first plasmid is an pB-RVE plasmid. In some embodiments, the second plasmid is the pCgpn plasmid. In one embodiment, the third plasmid is derived from the G1XSvNa plasmid. In yet another embodiment, the third plasmid is the pdnG1/C-REX plasmid. In still another embodiment, the third plasmid is the pdnG1/C-REX II plasmid. In yet another embodiment, the third plasmid is the pdnG1/UBER-REX plasmid.

In some embodiments, the targeted therapeutic retroviral particle comprises a collagen binding domain comprising a peptide derived from the D2 domain of von Willebrand factor. In one embodiment, the von Willebrand factor is bovine von Willebrand factor. In still other embodiments, the peptide comprises the amino acid sequence Gly-His-Val-Gly-Trp-Arg-Glu-Pro-Ser-Phe Met-Ala-Leu-Ser-Ala-Ala (SEQ ID NO:1). In yet another embodiment, the peptide comprises the amino acid sequence Gly-His-Val-Gly-Trp-Arg-Glu-Pro-Ser-Phe-Met-Ala-Lys-Ser-Ala-Ala (SEQ ID NO:2). In some embodiments, the peptide is contained in the gp70 portion of the 4070A amphotropic envelope protein.

In some embodiments, the methods above further comprise administering to the subject a chemotherapeutic agent, a biologic agent, or radiotherapy prior to, contemporaneously with, or subsequent to the administration of the therapeutic viral particles.

In some embodiments, at least one of an abdominal CT scan, MRI, abdominal ultrasound, CBC, platelet count, Chem panel (BUN, Creatinine, AST, ALT, Alk Phos, Bilirubin), electrolytes, PT or PTT measurements is monitored in the subject for improvement of cancer symptoms. In yet other embodiments, tumor lesion(s) is monitored for improvement of cancer symptoms. In one embodiment, the tumor lesion(s) is measured by calipers or by radiologic imaging. In yet other embodiments, the radiologic imaging is MRI, CT, PET, or SPECT scan.

Also provided are methods of treating cancer in a subject in need thereof with a targeted therapeutic retroviral particle, the method comprising: a) systemically administering a first therapeutic course of at least 1×10¹¹ cfu of a targeted therapeutic retroviral particle for at least three days; b) administering via hepatic arterial infusion a second therapeutic course of at least 1×10¹¹ cfu a targeted therapeutic retroviral particle to the subject for at least three days; and c) monitoring the subject for improvement of cancer symptoms. In some embodiments, the methods provided further comprise a third therapeutic course of at least 1×10¹¹ cfu of targeted therapeutic retroviral particles following step b).

Targeted therapeutic retroviral particles disclosed herein generally contain nucleic acid sequences encoding diagnostic or therapeutic polypeptides. As described in greater detail in other portions of this specification, exemplary therapeutic proteins and polypeptides of the invention include, but are in no way limited to, those of the classes of suicidal proteins, apoptosis-inducing proteins, cytokines, interleukins, and TNF family proteins. Exemplary diagnostic proteins or peptides, include for example, a green fluorescent protein and luciferase.

In another embodiment, a plasmid including a multiple cloning site functionally-linked to a promoter, wherein the promoter supports expression of a heterologous nucleic acid sequence; 5′ and 3′ long terminal repeat sequences; a Ψ retroviral packaging sequence; a CMV promoter positioned upstream of the 5′ LTR; a nucleic acid sequence operably linked to a promoter, wherein the sequence encodes a polypeptide that confers drug resistance on a producer cell containing the plasmid; and an SV40 origin of replication. Exemplary plasmids include pC-REX II, pC-REX and pUBER-REX. Additional derivatives of the exemplary include those that contain a heterologous nucleic acid sequence encoding a therapeutic or diagnostic polypeptide.

In another embodiment, a kit for treating cancer is provided. The kit includes a container containing a viral particle produced by a method described herein in a pharmaceutically acceptable carrier and instructions for administering the viral particle to a subject. The administration can be according to the exemplary treatment protocol provided herein.

In another embodiment, a method for conducting a gene therapy business is provided. The method includes generating targeted therapeutic retroviral particles and establishing a bank of the same by harvesting and suspending the therapeutic retroviral particles in a solution of suitable medium and storing the suspension. The method further includes providing the particles, and instructions for use of the particles, to a physician or health care provider for administration to a subject (patient) in need thereof. Such instructions for use of the particles can include the exemplary treatment regimen provided in Table 1. The method optionally includes billing the patient or the patient's insurance provider.

In yet another embodiment, a method for conducting a gene therapy business, including providing kits disclosed herein to a physician or health care provider, is provided.

In other embodiments, the subject is a mammal, preferably a human.

In some embodiments, the therapeutic retroviral particles are inventive viral vectors disclosed here, such as viral vectors which are retroviral (preferably amphotropic) vectors having an envelope protein modified to contain a collagen binding domain, and encodes a therapeutic agent (such a cytocidal mutant of cyclin G1) against the cancer.

In other embodiments, the method may further include the following step: administering to the subject a chemotherapeutic agent, a biologic agent, or radiotherapy prior to, contemporaneously with, or subsequent to the administration of the therapeutic retroviral particles.

These, and other aspects, embodiments, objects and features of the present invention, as well as the best mode of practicing the same, will be more fully appreciated when the following detailed description of the invention is read in conjunction with the accompanying drawings.

### BRIEF DESCRIPTION OF THE DRAWINGS

FIG. 1A depicts a representative MRI from Patient #1 one day after completion of treatment cycle #1 showing a large round recurrent tumor (T; bracketed area) in the region of the pancreas within the area of the surgical bed and an enlarged para-aortic lymph node (N) indicating metastasis.

FIG. 1B depicts a follow-up MRI from Patient #1 four days after completion of treatment cycle #2 showing an irregularity in the shape of the recurrent tumor (T; bracketed area) with a large area of central necrosis (nec) involving 40-50% of the tumor mass, and a significant decrease in the size of the para-aortic lymph node metastasis (N).

FIG. 1C is a graph showing that REXIN-G induces a reduction in CA19-9 serum level in Patient #1. Serum CA19-9 levels (U/ml), plotted on the vertical axis, are expressed as a function of time (date), plotted on the horizontal axis. The start of each treatment cycle is indicated by arrows.

FIG. 2A provides a representative abdominal CT scan from Patient #2 obtained at the beginning of treatment cycle #1 revealing a 6.0 cm3 mass in the region of the pancreatic head (T) encroaching on the superior mesenteric vein (SMV) and the superior mesenteric artery (SMA).

FIG. 2B provides a follow-up abdominal CT scan from Patient #2 two days after completion of treatment cycle #2, revealing that the pancreatic tumor mass (T) has decreased in size and regressed away from the superior mesenteric vessels (SMV and SMA). The start of each treatment cycle is indicated by arrows.

FIG. 2C is a graph showing that REXIN-G arrests primary tumor growth in Patient #2. A progressive decrease in tumor size was noted with successive treatment with REXIN-G. Tumor volume (cm³) derived by using the formula: width²×length×0.52 (O'Reilly et al. Cell 88, 277, 1997), and plotted on the vertical axis, is expressed as a function of time, plotted on the horizontal axis. The start of each treatment cycle is indicated by arrows.

FIG. 3A depicts data indicating REXIN-G plus gemcitabine induces tumor regression in Patient #3 with metastatic pancreatic cancer. Tumor volumes (cm³) of primary tumor is plotted on the Y axis and are expressed as a function of time, date. The start of REXIN-G infusions is indicated by arrows.

FIG. 3B depicts data indicating REXIN-G plus gemcitabine induces tumor regression in Patient #3 with metastatic pancreatic cancer. Tumor volume of portal node is plotted on the Y axis and are expressed as a function of time, date. The start of REXIN-G infusions is indicated by arrows.

FIG. 3C depicts data indicating REXIN-G plus gemcitabine induces tumor regression in Patient #3 with metastatic pancreatic cancer. The number of liver nodules is plotted on the Y axis, are expressed as a function of time, date. The start of REXIN-G infusions is indicated by arrows.

FIG. 4A the systolic blood pressure, expressed as mm Hg, plotted on the vertical axis, while time of REXIN-G infusion is plotted on the horizontal axis, for patient #1.

FIG. 4B pulse rate per minute plotted on the vertical axis, while time of REXIN-G infusion is plotted on the horizontal axis, for patient #1.

FIG. 4C respiratory rate per minute are plotted on the vertical axis, while time of REXIN-G infusion is plotted on the horizontal axis, for patient #1.

FIG. 5A depicts data indicating the hemoglobin (gms %), white blood count and platelet count for patient #1 plotted on the Y axis and expressed as a function of treatment days, plotted on the X axis.

FIG. 5B depicts data indicating that REXIN-G has no adverse effects on for patient #1 liver function. AST (U/L) ALT (U/L), and bilirubin (mg %), plotted on the Y axis, are expressed as a function of treatment days, plotted on the X axis.

FIG. 5C depicts patient #1 Blood urea nitrogen (mg %), creatinine (mg %) and potassium (mmol/L) levels, plotted on the Y axis, expressed as a function of treatment days, plotted on the X axis. Dose Level I (4.5×10⁹ cfu/dose) was given for 6 consecutive days, rest period for two days, followed by Dose Level II (9×10⁹ cfu/dose) for 2 days, and then Dose Level III (1.4×10¹⁰ cfu/dose) for 2 days.

FIG. 6 provides data indicating that dose escalation of REXIN-G has no adverse effects on Patient #2's hemodynamic functions. For each dose level, the systolic blood pressure (mm Hg), pulse rate/min, and respiratory rate/per minute are plotted on the vertical axis as a function of time of infusion, plotted on the horizontal axis.

FIG. 7A depicts hemoglobin (gms %), white blood count and platelet count for patient #2 plotted on the Y axis and expressed as a function of treatment days, plotted on the X axis.

FIG. 7B depicts data indicating that REXIN-G has no adverse effects on for patient #2 liver function. AST (U/L) ALT (U/L), and bilirubin (mg %), plotted on the Y axis, are expressed as a function of treatment days, plotted on the X axis.

FIG. 7C depicts blood urea nitrogen (mg %), creatinine (mg %) and potassium (mmol/L) levels for patient #2, plotted on the Y axis expressed as a function of treatment days, plotted on the X axis. Dose Level I (4.5×10⁹ cfu/dose) was given for 5 consecutive days, followed by Dose Level II (9×10⁹ cfu/dose) for 3 days, and then Dose Level III (1.4×10⁹ cfu/dose) for 2 days.

FIG. 8A depicts hemoglobin (gms %), white blood count and platelet count for patient #3 plotted on the Y axis and expressed as a function of treatment days, plotted on the X axis.

FIG. 8B depicts data indicating that REXIN-G has no adverse effects on for patient #3 liver function. AST (U/L) ALT (U/L), and bilirubin (mg %), plotted on the Y axis, are expressed as a function of treatment days, plotted on the X axis.

FIG. 8C depicts data indicating that REXIN-G has no adverse effects on for patient #3 kidney function. Blood urea nitrogen (mg %), creatinine (mg %) and potassium (mmol/L) levels, plotted on the Y axis, are expressed as a function of treatment days, plotted on the X axis. Dose Level I (4.5×10⁹ cfu/dose) was given for 6 consecutive days.

FIG. 9 depicts size measurements of REXIN-G nanoparticles. Using a Precision Detector Instrument (Franklin, Mass. 02038 U.S.A.), the vector samples were analyzed using Dynamic Light Scattering (DLS) in Batch Mode for determining molecular size as the hydrodynamic radius (rh). Precision Deconvolve software was used to mathematically determine the various size populations from the DLS data. The average particle size of 3 REXIN-G clinical lots are 95, 105 and 95 nm respectively with no detectable viral aggregation.

FIG. 10 depicts the High Infectious Titer (HIT) version of the GTI expression vector GlnXSvNa. The pRV109 plasmid provides the strong CMV promoter. The resulting pREX expression vector has an SV40 ori for episomal replication and plasmid rescue in producer cell lines expressing the SV40 large T antigen (293T), an ampicillin resistance gene for selection and maintenance in E. coli, and a neomycin resistance gene driven by the SV40 e.p. to determine vector titer. The gene of interest is initially cloned as a PCR product with Not I and Sal I overhangs. The amplified fragments are verified by DNA sequence analysis and inserted into the retroviral expression vector pREX by cloning the respective fragment into pG1XsvNa (Gene Therapy Inc.), then excising the Kpn I fragment of this plasmid followed by ligation with a linearized (Kpn I-digested) pRV109 plasmid to yield the respective HIT/pREX vector.

FIG. 11 depicts a map of pC-REX II (i.e., EPEIUS-REX) plasmid.

FIG. 12 depicts a map of the novel pC-REX II (i.e., EPEIUS-REX) plasmid with the therapeutic cytokine gene IL-2 inserted.

FIG. 13 depicts a map of the novel pC-REX II (i.e., EPEIUS-REX) plasmid with the therapeutic cytokine gene GM-CSF inserted.

FIG. 14A depicts a map of the novel pB-RVE plasmid, an enhanced CMV expression plasmid bearing a targeted retroviral vector envelope construct (Epeius-BV1): a minimal amphotropic env (4070A) modified by the addition of a unique restriction site near the N-terminus of the mature protein (CAE-P); engineered to exhibit a collagen-binding motif (GHVGWREPSFMALSAA) (SEQ ID NO:1); and re-generated by PCR to eliminate all upstream (5′) and downstream (3′) viral sequences. The plasmid backbone (phCMV1) provides an optimized CMV prompter/enhancer/intron to drive the expression of env, in addition to an SV40 promoter/enhancer, which enables episomal replication in vector producer cells expressing the SV40 large T antigen (293T). Positive selection is provided by the kanamycin resistance gene.

FIG. 14B depicts a restriction digest of pB-RVE.

FIG. 15A depicts a map of the novel pdnG1/UBER-REX plasmid. This plasmid encodes the 209 aa (630 bp) dominant-negative mutant dnG1 (472-1098 nt; 41-249 aa; Accession # U47413). The plasmid is derived from G1XSvNa (GTI), into which the CMV i.e. promoter enhancer was cloned at the unique Sac II site upstream of the 5′ LTR. 487 bp of residual gag sequences were removed (D) to reduce the possibility of RCR, and a 97 bp splice acceptor site (ESA) was added upstream of dnG1. The dnG1 coding sequence (nt 472-1098 plus stop codon=1101) was prepared by PCR, including Not I and Sal I overhangs. The neo gene is driven by the SV40 e.p. with its nested ori. The pdnG1/UBER-REX plasmid was designed for high-titer vector production in 293T cells

FIG. 15B depicts the restriction digest of pdnG1/UBER-REX.

FIG. 16A illustrates a schematic representation of the C-REX plasmid.

FIG. 16B illustrates a schematic representation of the UBER-REX plasmid.

FIG. 17 depicts intravenous REXIN-G induced necrosis and fibrosis in metastatic tumor nodules, as observed in surgically excised liver sections from a patient with Stage IV pancreatic cancer (Patient A3). (A) Representative hematoxylin-eosin stained tissue section of a tumor nodule in biopsied liver; t=tumor cells; n=necrosis; f=fibrosis. (B) Trichrome stain of a tissue section of same tumor nodule. Blue-staining material indicates presence of collagenous proteins in fibrotic areas.

FIG. 18 depicts intravenous REXIN-G induced overt apoptosis in metastatic tumor nodules, seen of a patient with pancreatic cancer (Patient A3). (A-D) Representative immunostained tissue sections of tumor nodules from biopsied liver indicating an appreciable incidence of Tunel-positive apoptotic nuclei (brown-staining material).

FIG. 19 depicts immunohistochemical characterization of tumor infiltrating lymphocytes (TILs) in metastatic tumor nodules excised from a REXIN-G-treated patient with pancreatic cancer (Patient A3). Representative tissue sections of residual tumor nodules within the biopsied liver show significant TIL infiltration with a functional complement of immunoreactive T and B cells. Clockwise from upper left: Helper T cells (cd4+), Killer T cells (cd8+), B cells (cd20+), Monocyte/Macrophages (cd45+), Dendritic cells (cd35+), and Natural Killer cells (cd56+). Note, the presence (i.e., migration) of a cadre of TILs that function in the context of cell-mediated and humoral immunity, suggests the potential for cancer immunization in an immune competent host.

FIG. 20 depicts intravenous REXIN-G induced necrosis, apoptosis and fibrosis in a cancerous lymph node of a patient with malignant melanoma (Patient B4). A) H&E stained tissue sections of inguinal lymph node revealing extensive necrosis (n), apoptosis (indicated by arrows) and fibrosis (f) of cancer cells with a rim of viable tumor cells in the periphery (t); (B) Higher magnification (100×) of sections of A showing numerous cells undergoing apoptosis indicated by small cells with pyknotic or fragmented nuclei; (C) Higher magnification (100×) of A revealing golden-yellow hemosiderin-laden macrophages; (D) Representative tissue sections of inguinal lymph node showing significant infiltration with immunoreactive CD35+ dendritic cells, (E) CD68+ macrophages and (F) CD8+ killer T cells.

FIG. 21 depicts evidence of tumor regression in a patient with squamous cell carcinoma of the larynx (Patient B6). MRI images of the neck region obtained before (upper panel) and after (lower panel) REXIN-G treatment. Measurement of the diameters of serial sections of the upper airway shows a dramatic (˜300%) increase in the upper airway diameters after repeated infusions of REXIN-G when compared to sections obtained prior to treatment (indicated by white arrows). The increased patency of the airway corresponded to regression of the surrounding tumor mass, and a return of vocal capabilities.

FIG. 22 depicts the effects of REXIN-G infusions on the number and quality of hepatic metastatic lesions observed in a pancreatic cancer patient exhibiting a massive tumor burden (Patient C1). Abdominal MRI obtained (A) before treatment and (B) after treatment with calculated (Calculus of Parity) dose-dense infusions of REXIN-G. Note the complete eradication of numerous small dense tumor nodules in the upper left quadrant of the image (bracketed), as well as cystic conversion of established liver nodules (black arrows). Subsequent aspiration of the enlarged liver cyst (white arrow) followed by cytological analysis confirmed the complete absence of cancer cells in the aspirates following the treatment.

FIG. 23 depicts the effects of treatment with REXIN-G on intractable osteosarcoma, metastatic to heart, lungs, and adrenal gland. Radiologic imaging identifies the major metastatic sites (A), focusing on three pulmonary target lesions (arrows) which change dramatically from baseline (B), to one month (C) to three months (D) of REXIN-G treatment. Notably, the densities of these tumors change significantly, indicating reactive calcification and necrosis, while the PET scan adds mechanistic details, confirming the cessation of tumor metabolic activity.

FIG. 24 depicts the effects of treatment with REXIN-G on intractable metastatic osteosarcoma wherein halting progression and stabilization of disease by REXIN-G, acting here as neoadjuvant and adjuvant therapy, enabled a surgical remission gained by the excision of two residual tumor nodules. Histological examination of the excised tumors demonstrated clear objective responses, confirming calcification (A, and C at higher magnification) in one lesion, and cystic conversion and necrosis (B, and D at higher magnification) of the second lesion following REXIN-G treatment.

FIG. 25 depicts the effects of treatment with REXIN-G on intractable Ewing's sarcoma, metastatic to the lungs and spine. A comparison of the PET scans with the CT scans of three large target lesions in the chest region (A) reveals a problematic disparity in evaluating objective clinical responses in tumor size versus tumor metabolism following REXIN-G treatment. Likewise, the diffuse metastatic tumor infiltration in the lumbar region (B), which was detected by PET scan but not CT scan, further suggests that clinical understanding based on tumor size alone is of a very meager kind.

FIG. 26 depicts the effects of treatment with REXIN-G on intractable metastatic breast cancer, revealing histological aspects of tumor destruction, reparative fibrosis, and reactive immune cell infiltration, now-classical hallmarks of REXIN-G action. In this excised tumor nodule, a scant number of tumor cells (tu) can be seen in the context of extensive fibrosis (fib) accompanied by a significant immune response (im) following REXIN-G treatment (A, H&E stain; B, Trichrome stain for extracellular matrix proteins). The remaining nests of degenerative tumor cells (marked in F) appear to be infiltrated and ‘recognized’ by the patient's immune cells (C, H&E; D, LCA immunostaining), including killer T-cells (E).

FIG. 27 depicts the effects of treatment with REXIN-G on intractable metastatic pancreatic cancer, wherein the patient received REXIN-G as second-line therapy treatment shortly after failing standard first line therapy; thus demonstrating the clinical benefit of gaining effective tumor control at a relatively early stage of disease progression. Complete regression of the primary pancreatic tumor (A versus B) is demonstrated along with both size (RECIST) and density (CHOI) changes in a metastatic liver lesion (C versus D); resulting in the stabilization of disease, prevention of new lesions, and enhancement of treatment options.

FIG. 28 depicts the effects of treatment with REXIN-G on recurrent chemotherapy-resistant pancreas cancer with metastasis to the liver and abdominal lymph nodes, documenting a complete clinical remission gained by continued treatment with REXIN-G as stand alone therapy. Graphic analysis of radiological images of tumor burden in the liver (A, Y-axis)) obtained during course of REXIN-G treatment (X-axis) demonstrated a halting of progression with stable disease (SD) and no new lesions; however, a slight rise in the size a liver lesion (determined solely by RECIST criteria) ‘appeared’ to indicate progressive disease (PD). A more comprehensive analysis of the eradication of tumor burden in the lymph nodes (B), including the levels of the CA19.9 tumor marker (C) which had dropped toward baseline, encouraged the oncologist to hold-the-course of the targeted therapy, thereby maintaining the conditions that led to a complete tumor response (CR) within the following month. The importance of holding the course of REXIN-G treatment, in the absence of systemic toxicity, in the absence of any new lesions and/or verifiable disease progression, is evident by the resulting sustained clinical remission.

FIG. 29 depicts the effects of treatment with REXIN-G on intractable metastatic pancreas cancer, wherein the surgical excision of a residual tumor from the liver provides important insights into the molecular mechanisms-of-action of REXIN-G, as well as a sustained clinical remission. Histological examination of the excised liver nodule (A) demonstrates the limitations of simple RECIST measurements, revealing epithelioid tumor cells (tu) in various stages of degeneration (insert) that are surrounded by a significant amount of reparative fibrosis (B, ECM stains blue) and immune cell infiltration (C, Leukocytes), including both helper T-cells (F) and killer T-cells (G). Most noteworthy is the direct anti-tumor action of REXIN-G, which is evidenced by the large amounts of apoptosis (active cell death) seen in the columnar/ductal arrays of tumor cells (D, TUNEL stain; E, Control); for the curative surgical excision of this nodule followed REXIN-G treatment, as neoadjuvant therapy.

FIG. 30 depicts a Kaplan Meier analysis of progression-free survival in REXIN-G-treated patients with bone and soft tissue sarcoma (A and B) and overall survival data of evaluable patients (C).

FIG. 31A depicts the overall survival data on evaluable osteosarcoma patients. Kaplan-Meier analysis shows Overall Survival curve of 17 evaluable patients with recurrent or metastatic osteosarcoma refractory to known therapies who completed at least one treatment cycle and had a tumor response evaluation.

FIG. 31B depicts the progression-free survival rates of patients with pancreatic cancer. The Kaplan-Meier plot for survival of 20 patients in the “Intention-to-Treat” patient population. The results indicate a dose-response relationship between overall survival and REXIN-G dosage (p=0.03).

FIG. 32 depicts a flow diagram of therapeutic embodiment using targeted vector therapy in combination with radiation or chemotherapeutic therapy.

### DETAILED DESCRIPTION OF THE INVENTION

The therapeutic systems disclosed herein targets retroviral vectors or any other viral or non-viral vector, protein or drug selectively to areas of pathology (i.e., pathotropic targeting), enabling preferential gene delivery to vascular (Hall et al., Hum Gene Ther, 8:2183-92, 1997; Hall et al., Hum Gene Ther, 11:983-93, 2000) or cancerous lesions (Gordon et al., Hum Gene Ther 12:193-204, 2001; Gordon et al., Curiel D T, Douglas J T, eds. Vector Targeting Strategies for Therapeutic Gene Delivery, New York, N.Y.: Wiley-Liss, Inc. 293-320, 2002), areas of active angiogenesis, and areas of tissue injury or inflammation with high efficiency in vivo. See also US Patent Publication Nos. 2004-0253215, 2007-0178066, 2009-0123428 and 2010-0016413, each of which are incorporated by reference in its entirety.

### DEFINITIONS

Unless defined otherwise, all technical and scientific terms used herein have the same meaning as is commonly understood by one of skill in the art to which the invention(s) belong. All patents, patent applications, published applications and publications, Genbank sequences, websites and other published materials referred to throughout the entire disclosure herein, unless noted otherwise, are incorporated by reference in their entirety. In the event that there are a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

As used herein, “nucleic acid” refers to a polynucleotide containing at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known (Lin et al. (1994) Nucl. Acids Res. 22:5220-5234; Jellinek et al. (1995) Biochemistry 34:11363-11372; Pagratis et al. (1997) Nature Biotechnol. 15:68-73). The nucleic acid can be single-stranded, double-stranded, or a mixture thereof. For purposes herein, unless specified otherwise, the nucleic acid is double-stranded, or it is apparent from the context.

As used herein, DNA is meant to include all types and sizes of DNA molecules including cDNA, plasmids and DNA including modified nucleotides and nucleotide analogs.

As used herein, nucleotides include nucleoside mono-, di-, and triphosphates. Nucleotides also include modified nucleotides, such as, but are not limited to, phosphorothioate nucleotides and deazapurine nucleotides and other nucleotide analogs.

As used herein, the term “subject” refers to animals, plants, insects, and birds into which the large DNA molecules can be introduced. Included are higher organisms, such as mammals and birds, including humans, primates, rodents, cattle, pigs, rabbits, goats, sheep, mice, rats, guinea pigs, cats, dogs, horses, chicken and others.

As used herein, “administering to a subject” is a procedure by which one or more delivery agents and/or large nucleic acid molecules, together or separately, are introduced into or applied onto a subject such that target cells which are present in the subject are eventually contacted with the agent and/or the large nucleic acid molecules.

As used herein, “targeted delivery vector” or “targeted delivery vehicle” or “targeted therapeutic vector” or “targeted therapeutic system” refers to both viral and non-viral particles that harbor and transport exogenous nucleic acid molecules to a target cell or tissue. Viral vehicles include, but are not limited to, retroviruses, adenoviruses and adeno-associated viruses. Non-viral vehicles include, but are not limited to, microparticles, nanoparticles, virosomes and liposomes. “Targeted,” as used herein, refers to the use of ligands that are associated with the delivery vehicle and target the vehicle to a cell or tissue. Ligands include, but are not limited to, antibodies, receptors and collagen binding domains.

As used herein, “delivery,” which is used interchangeably with “transduction,” refers to the process by which exogenous nucleic acid molecules are transferred into a cell such that they are located inside the cell. Delivery of nucleic acids is a distinct process from expression of nucleic acids.

As used herein, a “multiple cloning site (MCS)” is a nucleic acid region in a plasmid that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector.

As used herein, “origin of replication” (often termed “ori”), is a specific nucleic acid sequence at which replication is initiated. Alternatively an autonomously replicating sequence (ARS) can be employed if the host cell is yeast.

As used herein, “selectable or screenable markers” confer an identifiable change to a cell permitting easy identification of cells containing an expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al., Virology 52:456 (1973); Sambrook et al., Molecular Cloning: A Laboratory Manual (1989); Davis et al., Basic Methods in Molecular Biology (1986); Chu et al., Gene 13:197 (1981). Such techniques can be used to introduce one or more exogenous DNA moieties, such as a nucleotide integration vector and other nucleic acid molecules, into suitable host cells. The term captures chemical, electrical, and viral-mediated transfection procedures.

As used herein, “expression” refers to the process by which nucleic acid is translated into peptides or is transcribed into RNA, which, for example, can be translated into peptides, polypeptides or proteins. If the nucleic acid is derived from genomic DNA, expression may, if an appropriate eukaryotic host cell or organism is selected, include splicing of the mRNA. For heterologous nucleic acid to be expressed in a host cell, it must initially be delivered into the cell and then, once in the cell, ultimately reside in the nucleus.

As used herein, “applying to a subject” is a procedure by which target cells present in the subject are eventually contacted with energy such as ultrasound or electrical energy. Application is by any process by which energy can be applied.

As used herein, a “therapeutic course” refers to the periodic or timed administration of the targeted vectors disclosed herein within a defined period of time. Such a period of time is at least one day, at least two days, at least three days, at least five days, at least one week, at least two weeks, at least three weeks, at least one month, at least two months, or at least six months. Administration could also take place in a chronic manner, i.e. for an undefined period of time. The periodic or timed administration includes once a day, twice a day, three times a day or other set timed administration.

As used herein, the terms “co-administration,” “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments, a therapeutic agent as disclosed in the present application will be co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, a therapeutic agent and the other agent(s) are administered in a single composition. In some embodiments, a therapeutic agent and the other agent(s) are admixed in the composition. In further embodiments, a therapeutic agent and the other agent(s) are administered at separate times in separate doses.

The term “host cell” denotes, for example, microorganisms, yeast cells, insect cells, and mammalian cells, that can be, or have been, used as recipients for multiple constructs for producing a targeted delivery vector. The term includes the progeny of the original cell which has been transfected. Thus, a “host cell” as used herein generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, “genetic therapy” involves the transfer of heterologous DNA to the certain cells, target cells, of a mammal, particularly a human, with a disorder or conditions for which therapy or diagnosis is sought. The DNA is introduced into the selected target cells in a manner such that the heterologous DNA is expressed and a therapeutic product encoded thereby is produced. Alternatively, the heterologous DNA may in some manner mediate expression of DNA that encodes the therapeutic product, it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. The heterologous DNA encoding the therapeutic product may be modified prior to introduction into the cells of the afflicted host in order to enhance or otherwise alter the product or expression thereof.

As used herein, “heterologous nucleic acid sequence” is typically DNA that encodes RNA and proteins that are not normally produced in vivo by the cell in which it is expressed or that mediates or encodes mediators that alter expression of endogenous DNA by affecting transcription, translation, or other regulatable biochemical processes. A heterologous nucleic acid sequence may also be referred to as foreign DNA. Any DNA that one of skill in the art would recognize or consider as heterologous or foreign to the cell in which it is expressed is herein encompassed by heterologous DNA. Examples of heterologous DNA include, but are not limited to, DNA that encodes traceable marker proteins, such as a protein that confers drug resistance, DNA that encodes therapeutically effective substances, such as anti-cancer agents, enzymes and hormones, and DNA that encodes other types of proteins, such as antibodies. Antibodies that are encoded by heterologous DNA may be secreted or expressed on the surface of the cell in which the heterologous DNA has been introduced.

### Plasmids

Plasmids disclosed herein are used to transfect and produce targeted delivery vectors or targeted therapeutic vectors for use in therapeutic and diagnostic procedures. In general, such plasmids provide nucleic acid sequences that encode components, viral or non-viral, of targeted vectors disclosed herein. Such plasmids include nucleic acid sequences that encode, for example the 4070A amphotropic envelope protein modified to contain a collagen binding domain. Additional plasmids can include a nucleic acid sequence operably linked to a promoter. The sequence generally encodes a viral gag-pol polypeptide. The plasmid further includes a nucleic acid sequence operably linked to a promoter, and the sequence encodes a polypeptide that confers drug resistance on the producer cell. An origin of replication is also included. Additional plasmids can include a heterologous nucleic acid sequence encoding a diagnostic or therapeutic polypeptide, 5′ and 3′ long terminal repeat sequences; a Ψ retroviral packaging sequence, a CMV promoter upstream of the 5′ LTR, a nucleic acid sequence operably linked to a promoter, and an SV40 origin of replication.

The heterologous nucleic acid sequence generally encodes a diagnostic or therapeutic polypeptide. In specific embodiments, the therapeutic polypeptide or protein is a “suicide protein” that causes cell death by itself or in the presence of other compounds. A representative example of such a suicide protein is thymidine kinase of the herpes simplex virus. Additional examples include thymidine kinase of varicella zoster virus, the bacterial gene cytosine deaminase (which converts 5-fluorocytosine to the highly toxic compound 5-fluorouracil), p450 oxidoreductase, carboxypeptidase G2, beta-glucuronidase, penicillin-V-amidase, penicillin-G-amidase, beta-lactamase, nitroreductase, carboxypeptidase A, linamarase (also referred to as .beta.-glucosidase), the E. coli gpt gene, and the E. coli Deo gene, although others are known in the art. In some embodiments, the suicide protein converts a prodrug into a toxic compound. As used herein, “prodrug” means any compound useful in the methods of the present invention that can be converted to a toxic product, i.e. toxic to tumor cells. The prodrug is converted to a toxic product by the suicide protein. Representative examples of such prodrugs include: ganciclovir, acyclovir, and FIAU (1-(2-deoxy-2-fluoro-.beta.-D-arabinofuranosyl)-5-iod-ouracil) for thymidine kinase; ifosfamide for oxidoreductase; 6-methoxypurine arabinoside for VZV-TK; 5-fluorocytosine for cytosine deaminase; doxorubicin for beta-glucuronidase; CB1954 and nitrofurazone for nitroreductase; and N-(Cyanoacetyl)-L-phenylalanine or N-(3-chloropropionyl)-L-phenylalanine for carboxypeptidase A. The prodrug may be administered readily by a person having ordinary skill in this art. A person with ordinary skill would readily be able to determine the most appropriate dose and route for the administration of the prodrug.

In some embodiments, a therapeutic protein or polypeptide, is a cancer suppressor, for example p53 or Rb, or a nucleic acid encoding such a protein or polypeptide. Of course, those of skill know of a wide variety of such cancer suppressors and how to obtain them and/or the nucleic acids encoding them.

Other examples of therapeutic proteins or polypeptides include pro-apoptotic therapeutic proteins and polypeptides, for example, p15, p16, or p21/WAF-1.

Cytokines, and nucleic acid encoding them may also be used as therapeutic proteins and polypeptides. Examples include: GM-CSF (granulocyte macrophage colony stimulating factor); TNF-alpha (Tumor necrosis factor alpha); Interferons including, but not limited to, IFN-alpha and IFN-gamma; and Interleukins including, but not limited to, Interleukin-1 (IL1), Interleukin-Beta (IL-beta), Interleukin-2 (IL2), Interleukin-4 (IL4), Interleukin-5 (IL5), Interleukin-6 (IL6), Interleukin-8 (IL8), Interleukin-10 (IL10), Interleukin-12 (IL12), Interleukin-13 (IL13), Interleukin-14 (IL14), Interleukin-15 (ILLS), Interleukin-16 (IL16), Interleukin-18 (IL18), Interleukin-23 (IL23), Interleukin-24 (IL24), although other embodiments are known in the art.

Additional examples of cytocidal genes include, but are not limited to, mutated cyclin G1 genes. By way of example, the cytocidal gene may be a dominant negative mutation of the cyclin G1 protein (e.g., WO/01/64870).

Previously, retroviral vector (RV) constructs were generally produced by the cloning and fusion of two separate retroviral (RV) plasmids: one containing the retroviral LTRs, packaging sequences, and the respective gene(s) of interest; and another retroviral vector containing a strong promoter (e.g., CMV) as well as a host of extraneous functional sequences. The pC-REX II (e-REX) vector disclosed herein refers to an improved plasmid containing an insertion of a unique set of cloning sites in the primary plasmid to facilitate directional cloning of the experimental gene(s). The strong promoter (ex, CMV) is employed in the plasmid backbone to increase the amount of RNA message generated within the recipient producer cells but is not itself packaged into the retroviral particle, as it lies outside of the gene-flanking retroviral LTR's.

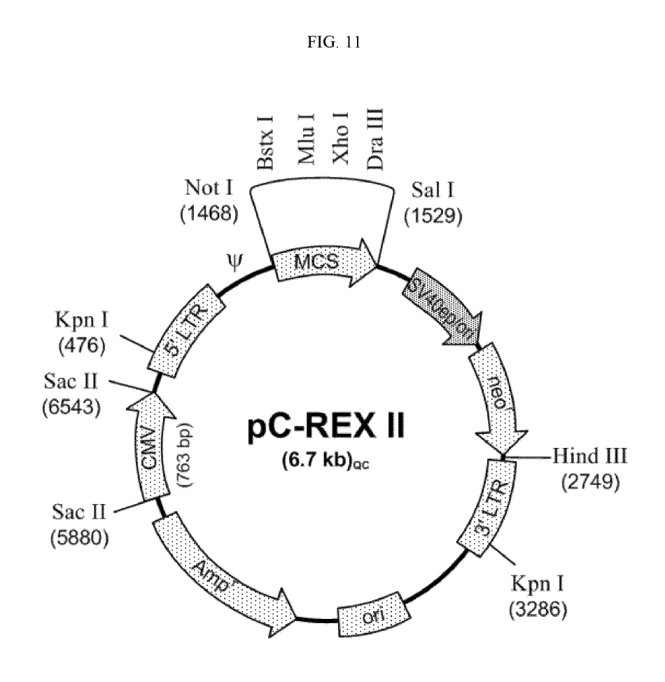

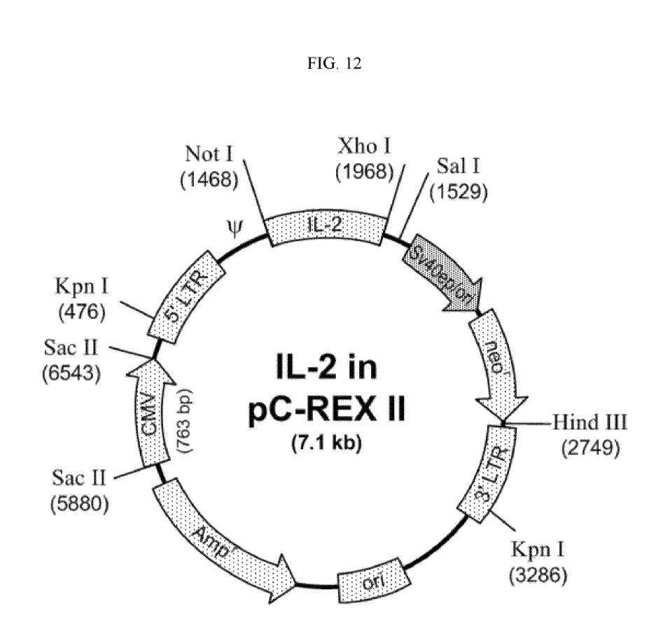

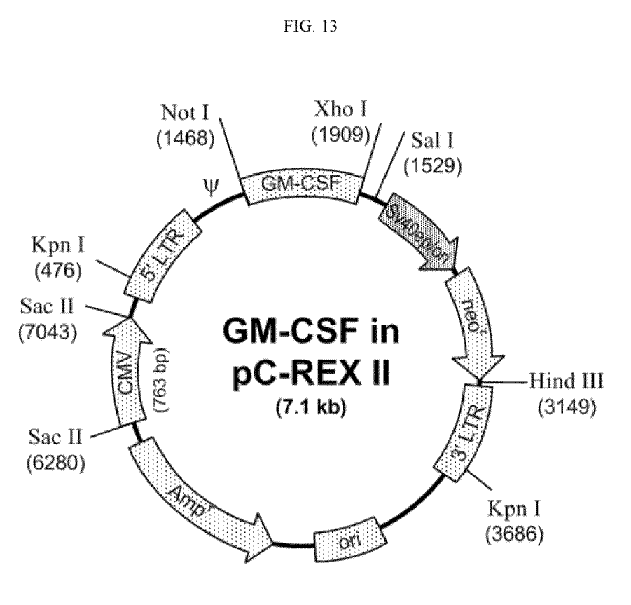

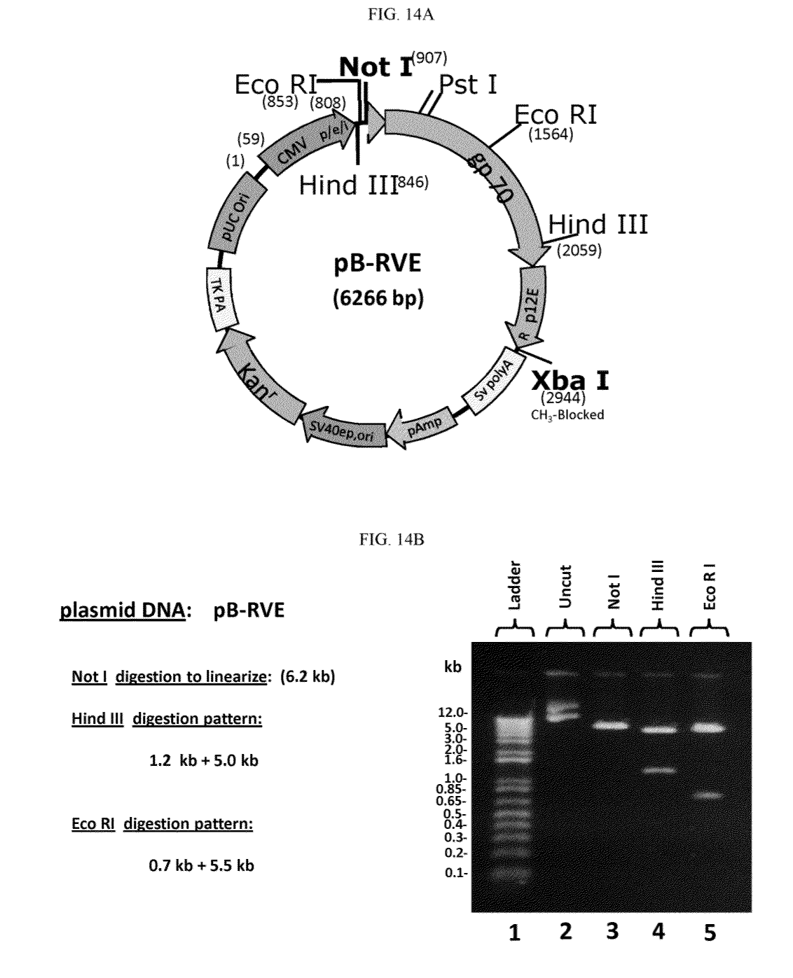

Therefore, an improved plasmid was designed which included the strong CMV promoter (obtained by PCR) into a strategic site within the G1xSvNa vector, which was previously approved for human use by the FDA, thus eliminating the plasmid size and sequence concerns of previously reported vectors. This streamlined construct was designated pC-REX. PC-REX was further modified to incorporate a series of unique cloning sites (see MCS in pC-REX II, FIG. 11), enabling directional cloning and/or the insertion of multiple genes as well as auxiliary functional domains. Thus, the new plasmids are designated pC-REX and pC-REX II (EPEIUS-REX or eREX). The pC-REX plasmid design outperformed that of pHIT-112/pREX in direct side-by-side comparisons. The new plasmid design was further modified to include the coding sequence of various therapeutically effective polypeptides. In one example, the dominant negative Cyclin G1 (dnG1) was included as the therapeutic gene. The tripartite viral particle (env, gag-pol, and dnG1 gene vector construct) has been referred to collectively as REXIN-G in published reports of the clinical trials. Thus, REXIN-G represents the targeted delivery vector dnG1/C-REX that is packaged, encapsidated, and enveloped in a targeted, injectable viral particle.

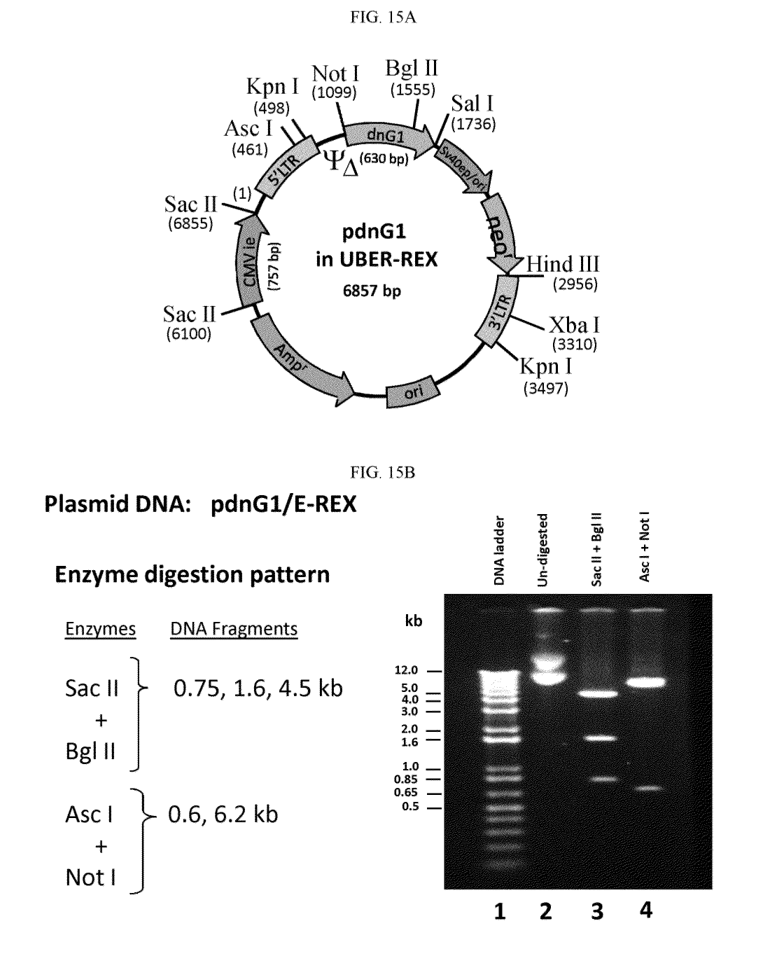

The incidence of replication-competent retrovirus in a transient plasmid co-transfection system such as the system used in REXIN-G production is unlikely, because the murine-based retroviral envelope construct, the packaging construct gag pol, and the retroviral vector are expressed in separate plasmids driven by their own promoters. Additionally, human producer cells are used to generate virions. Human cells do not have endogenous murine sequences that would be capable of recombining with a murine-based retroviral vector used in REXIN-G Recent improvements were made to the production of REXIN-G in order to further reduce the potential for generation of replication-competent retrovirus. The plasmid dnG1/C-REX contains residual gag-pol sequences that potentially overlap with 5′ DNA sequences contained in the respective gag-pol construct. Therefore, 487 base pairs were removed from the parent dnG1/C-REX plasmid followed by an insertion of 97 base pair splice acceptor site to yield pdnG1/UBER-REX (FIG. 15A).

A targeting ligand is included in a plasmid disclosed herein. Generally, it is inserted between two consecutively numbered amino acid residues of the native (i.e., unmodified) receptor binding region of the retroviral envelope encoded by a nucleic acid sequence of a plasmid, such as in the modified amphotropic CAE envelope polypeptide, wherein the targeting polypeptide is inserted between amino acid residues 6 and 7. The polypeptide is a portion of a protein known as gp70, which is included in the amphotropic envelope of Moloney Murine Leukemia Virus. In general, the targeting polypeptide includes a binding region which binds to an extracellular matrix component, including, but not limited to, collagen (including collagen Type I and collagen Type IV), laminin, fibronectin, elastin, glycosaminoglycans, proteoglycans, and sequences which bind to fibronectin, such as arginine-glycine-aspartic acid, or RGD, sequences. Binding regions which may be included in the targeting polypeptide include, but are not limited to, polypeptide domains which are functional domains within von Willebrand Factor or derivatives thereof, wherein such polypeptide domains bind to collagen. In one embodiment, the binding region is a polypeptide having the following structural formula: Trp-Arg-Glu-Pro-Ser-Phe-Met-Ala-Leu-Ser (SEQ ID NO: 3).

### Methods for Producing Targeted Vectors

This disclosure relates to the production of viral and non-viral vector particles, including retroviral vector particles, adenoviral vector particles, adeno-associated virus vector particles, Herpes Virus vector particles, pseudotyped viruses, and non-viral vectors having a modified, or targeted viral surface protein, such as, for example, a targeted viral envelope polypeptide, wherein such modified viral surface protein, such as a modified viral envelope polypeptide, includes a targeting polypeptide including a binding region which binds to an extracellular matrix component such as collagen. The targeting polypeptide may be placed between two consecutive amino acid residues of the viral surface protein, or may replace amino acid residues which have been removed from the viral surface protein.

One of the most frequently used delivery systems for achieving gene therapy involves viral vectors, most commonly adenoviral and retroviral vectors. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

For gene delivery purposes, a viral particle can be developed from a virus that is native to a target cell or from a virus that is non-native to a target cell. In general, it is desirable to use a non-native virus vector rather than a native virus vector. While native virus vectors may possess a natural affinity for target cells, such viruses pose a greater hazard since they possess a greater potential for propagation in target cells. In this regard, animal virus vectors, wherein they are not naturally designed for propagation in human cells, can be useful for gene delivery to human cells. In order to obtain sufficient yields of such animal virus vectors for use in gene delivery, however, it is necessary to carry out production in a native animal packaging cell. Virus vectors produced in this way, however, normally lack any components either as part of the envelope or as part of the capsid that can provide tropism for human cells. For example, current practices for the production of non-human virus vectors, such as ecotropic mouse (murine) retroviruses like MMLV, are produced in a mouse packaging cell line. Another component required for human cell tropism must be provided.

In general, the propagation of a viral vector (without a helper virus) proceeds in a packaging cell in which a nucleic acid sequence for packaging components were stably integrated into the cellular genome and nucleic acid coding for viral nucleic acid is introduced in such a cell line. Packaging lines currently available yield producer clones of sufficient titer to transduce human cells for gene therapy applications and have led to the initiation of human clinical trials. However, there are two areas in which these lines are deficient.

First, design of the appropriate retroviral vectors for particular applications requires the construction and testing of several vector configurations. For example, Belmont et al., Molec. and Cell. Biol. 8(12):5116-5125 (1988), constructed stable producer lines from 16 retroviral vectors in order to identify the vector capable of producing both the highest titer producer and giving optimal expression. Some of the configurations examined included: (1) LTR driven expression vs. an internal promoter; (2) selection of an internal promoter derived from a viral or a cellular gene; and (3) whether a selectable marker was incorporated in the construct. A packaging system that would enable rapid, high-titer virus production without the need to generate stable producer lines would be highly advantageous in that it would save approximately two months required for the identification of high titer producer clones derived from several constructs.

Second, compared to NIH 3T3 cells, the infection efficiency of primary cultures of mammalian somatic cells with a high titer amphotropic retrovirus producer varies considerably. The transduction efficiency of mouse myoblasts (Dhawan et al., Science 254:1509-1512 (1991) or rat capillary endothelial cells (Yao et. al., Proc. Natl. Acad. Sci. USA 88:8101-8105 (1991)) was shown to be approximately equal to that of NIH 3T3 cells, whereas the transduction efficiency of canine hepatocytes (Armentano et. al., Proc. Natl. Acad. Sci. USA 87:6141-6145 (1990)) was only 25% of that found in NIH 3T3 cells. Primary human tumor-infiltrating lymphocytes (“TILs”), human CD4+ and CD8+ T cells isolated from peripheral blood lymphocytes, and primate long-term reconstituting hematopoietic stem cells, represent an extreme example of low transduction efficiency compared to NIH 3T3 cells. Purified human CD4+ and CD8+ T Cells have been reported on one occasion to be infected to levels of 6%-9% with supernatants from stable producer clones (Morecki et al., Cancer Immunol. Immunother. 32:342-352 (1991)). If the retrovirus vector contains the neoR gene, populations that are highly enriched for transduced cells can be obtained by selection in G418. However, selectable marker expression has been shown to have deleterious effects on long-term gene expression in vivo in hematopoietic stem cells (Apperly et. al. Blood 78:310-317 (1991)).

To overcome these limitations, methods and compositions for novel transient transfection packaging systems are provided. Improvements in the retroviral vector design enables the following: (1) the replacement of cumbersome plasmid cloning and fusion procedures which represent the prior art, (2) the provision of a single straightforward plasmid construct which avoids undue fusions and mutations in the parent constructs, which would compromise the reagent in terms of gaining regulatory (i.e. FDA) approval, (3) the elimination of redundant, inoperative, and/or undesirable sequences in the resultant retroviral vector (4) greater flexibility in the selection and directional cloning of therapeutic gene constructs into the retroviral vector, (5) facilitation of the molecular cloning of various auxiliary domains within the retroviral vector, (6) the introduction of strategic modifications which demonstrably increase the performance of the parent plasmid in the context of vector producer cells, and thus, increasing the resulting potency of the retroviral vector product (7) significant reduction in the over-all size of the retroviral vector construct to the extent that plasmid production is increased from a “low copy, low yield” reagent in biologic fermentations to one of intermediate yield. Taken together, these modifications retain the virtues (in terms of vector safety, gene incorporation and gene expression) of retroviral vectors currently in use, while providing significant improvements in the construction, validation, manufacture, and performance of prospective retroviral vectors for human gene therapy. This represents the second component of TDS includes a high performance retroviral expression vector, designated the C-REX vector.

Transient transfection has numerous advantages over the packaging cell method. In this regard, transient transfection avoids the longer time required to generate stable vector-producing cell lines and is used if the vector genome or retroviral packaging components are toxic to cells. If the vector genome encodes toxic genes or genes that interfere with the replication of the host cell, such as inhibitors of the cell cycle or genes that induce apoptosis, it may be difficult to generate stable vector-producing cell lines, but transient transfection can be used to produce the vector before the cells die. Also, cell lines have been developed using transient infection that produce vector titer levels that are comparable to the levels obtained from stable vector-producing cell lines (Pear et al 1993, PNAS 90:8392-8396).

A high efficiency manufacturing process for large scale production of retroviral vector stock bearing cytocidal gene constructs with high bulk titer and biologic activity is provided. The manufacturing process describes the use of transiently transfected 293T producer cells; an engineered method of producer cell scale up; and a transient transfection procedure that generates retroviral vectors that retains cytocidal gene expression with high fidelity.

In another embodiment, a fully validated 293T (human embryonic kidney cells transformed with SV40 large T) master cell bank for clinical retroviral vector production is provided. Although 293T cells have generated small amounts of moderate to high titer vector stocks for laboratory use, these producer cells have not been shown previously to be useful for large scale production of clinical vector stocks. In yet other embodiments, the manufacturing process incorporates a method of DNA degradation in the preparation of the therapeutic retroviral product, including during the collection of the retroviral particles, the subsequent processing of the retroviral particles, the final steps of vector harvest and collection, the concentration of the retroviral particles, prior to storage of the therapeutic retroviral particles and/or just prior to administration of the retroviral particles that does not result in any loss of vector potency. DNA degradation steps may include treatment with DNase I (e.g. Pulmozyme (Genentech), TURBO™ Dnase (Ambion), Plasmid-Safe (Epicentre Technologies)). In some embodiments, from 0.1-10 Units/ml; 0.5-5 Units/ml; 1-4 Units/ml or 1 Unit/ml of DNase I is added to remove intact oncogenes from the therapeutic retroviral vector preparation.

In another embodiment, a method for concentrating retroviral vector stocks for therapeutic use and consistent generation of clinical vector products approaching 1×10⁹ cfu/ml is provided. In some embodiments, the concentration of the clinical vector products is at least 1×10⁷ cfu/ml. In other embodiments, the concentration of the clinical vector is at least 1×10⁸ cfu/ml. In yet other embodiments, the concentration of the clinical vector is at least 1×10⁹ cfu/ml. The final formulation of the clinical product consists of a chemically defined serum-free solution for harvest, collection and storage of high titer clinical vector stocks.

In another embodiment, a method of collection of the clinical vector or therapeutic retroviral vector particles using a system for maintenance of sterility, sampling of quality control specimens and facilitation of final fill, is provided. One example is a closed-loop manifold assembly designed to meet the specifications required for collection of clinical product, i.e., maintenance of sterility during sampling, and is not available as a product for sale. The closed loop manifold assembly for harvest of viral particles disclosed herein comprises a flexboy bag and manifold system made of Stedim 71 film; a 3 layer coextruded film consisting of a fluid contact layer of Ethyl Vinyl Acetate (EVA), a gas barrier of Ethyl Vinyl Alcohol (EVOH) and an outer layer of EVA. The total film thickness is 300 mm. EVA is an inert non-PVC-based film, which does not require the addition of plasticizers, thereby keeping extractables to a minimum. Stedim has conducted extensive biocompatibility trials and has established a Drug Master File with the FDA for this product. The film and port tubes meet USP Class VI requirements. All bag customization takes place in Stedim's class 10,000-controlled manufacturing environment. The film, tubing and all components used are gamma compatible to 45 kGy. Gamma irradiation is performed at a minimum exposure of 25 kGy to a maximum of 45 kGy. Product certificates of conformance are provided from both Stedim and their contract sterilizers. The closed-loop manifold system may also be used for the concentration, final fill and/or storage of the therapeutic retroviral vector particles. In yet other embodiments, the retroviral particles are collected and filter-sterilized using, for example, Amicon Ultrafree-MC centrifugal filters with 0.22 μm pore diameter (Millipore), or any other filter-sterilization system available. In still other embodiments, the retroviral vector particles are concentrated using centrifugation, flocculation, reagent binding, column purification and other means used to concentrate retroviral vector particles for clinical use.

The clinical retroviral vector may be stored at low temperatures, e.g. −80° C., for an extended period of time. The clinical retroviral vector may also be stored in volumes of 1 ml, 5 ml, 10 ml, 20 ml, 30 ml, 40 ml, 50 ml, 60 ml, 70 ml, 80 ml, 90 ml, 100 ml, 110 ml, 120 ml, 130 ml, 140 ml or 150 ml at −80° C. The clinical retroviral vector product may be stored in any suitable container that protects the product during long term, low-temperature storage conditions, including glass vials, cryobags and the like.

The fully validated product exhibits a viral titer of at least 1×10⁷ cfu/ml, at least 3×10⁷ cfu/ml, at least 5×10⁷ cfu/ml, at least 8×10⁷ cfu/ml, at least 1×10⁸ cfu/ml, at least 5×10⁸ cfu/ml, at least 1×10⁹ cfu/ml, at least 5×10⁹ cfu/ml, at least 1×10¹⁰ cfu/ml, or at least 5×10¹⁰ cfu/ml. The fully validated product may also have a biologic potency of at least 65-70%, at least 50-75%, at least 45-70%, at least 35-50%, at least 30%, at least 25%, at least 20% or at least 10% growth inhibitory activity in human breast, colon and pancreatic cancer cells. The fully validated product may also have a uniform particle size of ˜10 nm, ˜20 nm, ˜50 nm, ˜100 nm, ˜200 nm, ˜300 nm, ˜400 nm, ˜500 nm, ˜600 nm, ˜700 nm, ˜800 nm or ˜1000 nm with no viral aggregation. The fully validated product may also have less than 550 bp residual DNA, less than 500 bp residual DNA, less than 400 bp residual DNA, less than 300 bp residual DNA, less than 200 bp residual DNA or less than 100 bp residual DNA indicating absence of intact oncogenes. The fully validated may also have no detectable E1A or SV40 large T antigen, and no detectable replication competent retrovirus (RCR) in 5 passages on mus Dunni and human 293 cells. The fully validated product is sterile with an endotoxin level of <0.3 EU/ml, <0.2 EU/ml, <0.1 EU/ml, and the end of production cells are free of mycoplasma and other adventitious viruses.

REXIN-G produced using the new pB-RVE and pdnG1/UBER-REX plasmids was stored in volumes of 20-40 ml in 150 ml plastic cryobag at −70±10° C. The titers of the clinical lots ranged from 0.5 to 5.0×10e9 Units (U)/ml, and each lot was validated to be free of replication competent retrovirus (RCR), and of requisite purity, biological potency, sterility, and general safety for systemic use in humans.

The viral envelope includes a targeting ligand which includes, but are not limited to, the arginine-glycine-aspartic acid, or RGD, sequence, which binds fibronectin, and a polypeptide having the sequence Gly-Gly-Trp-Ser-His-Trp (SEQ ID NO:4), which also binds to fibronectin. In addition to the binding region, the targeting polypeptide may further include linker sequences of one or more amino acid residues, placed at the N-terminal and/or C-terminal of the binding region, whereby such linkers increase rotational flexibility and/or minimize steric hindrance of the modified envelope polypeptide. The polynucleotides may be constructed by genetic engineering techniques known to those skilled in the art.

Thus, a targeted delivery vector made in accordance with this invention contains associated therewith a ligand that facilitates the vector accumulation at a target site, i.e. a target-specific ligand. The ligand is a chemical moiety, such as a molecule, a functional group, or fragment thereof, which is specifically reactive with the target of choice while being less reactive with other targets thus giving the targeted delivery vector an advantage of transferring nucleic acids encoding therapeutic or diagnostic polypeptides, selectively into the cells in proximity to the target of choice. By being “reactive” it is meant having binding affinity to a cell or tissue, or being capable of internalizing into a cell wherein binding affinity is detectable by any means known in the art, for example, by any standard in vitro assay such as ELISA, flow cytometry, immunocytochemistry, surface plasmon resonance, etc. Usually a ligand binds to a particular molecular moiety—an epitope, such as a molecule, a functional group, or a molecular complex associated with a cell or tissue, forming a binding pair of two members. It is recognized that in a binding pair, any member may be a ligand, while the other being an epitope. Such binding pairs are known in the art. Exemplary binding pairs are antibody-antigen, hormone-receptor, enzyme-substrate, nutrient (e.g. vitamin)-transport protein, growth factor-growth factor receptor, carbohydrate-lectin, and two polynucleotides having complementary sequences. Fragments of the ligands are to be considered a ligand and may be used for the present invention so long as the fragment retains the ability to bind to the appropriate cell surface epitope. Preferably, the ligands are proteins and peptides comprising antigen-binding sequences of an immunoglobulin. More preferably, the ligands are antigen-binding antibody fragments lacking Fc sequences. Such preferred ligands are Fab fragments of an immunoglobulin, F(ab)2 fragments of immunoglobulin, Fv antibody fragments, or single-chain Fv antibody fragments. These fragments can be enzymatically derived or produced recombinantly. In their functional aspect, the ligands are preferably internalizable ligands, i.e. the ligands that are internalized by the cell of choice for example, by the process of endocytosis. Likewise, ligands with substitutions or other alterations, but which retain the epitope binding ability, may be used. The ligands are advantageously selected to recognize pathological cells, for example, malignant cells or infectious agents. Ligands that bind to exposed collagen, for example, can target the vector to an area of a subject that comprises malignant tissue. In general, cells that have metastasized to another area of a body do so by invading and disrupting healthy tissue. This invasion results in exposed collagen which can be targeted by the vectors provided herein.

An additional group of ligands that can be used to target a vector are those that form a binding pair with the tyrosine kinase growth factor receptors which are overexpressed on the cell surfaces in many tumors. Exemplary tyrosine kinase growth factors are VEGF receptor, FGF receptor, PDGF receptor, IGF receptor, EGF receptor, TGF-alpha receptor, TGF-beta receptor, HB-EGF receptor, ErbB2 receptor, ErbB3 receptor, and ErbB4 receptor. EGF receptor vIII and ErbB2 (HEr2) receptors are especially preferred in the context of cancer treatment using INSERTS as these receptors are more specific to malignant cells, while scarce on normal ones. Alternatively, the ligands are selected to recognize the cells in need of genetic correction, or genetic alteration by introduction of a beneficial gene, such as: liver cells, epithelial cells, endocrine cells in genetically deficient organisms, in vitro embryonic cells, germ cells, stem cells, reproductive cells, hybrid cells, plant cells, or any cells used in an industrial process.

The ligand may be expressed on the surface of a viral particle or attached to a non-viral particle by any suitable method available in the art. The attachment may be covalent or non-covalent, such as by adsorption or complex formation. The attachment preferably involves a lipophilic molecular moiety capable of conjugating to the ligand by forming a covalent or non-covalent bond, and referred to as an “anchor”. An anchor has affinity to lipophilic environments such as lipid micelles, bilayers, and other condensed phases, and thereby attaches the ligand to a lipid-nucleic acid microparticle. Methods of the ligand attachment via a lipophilic anchor are known in the art. (see, for example, F. Schuber, “Chemistry of ligand-coupling to liposomes”, in: Liposomes as Tools for Basic Research and Industry, ed. by J. R. Philippot and F. Schuber, CRC Press, Boca Raton, 1995, p. 21-37).

It is recognized that the targeted delivery vectors or targeted therapeutic vectors disclosed herein include viral and non-viral particles. Non-viral particles include encapsulated nucleoproteins, including wholly or partially assembled viral particles, in lipid bilayers. Methods for encapsulating viruses into lipid bilayers are known in the art. They include passive entrapment into lipid bilayer-enclosed vesicles (liposomes), and incubation of virions with liposomes (U.S. Pat. No. 5,962,429; Fasbender, et al., J. Biol. Chem. 272:6479-6489; Hodgson and Solaiman, Nature Biotechnology 14:339-342 (1996)). Without being limited by a theory, we assume that acidic proteins exposed on the surface of a virion provide an interface for complexation with the cationic lipid/cationic polymer component of the targeted delivery vector or targeted therapeutic vector and serve as a “scaffold” for the bilayer formation by the neutral lipid component. Exemplary types of viruses are adenoviruses, retroviruses, herpesviruses, lentiviruses, and bacteriophages.

Non-viral delivery systems, such as microparticles or nanoparticles including, for example, cationic liposomes and polycations, provide alternative methods for delivery systems and are encompassed by the present disclosure.

Examples of non-viral delivery systems include, for example, Wheeler et al., U.S. Pat. Nos. 5,976,567 and 5,981,501. These patents disclose preparation of serum-stable plasmid-lipid particles by contacting an aqueous solution of a plasmid with an organic solution containing cationic and non-cationic lipids. Thierry et al., U.S. Pat. No. 6,096,335 disclose preparing of a complex comprising a globally anionic biologically active substance, a cationic constituent, and an anionic constituent. Allen and Stuart, PCT/US98/12937 (WO 98/58630) disclose forming polynucleotide-cationic lipid particles in a lipid solvent suitable for solubilization of the cationic lipid, adding neutral vesicle-forming lipid to the solvent containing the particles, and evaporating the lipid solvent to form liposomes having the polynucleotide entrapped within. Allen and Stuart, U.S. Pat. No. 6,120,798, disclose forming polynucleotide-lipid microparticles by dissolving a polynucleotide in a first, e.g. aqueous, solvent, dissolving a lipid in a second, e.g. organic, solvent immiscible with said first solvent, adding a third solvent to effect formation of a single phase, and further adding an amount of the first and second solvents to effect formation of two liquid phases. Bally et al. U.S. Pat. No. 5,705,385, and Zhang et al. U.S. Pat. No. 6,110,745 disclose a method for preparing a lipid-nucleic acid particle by contacting a nucleic acid with a solution containing a non-cationic lipid and a cationic lipid to form a lipid-nucleic acid mixture. Maurer et al., PCT/CA00/00843 (WO 01/06574) disclose a method for preparing fully lipid-encapsulated therapeutic agent particles of a charged therapeutic agent including combining preformed lipid vesicles, a charged therapeutic agent, and a destabilizing agent to form a mixture thereof in a destabilizing solvent that destabilizes, but does not disrupt, the vesicles, and subsequently removing the destabilizing agent.

A Particle-Forming Component (“PFC”) typically comprises a lipid, such as a cationic lipid, optionally in combination with a PFC other than a cationic lipid. A cationic lipid is a lipid whose molecule is capable of electrolytic dissociation producing net positive ionic charge in the range of pH from about 3 to about 10, preferably in the physiological pH range from about 4 to about 9. Such cationic lipids encompass, for example, cationic detergents such as cationic amphiphiles having a single hydrocarbon chain. Patent and scientific literature describes numerous cationic lipids having nucleic acid transfection-enhancing properties. These transfection-enhancing cationic lipids include, for example: 1,2-dioleyloxy-3-(N,N,N-trimethylammonio)propane chloride-, DOTMA (U.S. Pat. No. 4,897,355); DOSPA (see Hawley-Nelson, et al., Focus 15(3):73 (1993)); N,N-distearyl-N,N-dimethyl-ammonium bromide, or DDAB (U.S. Pat. No. 5,279,833); 1,2-dioleoyloxy-3-(N,N,N-trimethylammonio) propane chloride-DOTAP (Stamatatos, et al., Biochemistry 27: 3917-3925 (1988)); glycerol based lipids (see Leventis, et al., Biochem. Biophys. Acta 1023:124 (1990); arginyl-PE (U.S. Pat. No. 5,980,935); lysinyl-PE (Puyal, et al. J. Biochem. 228:697 (1995)), lipopolyamines (U.S. Pat. No. 5,171,678) and cholesterol based lipids (WO 93/05162, U.S. Pat. No. 5,283,185); CHIM (1-(3-cholesteryl)-oxycarbonyl-aminomethylimidazole); and the like. Cationic lipids for transfection are reviewed, for example, in: Behr, Bioconjugate Chemistry, 5:382-389 (1994). Preferable cationic lipids are DDAB, CHIM, or combinations thereof. Examples of cationic lipids that are cationic detergents include (C12-C18)-alkyl- and (C12-C18)-alkenyl-trimethylammonium salts, N—(C12-C18)-alkyl- and N—(C12-C18)-alkenyl-pyridinium salts, and the like.

The size of a targeted delivery vector or targeted therapeutic vector formed in accordance with this invention is within the range of about 40 to about 1500 nm, preferably in the range of about 50-500 nm, and most preferably, in the range of about 20-150 nm. This size selection advantageously aids the targeted delivery vector, when it is administered to the body, to penetrate from the blood vessels into the diseased tissues such as malignant tumors, and transfer a therapeutic nucleic acid therein. It is also a characteristic and advantageous property of the targeted delivery vector that its size, as measured for example, by dynamic light scattering method, does not substantially increase in the presence of extracellular biological fluids such as in vitro cell culture media or blood plasma.

Alternatively, as described in Culver et al (1992) Science 256, 1550-1552, cells which produce retroviruses can be injected into a tumor. The retrovirus-producing cells so introduced are engineered to actively produce a targeted delivery vector, such as a viral vector particle, so that continuous productions of the vector occurred within the tumor mass in situ. Thus, proliferating tumor cells can be successfully transduced in vivo if mixed with retroviral vector-producing cells.

### Methods of Treatment

The targeted vectors of the present invention can also be used as a part of a gene therapy protocol to deliver nucleic acids encoding a therapeutic agent, such a mutant cyclin-G polypeptide. Thus, another aspect of the invention features expression vectors for in vivo or in vitro transfection of a therapeutic agent to areas of a subject comprising cell types associated with metastasized neoplastic disorders. The targeted vectors provided herein are intended for use as vectors for gene therapy. The mutant cyclin-G polypeptide and nucleic acid molecules can be used to replace the corresponding gene in other targeted vectors. Alternatively, a targeted vector disclosed herein (e.g., one comprising a collagen binding domain) can contain nucleic acid encoding any therapeutically agent (e.g., thymidine kinase). Of interest are those therapeutic agents useful for treating neoplastic disorders.

The present studies provide data generated from in vivo human clinical trials. Nevertheless, additional toxicity and therapeutic efficacy of a targeted vectors disclosed herein can be determined by standard pharmaceutical procedures in cell cultures or experimental animals, e.g., for determining the LDS₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Doses that exhibit large therapeutic indices are preferred. In the present invention, doses that would normally exhibit toxic side effects may be used because the therapeutic system is designed to target the site of treatment in order to minimize damage to untreated cells and reduce side effects.

The data obtained from human clinical trials (see below) prove that the targeted vector of the invention functions in vivo to inhibit the progression of a neoplastic disorder. The data in Table 1 provides a treatment regimen for administration of such a vector to a patient. In addition, data obtained from cell culture assays and animal studies using alternative forms of the targeted vector (e.g., alternative targeting mechanism or alternative therapeutic agent) can be used in formulating a range of dosage for use in humans. The dosage lies preferably within a range of circulating concentrations that include the ED₅₀ with little or no toxicity. The dosage may vary within this range depending upon the dosage form employed and the route of administration utilized. A therapeutically effective dose can be estimated initially from cell culture assays. A dose may be formulated in animal models to achieve a circulating plasma concentration range that includes the IC₅₀ (i.e., the concentration of the test compound which achieves a half-maximal infection or a half-maximal inhibition) as determined in cell culture. Such information can be used to more accurately determine useful doses in humans. Levels in plasma may be measured, for example, by high performance liquid chromatography.

Pharmaceutical compositions containing a targeted delivery vector can be formulated in any conventional manner by mixing a selected amount of the vector with one or more physiologically acceptable carriers or excipients. For example, the targeted delivery vector may be suspended in a carrier such as PBS (phosphate buffered saline). The active compounds can be administered by any appropriate route, for example, orally, parenterally, intravenously, intradermally, subcutaneously, or topically, in liquid, semi-liquid or solid form and are formulated in a manner suitable for each route of administration.

The targeted delivery vector may also be administered to increase local concentration of the vectors. For example, the targeted delivery vector may be administered via intra-arterial infusion, which increases local concentration of the targeted delivery vector to a specific organ system. Dependent upon the location of the target lesions, catheterization of the hepatic artery followed by infusion into the pancreaticoduodenal, right hepatic, and middle hepatic artery, respectively, may take place that could locally target hepatic lesions. Localized distribution of the targeted delivery vector may be directed to other organ systems, including the lung, gastrointestinal, brain, reproductive, splenic or other defined organ system via catheterization or other localized delivery system. Intra-arterial infusions may also take place via any other available arterial source, including but not limited to infusion through the hepatic artery, cerebral artery, coronary artery, pulmonary artery, iliac artery, celiac trunk, gastric artery, splenic artery, renal artery, gonadal artery, subclavian artery, vertebral artery, axilary artery, brachial artery, radial artery, ulnar artery, carotid artery, femoral artery, inferior mesenteric artery and/or superior mesenteric artery. Intra-arterial infusion may be accomplished using endovascular procedures, percutaneous procedures or open surgical approaches.

The targeted delivery vector and physiologically acceptable salts and solvates may be formulated for administration by inhalation or insufflation (either through the mouth or the nose) or for oral, buccal, parenteral or rectal administration. For administration by inhalation, the targeted delivery vector can be delivered in the form of an aerosol spray presentation from pressurized packs or a nebulizer, with the use of a suitable propellant, e.g. dichlorodifluoromethane, trichlorofluoromethane, dichlorotetra-fluoroetha-ne, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a therapeutic compound and a suitable powder base such as lactose or starch.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner.

The targeted delivery vector may be formulated for parenteral administration by injection e.g. by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder lyophilized form for constitution with a suitable vehicle, e.g., sterile pyrogen-free water, before use.

In addition to the formulations described previously, the targeted delivery vector may also be formulated as a depot preparation. Such long acting formulations may be administered by implantation (for example, subcutaneously or intramuscularly) or by intramuscular injection. Thus, for example, the therapeutic compounds may be formulated with suitable polymeric or hydrophobic materials (for example as an emulsion in an acceptable oil) or ion exchange resins, or as sparingly soluble derivatives, for example, as a sparingly soluble salt.

The active agents may be formulated for local or topical application, such as for topical application to the skin and mucous membranes, such as in the eye, in the form of gels, creams, and lotions and for application to the eye or for intracisternal or intraspinal application. Such solutions, particularly those intended for ophthalmic use, may be formulated as 0.01%-10% isotonic solutions, pH about 5-7, with appropriate salts. The compounds may be formulated as aerosols for topical application, such as by inhalation.

The concentration of active compound in the drug composition will depend on absorption, inactivation and excretion rates of the active compound, the dosage schedule, and amount administered as well as other factors known to those of skill in the art. For example, the amount that is delivered is sufficient to treat the symptoms of hypertension.

The compositions may, if desired, be presented in a pack or dispenser device which may contain one or more unit dosage forms containing the active ingredient. The pack may for example, comprise metal or plastic foil, such as a blister pack. The pack or dispenser device may be accompanied by instructions for administration.

The active agents may be packaged as articles of manufacture containing packaging material, an agent provided herein, and a label that indicates the disorder for which the agent is provided.

The targeted retroviral particle comprising the cytokine gene may be administered alone or in conjunction with other therapeutic treatments or active agents. For example, the targeted retroviral particle comprising a cytokine gene may be administered with the targeted retroviral particle comprising a cytocidal gene. The quantity of the targeted retroviral particle comprising a cytocidal gene to be administered is based on the titer of the virus particles as described herein above. By way of example, if the targeted retroviral particle comprising a cytokine gene is administered in conjunction with a targeted retroviral particle comprising a cytocidal gene the titer of the retroviral particle for each vector may be lower than if each vector is used alone. The targeted retroviral particle comprising the cytokine gene may be administered concurrently or separately from the targeted retroviral particle comprising the cytocidal gene.

The methods of the subject invention also relate to methods of treating cancer by administering a targeted retroviral particle (e.g., the targeted retroviral vector expressing a cytokine either alone or in conjunction with the targeted retroviral vector expressing a cytocidal gene) with one or more other active agents. Examples of other active agents that may be used include, but are not limited to, chemotherapeutic agents, anti-inflammatory agents, protease inhibitors, such as HIV protease inhibitors, nucleoside analogs, such as AZT. The one or more active agents may be administered concurrently or separately (e.g., before administration of the targeted retroviral particle or after administration of the targeted retroviral particle) with the one or more active agents. One of skill in the art will appreciate that the targeted retroviral particle may be administered either by the same route as the one or more agents (e.g., the targeted retroviral vector and the agent are both administered intravenously) or by different routes (e.g., the targeted retroviral vector is administered intravenously and the one or more agents are administered orally).

An effective amount or therapeutically effective of the targeted retroviral particles to be administered to a subject in need of treatment may be determined in a variety of ways. By way of example, the amount may be based on viral titer or efficacy in an animal model. Alternatively the dosing regimes used in clinical trials may be used as general guidelines. The daily dose may be administered in a single dose or in portions at various hours of the day. Initially, a higher dosage may be required and may be reduced over time when the optimal initial response is obtained. By way of example, treatment may be continuous for days, weeks, or years, or may be at intervals with intervening rest periods. The dosage may be modified in accordance with other treatments the individual may be receiving. However, the method of treatment is in no way limited to a particular concentration or range of the targeted retroviral particle and may be varied for each individual being treated and for each derivative used.

One of skill in the art will appreciate that individualization of dosage may be required to achieve the maximum effect for a given individual. It is further understood by one skilled in the art that the dosage administered to an individual being treated may vary depending on the individuals age, severity or stage of the disease and response to the course of treatment. One skilled in the art will know the clinical parameters to evaluate to determine proper dosage for the individual being treated by the methods described herein. Clinical parameters that may be assessed for determining dosage include, but are not limited to, tumor size, alteration in the level of tumor markers used in clinical testing for particular malignancies. Based on such parameters the treating physician will determine the therapeutically effective amount to be used for a given individual. Such therapies may be administered as often as necessary and for the period of time judged necessary by the treating physician.

The targeted therapeutic vectors, including but not limited to the targeted therapeutic retroviral particles, may be systemically or regionally (locally) delivered to a subject in need of treatment. For example, the targeted therapeutic vectors may be systemically administered intravenously. Alternatively, the targeted therapeutic vectors may also be administered intra-arterially. The targeted therapeutic vectors may also be administered topically, intravenously, intra-arterially, intracolonically, intratracheally, intraperitoneally, intranasally, intravascularly, intrathecally, intracranially, intramarrowly, intrapleurally, intradermally, subcutaneously, intramuscularly, intraocularly, intraosseously and/or intrasynovially. A combination of delivery modes may also be used, for example, a patient may receive the targeted therapeutic vectors both systemically and regionally (locally) to improve tumor responses with treatment of the targeted therapeutic vectors.

In some embodiments, multiple therapeutic courses (e.g. first and second therapeutic course) may be administered to a subject in need of treatment. In some embodiments, the first and/or second therapeutic course is administered intravenously. In other embodiments, the first and/or second therapeutic course is administered via intra-arterial infusion, including but not limited to infusion through the hepatic artery, cerebral artery, coronary artery, pulmonary artery, iliac artery, celiac trunk, gastric artery, splenic artery, renal artery, gonadal artery, subclavian artery, vertebral artery, axilary artery, brachial artery, radial artery, ulnar artery, carotid artery, femoral artery, inferior mesenteric artery and/or superior mesenteric artery. Intra-arterial infusion may be accomplished using endovascular procedures, percutaneous procedures or open surgical approaches. In some embodiments, the first and second therapeutic course may be administered sequentially. In yet other embodiments, the first and second therapeutic course may be administered simultaneously. In still other embodiments, the optional third therapeutic course may be administered sequentially or simultaneously with the first and second therapeutic courses.

In some embodiments, the targeted delivery vectors disclosed herein may be administered in conjunction with a sequential or concurrently administered therapeutic course(s) in high doses on a cumulative basis. For example, in some embodiments, a patient in need thereof may be systemically administered, e.g. intravenously administered, with a first therapeutic course of at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis. The first therapeutic course may be systemically administered. Alternatively, the first therapeutic course may be administered in a localized manner, e.g. intra-arterially, for example a patient in need thereof may be administered via intra-arterial infusion with at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis.

In yet other embodiments, a patient in need thereof may receive a combination, either sequentially or concurrently, of systemic and intra-arterial infusions administration of high doses of targeted delivery vector. For example, a patient in need thereof may be first systemically administered with at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis, followed by an additional therapeutic course of intra-arterial infusion, e.g. hepatic arterial infusion, administered targeted delivery vector of at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu on a cumulative basis. In still another embodiment, a patient in need thereof may receive a combination of intra-arterial infusion and systemic administration of targeted delivery vector in high doses. For example, a patient in need thereof may be first be administered via intra-arterial infusion with at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis, followed by an additional therapeutic course of systemically administered targeted delivery vector of at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu on a cumulative basis. The therapeutic courses may also be administered simultaneously, i.e. a therapeutic course of high doses of targeted delivery vector, for example, at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis, together with a therapeutic course of intra-arterial infusion, e.g. hepatic arterial infusion, administered targeted delivery vector of at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu on a cumulative basis.

In still other embodiments, a patient in need thereof may additionally receive, either sequentially or concurrently with the first and second therapeutic courses, additional therapeutic courses (e.g. third therapeutic course, fourth therapeutic course, fifth therapeutic course) of cumulative dose of targeted delivery vector, for example, at least 1×10⁹ cfu, at least 1×10¹⁰ cfu, at least 1×10¹¹ cfu, at least 1×10¹² cfu, at least 1×10¹³ cfu, at least 1×10¹⁴ cfu or at least 1×10¹⁵ cfu targeted delivery vector on a cumulative basis.

In some embodiments, the patient in need of treatment may be administered systemically (e.g. intravenously) a cumulative dose of at least 1×10¹¹ cfu, followed by the administration via intra-arterial infusion (e.g. hepatic-arterial infusion) of a cumulative dose of at least 1×10¹¹ cfu. In other embodiments, the patient in need of treatment may be administered systemically (e.g. intravenously) a cumulative dose of at least 1×10¹² cfu, followed by the administration via intra-arterial infusion (e.g. hepatic-arterial infusion) of a cumulative dose of at least 1×10¹² cfu. In one embodiment, the patient in need of treatment may be administered systemically (e.g. intravenously) a cumulative dose of at least 1×10¹³ cfu, followed by the administration via intra-arterial infusion (e.g. hepatic-arterial infusion) of a cumulative dose of at least 1×10¹³ cfu. In still other embodiments, the patient in need of treatment may be administered systemically (e.g. intravenously) a cumulative dose of at least 1×10¹¹ cfu, concurrently with the administration via intra-arterial infusion (e.g. hepatic-arterial infusion) of a cumulative dose of at least 1×10¹¹ cfu. In yet other embodiments, the patient in need of treatment may be administered systemically (e.g. intravenously) a cumulative dose of at least 1×10¹² cfu, concurrently with the administration via intra-arterial infusion (e.g. hepatic-arterial infusion) of a cumulative dose of at least 1×10¹² cfu. In still other embodiments, the patient in need of treatment may be administered systemically (e.g. intravenously) a cumulative dose of at least 1×10¹³ cfu, together with the administration via intra-arterial infusion (e.g. hepatic-arterial infusion) of a cumulative dose of at least 1×10¹³ cfu.

A patient in need of treatment may also be administered, either systemically or localized (for example intra-arterial infusion, such as hepatic arterial infusion) a therapeutic course of targeted delivery vector for a defined period of time. In some embodiments, the period of time may be at least one day, at least two days, at least three days, at least four days, at least five days, at least six days, at least seven days, at least one week, at least two weeks, at least three weeks, at least four weeks, at least five weeks, at least six weeks, at least seven weeks, at least eight weeks, at least 2 months, at least three months, at least four months, at least five months, at least six months, at least seven months, at least eight months, at least nine months, at least ten months, at least eleven months, at least one year, at least two years, at least three years, at least four years, or at least five years. Administration could also take place in a chronic manner, i.e. for an undefined or indefinite period of time.

Administration of the targeted delivery vector may also occur in a periodic manner, e.g., at least once a day, at least twice a day, at least three times a day, at least four times a day, at least five times a day. Periodic administration of the targeted delivery vector may be dependent upon the time of targeted delivery vector as well as the mode of administration. For example, parenteral administration may take place only once a day over an extended period of time, whereas oral administration of the targeted delivery vector may take place more than once a day wherein administration of the targeted delivery vector takes place over a shorter period of time.

In one embodiment, the subject is allowed to rest 1 to 2 days between the first therapeutic course and second therapeutic course. In some embodiments, the subject is allowed to rest 2 to 4 days between the first therapeutic course and second therapeutic course. In other embodiments, the subject is allowed to rest at least 2 days between the first and second therapeutic course. In yet other embodiments, the subject is allowed to rest at least 4 days between the first and second therapeutic course. In still other embodiments, the subject is allowed to rest at least 6 days between the first and second therapeutic course. In some embodiments, the subject is allowed to rest at least 1 week between the first and second therapeutic course. In yet other embodiments, the subject is allowed to rest at least 2 weeks between the first and second therapeutic course. In one embodiment, the subject is allowed to rest at least one month between the first and second therapeutic course. In some embodiments, the subject is allowed to rest at least 1-7 days between the second therapeutic course and the optional third therapeutic course. In yet other embodiments, the subject is allowed to rest at least 1-2 weeks between the second therapeutic course and the optional third therapeutic course.

The use of the improved pB-RVE and pdnG1/UBER-REX plasmids has allowed the production of a very high-potency preparation (1-5×10e9 cfu/ml) of REXIN-G™. This overcomes the problems of large infusion volume and resultant dosing limitations of the previous product and allows the development of strategic dose-dense regimens defined as the Calculus of Parity. In cancer therapy, a critical factor influencing the efficacy of an investigational agent is the extent of the tumor burden. Oftentimes, the margin of safety of a test drug is too narrow because dose-limiting toxicity is reached prior to gaining tumor control. Thus, the development of a cancer drug that can actually address the tumor burden without eliciting dose-limiting side effects or organ damage represents a significant milestone and advancement in cancer treatment. Another important problem is the natural kinetics of cancer growth, which requires an appropriate kinetic solution. Historic models of tumor growth are now considered overly simplistic (Heitjan (1991) Stat. Med. 10:1075-1088, Norton. (2005) Oncologist 10:370-381), yet these simplistic models greatly influenced the development of standards of cancer treatment that are still enforced today; that is, to use drugs in combination, and to use them in equally spaced cycles of equal intensity. While the prediction that tumor shrinkage is correlated with improved prognosis remains true, the prediction that giving conventional drugs long enough would lead to tumor eradication has turned out to be false (Norton (2006) Oncol. 4:36-37) Appreciation of a more complex kinetics, as described by Benjamin Gompertz and formalized as the Norton-Simon model, takes into account the dynamics of metastasis and the quantitative relationship between tumor burden and metastatic potential in its predictions. Thus, the concept of dose-dense chemotherapies emerged, which emphasized the optimal doses of drugs that cause regression of the tumor over shorter time intervals and favored sequential rather than combinatorial approaches ((Norton (2006); Fornier and Norton (2005) Breast Cancer Res. 7: 64-69). Subsequently, a number of clinical trials provided supportive evidence that giving drugs more densely made a significant difference in terms of optimizing cancer cell kill.

In the present studies, a variety of exemplary protocols for the targeted therapeutic vectors were designed for cancer patients. For example, in one study, an intra-patient dose escalation regimen by intravenous infusion of REXIN-G was given daily for 8-10 days. Completion of this regimen was followed by a one-week rest period for assessment of toxicity; after which, the maximum tolerated dose of REXIN-G was administered IV for another 8-10 days. If the patient did not develop a grade 3 or 4 adverse event related to REXIN-G during the treatment periods, the dose of REXIN-G was escalated as follows:

TABLE 1
Treatment Regimen

| | Dose | |
|---|---|---|
| Treatment Day | Level | Vector Dose/Day |
| Day 1-6 (Dose Escalation | I | 4.5 × 10⁹ Units |
| Regimen) | | |
| Day 7-8 | II | 9.0 × 10⁹ Units |
| Day 9-10 | III | 1.4 × 10¹⁰ Units |
| Day 18-27 (High Dose Regimen) | III | 1.4 × 10¹⁰ Units |

Based on the observed safety in the first two patients, a third patient with Stage IVB pancreatic cancer with numerous liver metastases was given a frontline treatment with intravenous REXIN-G for six days, followed by 8 weekly doses of gemcitabine at 1000 mg/m² in a second clinical protocol approved by the Philippine BFAD.

The introduction of pathotropic nanoparticles for targeted gene delivery enables a new and quantitative approach to treating metastatic cancer in a unique and strategic manner. The Calculus of Parity described herein represents an emergent paradigm that seeks to meet and to match a given tumor burden in a highly compressed period of time; in other words, a Dose-Dense Induction Regimen based quantitatively on best estimates of total tumor burden. The Calculus of Parity assumes from the outset, (i) that the therapeutic agent (in this case REXIN-G™) is adequately targeted such that physiological barriers including dilution, turbulence, flow, diffusion barriers, filtration, inactivation, and clearance are sufficiently counteracted such that a physiological performance coefficient (φ) or physiological multiplicity of infection (P-MOI) can be calculated, (ii) that the agent is effective at levels that do not confer restrictive dose-limiting toxicities, and (iii) that the agent is available in sufficiently high concentrations to allow for intravenous administration of the personalized doses without inducing volume overload. The physiological performance coefficient for cytocidal cyclin G1 constructs varies from 4 to 250, and depends in part on the titer of the drug (Gordon et al. (2000) Cancer Res. 60:3343-3347). To calculate the optimal dosage of the therapeutic targeted vectors, including REXIN-G, to be given each day, the following factors were taken into consideration: (1) the total tumor burden based on radiologic imaging studies, (2) the physiological performance coefficient (φ) of the system, which specifies the multiplicity of inducible gene transfer units needed per target cancer cell, and (3) the precise potency of the drug defined in terms of vector titer, which is expressed in colony forming units (U) per ml. One gene transfer unit is the equivalent of one colony forming unit. The Calculus of Parity predicts that tumor control can be achieved if the dose of the targeted vector administered is equivalent to the emergent tumor burden; yet the total dosage should be administered in as short a period of time as considered safely possible, in order to prevent catch-up tumor growth while allowing time for the reticuloendothelial system to eliminate the resulting tumor debris (Gordon et al. (2000) Cancer Res. 60:3343-3347).

The Calculus of Parity Equation:

$$Dose of Gene Therapy Drug Needed for Initial Tumor Control = \frac{Tumor Burden \times pMOI}{Potency of Drug}$$

The Calculus of Parity as Applied to Treatment with the Therapeutic Vector Particles: Where Tumor Burden is derived from the equation [the sum of the longest diameters (cm) of target lesions]×[1×10e9 cancer cells/cm]

Where φ or pMOI is an empiric number estimated from preclinical and clinical studies

For REXIN-G pMOI is 100

Where Potency is the number of colony forming units (U) per ml of drug solution.

For REXIN-G produced using the new constructs, pB-RVE and pdnG1/EREX, Potency ranges from 5×10e8 to 5×10e9 Units/ml

### Example

### REXIN-G Dose Calculation for a Patient with Metastatic Pancreatic Cancer

Where patient has a locally advanced tumor of dimensions of 2 cm×2 cm and 4 liver lesions, three of which measure 1 cm×1 cm, and the fourth measures 2 cm×2 cm

Tumor Burden(pancreas,liver)=(4 cm+(2 cm+2 cm+2 cm+4 cm))×1×10e9 cells/cm=14×10e9 cancer cells

Where the specific lot of REXIN-G has Potency of 1×10e9 U/ml

$$\begin{matrix}REXIN \text{-} G Dose ( ml ) = \frac{14 \times 10 e 9 cells \times 100 U \text{/} cell}{1 \times 10 e 9 U \text{/} ml} \\ = \frac{14 \times 10 e 11 U = 1400 ml}{1 \times 10 e 9 U \text{/} ml}\end{matrix}$$

### Example

### Calculation of the Number of REXIN-G Storage Units to Administer

To determine the number of REXIN-G storage units (e.g. glass vials, cryobags) needed for infusion, the total volume of the REXIN-G dose is divided by the standard volume of REXIN-G contained in a storage unit from the lot used. REXIN-G may be supplied in, for example, cryobags or glass vials in either 20 ml or 40 ml aliquots.

$$Number of REXIN \text{-} G storage units needed = \frac{Volume of REXIN \text{-} G dose}{Volume per storage unit}$$

With REXIN-G supplied as 40 ml aliquots the needed number of doses is: 1400 ml=35 storage units (40 ml each)

Three dosing schedules for different tumor burden were derived using the Calculus of Parity (see above).

| Estimated | | |
|---|---|---|
| Tumor Burden | | |
| by Calculus | | |
| of Parity | Initial/Induction (4 weeks) | Maintenance (6 months) |
| Small Tumor | 4.0 × 10e10 Units per day, | Repeat 2- to 4-week cycle |
| Burden | Mon-Fri with rest on | Re-calculate parity to |
| (<5 × 10e9 | week-ends × 4 weeks; | determine the cumulative |
| cancer cells) | 2 week rest period followed | dose to be given |
| | by tumor response | |
| | evaluation by CT, MRI or | |
| | PET scan | |
| Moderate | 8.0 × 10e10 Units per day, | Repeat 2- to 4-week cycle |
| Tumor Burden | Mon-Fri with rest period on | Re-calculate parity to |
| (5-10 × 10e9 | week-ends × 4 weeks; | determine the cumulative |
| cancer cells) | 2 week rest period followed | dose to be given |
| | by tumor response | |
| | evaluation by CT, MRI or | |
| | PET scan | |
| Large Tumor | 1.2 × 10e11 Units per day, | Repeat 2- to 4-week cycle |
| Burden | Mon-Fri with rest period on | Re-calculate parity to |
| (>10 × 10e9 | week-ends × 4 weeks; or | determine the cumulative |
| cancer cells) | 2.0 × 10e11 Units per day | dose to be given |
| | M-W-F for 4 weeks; | |
| | 2 week rest period followed | |
| | by tumor response | |
| | evaluation by CT, MRI or | |
| | PET scan | |

The advent of targeted therapies, including targeted gene therapy, is changing the way tumor responses to a cancer drug are being evaluated. The methods disclosed herein are especially useful in treating cancers or other disorders resistant to traditional therapies, e.g. resistant to chemotherapy, antibody-based therapies or other standard therapies. Induction of remission, enabling of surgical resection of the tumor, or prevention of recurrence of the cancer or other disorder are among the objective responses gained from use of the targeted delivery vectors. The methods described herein are especially useful in cancers or other disorders that are resistant to traditional therapies, e.g. resistant to chemotherapy, antibody-based therapies or other standard therapies. Accordingly, administration of the targeted delivery vectors may occur even after all standard therapies have failed or been less than successful.

Additionally, combination of the targeted delivery vectors with standard therapies (e.g. chemotherapeutic agent, a biologic agent, or radiotherapy prior to, contemporaneously with, or subsequent to the administration of the therapeutic viral particles) may also be used. Accordingly, combination of the targeted delivery vectors with primary, adjuvant or neoadjuvant anti-cancer therapies are contemplated as an embodiment of the present disclosure. As used herein, the terms “cancer treatment,” “cancer therapy,” “anti-cancer therapy” and the like encompasses treatments such as surgery, radiation therapy, administration of chemotherapeutic agents and combinations of any two or all of these methods. Combination treatments may occur sequentially or concurrently. Treatments, such as radiation therapy and/or chemotherapy, that is administered prior to surgery, are referred to as neoadjuvant therapy. Treatments, such as radiation therapy and/or chemotherapy, administered after surgery is referred to herein as adjuvant therapy. Examples of surgeries that may be used for cancer treatment include, but are not limited to radical prostatectomy, cryotherapy, mastectomy, lumpectomy, transurethral resection of the prostate, and the like.

Anti-cancer therapies include, but are not limited to, DNA damaging agents, topoisomerase inhibitors and mitotic inhibitors. Many chemotherapeutics are presently known in the art and can be used in combination with the targeted delivery vectors described herein. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

A principle in cancer therapy has been that the therapeutic benefit gained from a prospective chemotherapeutic agent must outweigh the risk of serious or fatal systemic toxicity induced by the drug candidate. To this end, the Response Evaluation Criteria in Solid Tumors (RECIST) was developed by the National Cancer Institute (NCI), Bethesda Md., USA, and has been employed by most, if not all, academic institutions as the universal standard for tumor response evaluations (Therasse et al., (2000) J. Nat'l. Cancer Inst. 92:205-216). Specifically, an objective tumor response (OTR) has, until recently, been considered the golden standard of success in evaluating cancer therapy for solid tumors. An OTR consists of at least a 30% reduction in the size of target lesions and/or complete disappearance of metastatic foci or non-target lesions. However, many biologic response modifiers of cancer are, in fact, not associated with tumor shrinkage, but have been shown to prolong progression-free survival (PFS), and overall survival (OS) (Abeloff, (2006) Oncol. News Int'l. 15:2-16). Hence, the response to effective biologic agents is often physiologic and RECIST may no longer be the appropriate standard for evaluation of tumor response to biologic therapies. Thus, alternative surrogate endpoints such as measurements of tumor density (an index of necrosis), blood flow and glucose utilization in tumors, and other refinements of imaging methods used to evaluate the mechanisms of tumor response are called for.

Understanding the disease process, as well as the intended mechanisms of action of the proposed intervention, is, therefore, critical in predicting the effect of the treatment on a given clinical endpoint. In the case of tumor responses to the targeted therapeutic vectors, wherein the primary mechanism of action is the induction of apoptosis in proliferative tumor cells and attendant angiogenic vasculature, necrosis and cystic changes within the tumor often occur. This is due to the targeted disruption of a tumor's blood supply which starves the tumor, resulting in subsequent necrosis within the tumor. For example, in tumors of REXIN-G-treated patients, wherein apoptosis is a predominant feature, the tumors simply shrink and disappear in follow-up imaging studies. However, in tumors wherein necrosis is a prominent feature, the size of the tumors may actually become larger after REXIN-G treatment, due to the inflammatory reaction evoked by the necrotic tumor and cystic conversion of the tumor. In this case, an increase in the size of tumor nodules on CT scan, PET scan or MRI does not necessarily indicate disease progression. Therefore, additional concomitant evaluations that reflect the histological quality of the treated tumors may be used to more accurately determine the extent of necrosis or cystic changes induced by treatment, and accordingly monitor progress of the therapeutic retroviral vector particle therapy. For CT scans tumor density measurement in Hounsfield Units (HU) is an accurate and reproducible index of the extent of tumor necrosis. A progressive reduction in the density of target lesions (decrease in HU) indicates a positive treatment effect. For PET scans a progressive reduction in standard uptake value (SUV) in target lesions indicates decreased tumor activity and positive treatment effect. For biopsied tumor the presence of apoptosis, necrosis, reactive fibrosis and tumor infiltrating lymphocytes (TILS) indicate a positive treatment effect. In addition, PET criteria (metabolic activity), and CHOI criteria (tumor density), as well as RECIST (size only) may also be used to determine progress of the targeted therapeutic vector therapy program.

The administration of retroviral vectors may elicit the production of vector neutralizing antibodies in the recipient, thereby hampering further treatment. (Halbert et al. (2006) Hum. Gene Ther. 17(4):440-447). It is known, however, in the art, that the induction of neutralizing antibody production can be blocked by the immunosuppressive treatment given around the time of vector administration. Such immunosuppressive treatments include drugs (cyclophosphamide, FK506), cytokines (interferon-gamma, interleukin-12) and monoclonal antibodies (anti-CD4, anti-pgp39, CTLA4-Ig) (Potter and Chang, (1999) Ann. N.Y. Acad. Sci. 875:159-174). Furthermore, neutralizing antibodies may be removed by extracorporeal immunoadsorption (Nilsson et al. (1990) Clin. Exp. Immunol. 82(3)440-444). Neutralizing antibodies can also be depleted in vivo by the administration of larger doses of vector. The REXIN-G vector has low immunogenicity and to date, vector neutralizing antibodies have not been detected in the serum of patients over a 6 month follow-up period.

Kits

Also provided are kits or drug delivery systems comprising the compositions for use in the methods described herein. All the essential materials and reagents required for administration of the targeted retroviral particle may be assembled in a kit (e.g., packaging cell construct or cell line, cytokine expression vector). The components of the kit may be provided in a variety of formulations as described above. The one or more targeted retroviral particle may be formulated with one or more agents (e.g., a chemotherapeutic agent) into a single pharmaceutically acceptable composition or separate pharmaceutically acceptable compositions.

The components of these kits or drug delivery systems may also be provided in dried or lyophilized forms. When reagents or components are provided as a dried form, reconstitution generally is by the addition of a suitable solvent, which may also be provided in another container means. The kits of the invention may also comprise instructions regarding the dosage and or administration information for the targeted retroviral particle. The kits or drug delivery systems of the present invention also will typically include a means for containing the vials in close confinement for commercial sale such as, e.g., injection or blow-molded plastic containers into which the desired vials are retained. Irrespective of the number or type of containers, the kits may also comprise, or be packaged with, an instrument for assisting with the injection/administration or placement of the ultimate complex composition within the body of a subject. Such an instrument may be an applicator, inhalant, syringe, pipette, forceps, measured spoon, eye dropper or any such medically approved delivery vehicle.

In another embodiment, a method for conducting a gene therapy business is provided. The method includes generating targeted delivery vectors and establishing a bank of vectors by harvesting and suspending the vector particles in a solution of suitable medium and storing the suspension. The method further includes providing the particles, and instructions for use of the particles, to a physician or health care provider for administration to a subject (patient) in need thereof. Such instructions for use of the vector can include the exemplary treatment regimen provided in Table 1. The method optionally includes billing the patient or the patient's insurance provider.

In yet another embodiment, a method for conducting a gene therapy business, including providing kits disclosed herein to a physician or health care provider, is provided.

The following examples are included for illustrative purposes only and are not intended to limit the scope of the invention. The specific methods exemplified can be practiced with other species. The examples are intended to exemplify generic processes.

### EXAMPLES

### Example 1

### Constructs

The plasmid pBv1/CAEP contains coding sequences of the 4070A amphotropic envelope protein (GenBank accession number: M33469), that have been modified to incorporate an integral gain of collagen-binding function (Hall et al., Human Gene Therapy, 8:2183-2192, 1997). The parent expression plasmid, pCAE (Morgan et al., Journal of Virology, 67:4712-4721, 1967) was provided by the USC Gene Therapy Laboratories. This pCAE plasmid was modified by insertion of a Pst I site (gct gca gga, encoding the amino acids AAG) near the N-terminus of the mature protein between the coding sequences of amino acids 6 and 7 (pCAEP). A synthetic oligonucleotide duplex (gga cat gta gga tgg aga gaa cca tca ttc atg gct ctg tca gct gca) (SEQ ID NO:5), encoding the amino acids GHVGWREPSFMALSAA (SEQ ID NO:1), a minimal collagen-binding decapeptide (in bold) derived from the D2 domain of bovine von Willebrand Factor (Hall et al., Human Gene Therapy, 11:983-993, 2000) and flanked by strategic linkers (underlined), was cloned into this unique Pst I site to produce pBv1/CAEP.

The expression of the chimeric envelope protein in 293T producer cells is driven by the strong CMV i.e. promoter. The chimeric envelope is processed correctly and incorporated stably into retroviral particles, which exhibit the gain-of-function phenotype without appreciable loss of infectious titer. Correct orientation of the collagen-binding domain was confirmed by DNA sequence analysis, and plasmid quality control was confirmed by restriction digestion Pst I, which linearizes the plasmid and releases the collagen-binding domain.

Further improvements to the original plasmid pBv1/CAEP were made to reduce the potential to generate replication-competent retrovirus (RCR) during REXIN-G™ production. The vector pBv1/CAEP contains 38 base pairs of untranslated sequences upstream of the Moloney Envelope ATG start codon. This vector also contains 76 base pairs of untranslated sequences downstream of the Moloney Envelope stop codon. Both of these untranslated sequences (38+76=114 base pairs) were eliminated by using the polymerase chain reaction technique to amplify only the Moloney Envelope open reading frame sequences from the ATG start codon to the TGA stop codon. The following sets of primers were used:

NewEnvF1
(SEQID NO: 6)
5′ ATGCGGCCGCCCACC [symbol] GCGCGTTCAACGCTCTCAAAACCCCCTCAA
GATA 3′
NewEnvR1
(SEQ ID NO: 7)
5′ CCTCTAGATTA [symbol] TGGCTCGTACTCTATGGGTTTTAGCTGG 3′

pBV1/CAEP was used as the template for the PCR reaction to insure that the unique von Willebrand collagen binding site (GHVGWREPSFMALSAA) (SEQ ID NO:1) would be properly copied into the new open reading frame only Envelope PCR product. The proper 2037 bp pair PCR product was produced and ligated into a pCR2 cloning vector and sequenced to insure 100% sequence conformity to expected sequence. This sequenced Moloney Envelope open reading frame only gene was excised from the pCR2 plasmid backbone and subcloned into the ultra high expression plasmid pHCMV form Genelantis to produce the new plasmid, pB-RVE.

This plasmid was tested in a number of different titer assays and found to its strength had increased such that it was now optimal to use 3-5 times less of it by quantity in a transfection in to 293T cells along with pCgpn and pE-REX to achieve similar titers. This implies that the pB-RVE plasmid is 3-5 times stronger than the corresponding pBV1/CAEP plasmid in producing functional envelope protein. However, if the same amount of pB-RVE plasmid is used as the normal amount pBV1/CAEP, far less titer would be produced. This result stresses the importance of conducting a complete set of plasmid ratio studies to obtain the optimal ratio for highest titer. In some circumstances, over expression of any one of the three plasmid component genes can disrupt a delicate balance of viral parts during assembly and processing and can cause inhibitory effects as noted in lower titers. We chose to use 3-5 times less pB-RVE than pBV1/CAEP to achieve a similar high titer and gain the advantage with this plasmid of using that much less of it during GMP retroviral production. This high level expression effect is most like due to the fact that the Envelope gene is expressed from a CMV promoter enhancer in tandem with a CMV Intron. The combination is advertised to be 3-5 times stronger than if just expressed from a CMV promoter as is the case for the pBV1/CAEP plasmid.

The plasmid pCgpn contains the MoMuLV gag-pol coding sequences (GenBank Accession number 331934), initially derived from proviral clone 3PO as pGag-pol-gpt, (Markowitz et al., Journal of Virology, 62:1120-1124, 1988) exhibiting a 134-base-pair deletion of the Ψ packaging signal and a truncation of env coding sequences. The construct was provided as an EcoRI fragment in pCgp in which the 5′ EcoRI site corresponds to the XmaIII site upstream of Gag and the 3′ EcoRI site was added adjacent to the ScaI site in env. The EcoRI fragment was excised from pCgp and ligated into the pcDNA3.1+ expression vector (Invitrogen) at the unique EcoRI cloning site.

Correct orientation was confirmed by restriction digestion with Sail and the insert was further characterized by digestion with EcoRI and HindIII. Both the 5′ and 3′ sequences of the gag-pol insert were confirmed by DNA sequence analysis utilizing the T7 promoter binding site primer (S1) and the pcDNA3.1/BGH reverse priming site (AS1), respectively. The resulting plasmid, designated pCgpn, encodes the gag-pol polyprotein driven by the strong CMV promoter and a neomycin resistance gene driven by the SV40 early promoter. The presence of an SV40 ori in this plasmid enables episomal replication in cell lines that express the SV40 large T antigen (i.e., 293T producer cells).

The following describes the construction of the plasmid bearing the pdnG1/C-REX retroviral expression vector which contains the dominant negative cyclin G1 construct (dnG1). The plasmid is enhanced for production of vectors of high infectious titer by transient transfection protocols. The cDNA sequences (472-1098 plus stop codon) encoding aa 41 to 249 of human cyclin G1 (CYCG1, Wu et al., Oncology Reports, 1:705-11, 1994; accession number U47413) were generated from a full length cyclin G1 template by PCR, incorporating Not I/Sal I overhangs. The N-terminal deletion mutant construct was cloned initially into a TA cloning vector (Invitrogen), followed by Not I/Sal I digestion and ligation of the purified insert into a Not I/Sal I digested pG1XSvNa retroviral expression vector (Genetic Therapy, Inc.) to produce the pdnG1SvNa vector complete with 5′ and 3′ long terminal repeat (LTR) sequences and a Ψ retroviral packaging sequence.

A CMV i.e. promoter-enhancer was prepared by PCR from a CMV-driven pIRES template (Clontech), incorporating Sac II overhangs, and cloned into the unique Sac II site of pdnG1SvNa upstream of the 5′ LTR. The neomycin resistance gene, which facilitates determination of vector titer, is driven by the Sv40 e.p. with its nested ori. The inclusion of the strong CMV promoter, in addition to the Sv40 ori, facilitate high titer retroviral vector production in 293T cells expressing the large T antigen (Soneoka et al., Nucleic Acid Research, 23:628-633, 1995). Correct orientation and sequence of the CMV promoter was confirmed by restriction digestion and DNA sequence analysis, as was the dnG1 coding sequences. Plasmid identity and quality control is confirmed by digestion with Sac II (which releases the 750 bp CMV promoter) and Bgl II (which cuts at a unique site within the dnG1 construct).

Multiple GMP retroviral productions using pdnG1/C-REX and pBV1-CAEP have proven to be safe and RCR-free. The 4^{th} and 5^{th} generation MLV-based retroviral vectors and vector production methodologies; i.e., split genome designs, have yielded consistent production qualities without generating RCR under standard GMP conditions (Sheridan et al., 2000; Merten, 2004). However, we, as well as others have discerned that all available vector constructs contain a significant number of residual gag-pol sequences that potentially overlap with 5′ DNA sequences contained in the respective gag-pol plasmid construct (Yu et al., 2000); and that these significant areas of overlap could become problematic when vector production is eventually scaled-up to commercial volumes with larger cell numbers and corresponding plasmid concentrations.

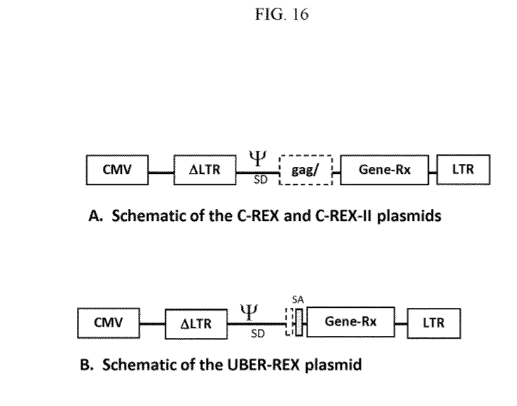

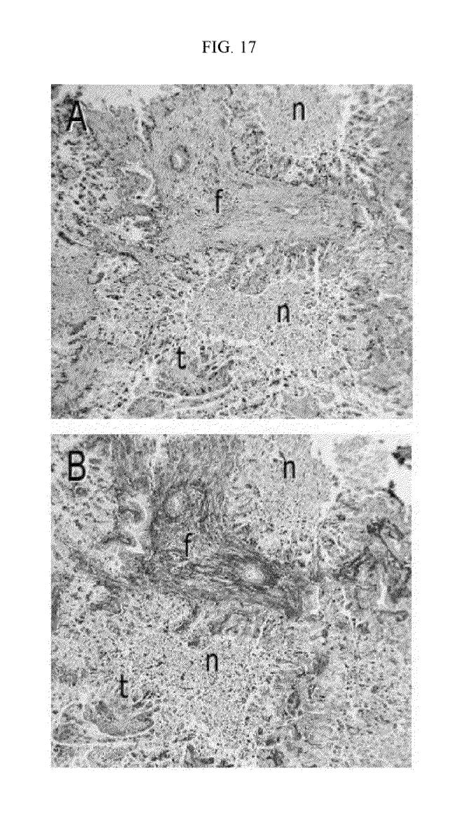

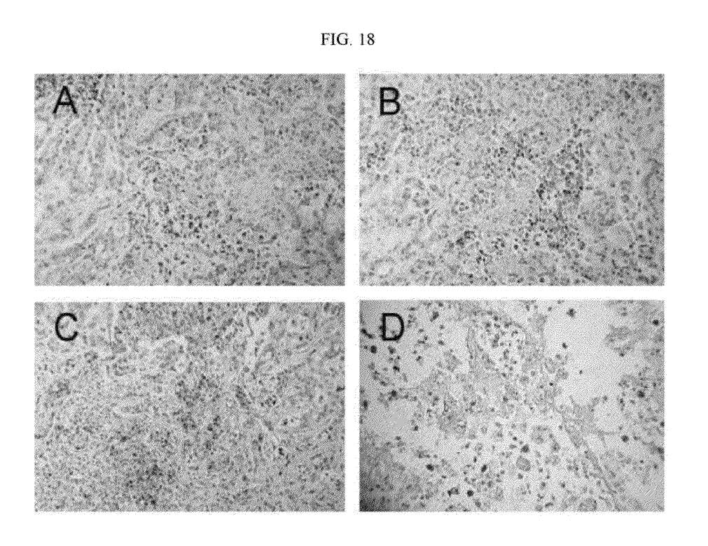

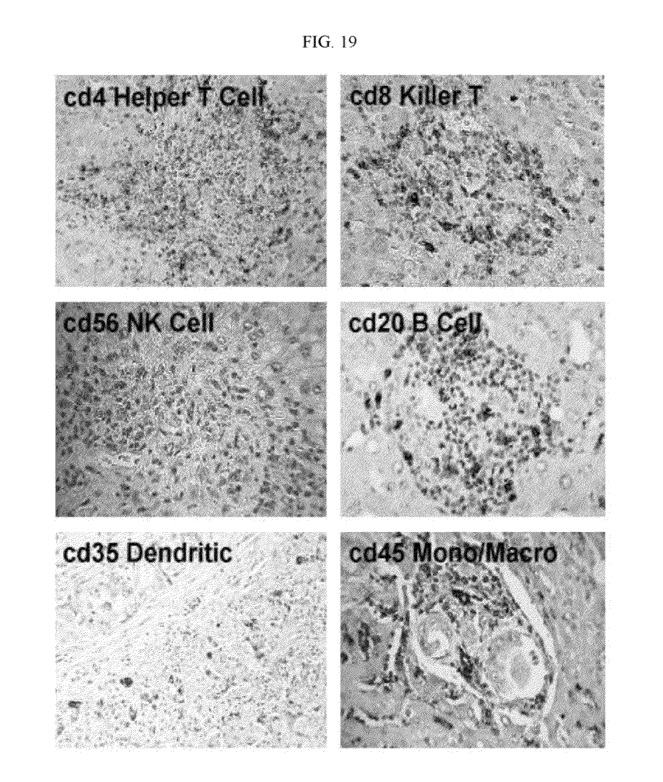

With these considerations in mind, we elected to remove 487 base pairs of residual gag-pol sequences from the parent pdnG1/C-REX vector by restriction digest and PCR cloning (pdnG1/C-ΔREX) followed by the insertion of a synthetic 97 bp envelop splice acceptor site (ESA) (Lazo et al., (1987) J. Virol. 61(6): 2038-41) which served to offset detriments in terms of packaging (titer) and gene expression (potency). These resulting safety modifications of pdnG1/C-REX have resulted in the generation of pdnG1/UBER-REX, which encodes and expresses exactly the same transgenes (dnG1 and neo) without 487 base pairs of GAG, and which now replaces the former plasmid in the production of REXIN-G. A schematic comparison between the C-REX and C-REXII plasmids, and the UBER-REX plasmid is shown in FIG. 16.

The combination of the pB-RVE, pCgpn, pdnG1/UBER plasmids at exact ratios and under highly controlled and optimized manufacturing conditions yield a clinical vector product without RCR and the highest unconcentrated GMP final product retroviral titer ever reported, >5×10⁹ cfu/mL.

### Example 2

### REXIN-G

The final product, Mx-dnG1 (REXIN-G™), is a matrix (collagen)-targeted retroviral vector encoding a N-terminal deletion mutant human cyclin G1 construct under the control of a hybrid LTR/CMV promoter. The vector also contains the neomycin resistance gene which is driven by the SV40 early promoter.

The Mx-dnG1 vector is produced by transient co-transfection with 3 plasmids of 293T (human embryonic kidney 293 cells transformed with SV40 large T antigen) cells obtained from a fully validated master cell bank.

The components of the transfection system includes the pdnG1/C-REX therapeutic plasmid construct which contains the deletion mutant of the human cyclin G1 gene encoding a.a. 41 to 249 driven by the CMV immediate early promoter, packaging sequences, and the bacterial neomycin resistance gene under the control of an internal SV40 early promoter. The truncated cyclin G1 gene was initially cloned into a TA cloning vector (Invitrogen), followed by Not I/Sal I digestion and ligation of the purified insert into a Not I/Sal I digested pG1XSvNa retroviral expression vector (provided by Genetic Therapy, Inc., Gaithersburg, Md.) to produce the pdnG1SvNa vector complete with 5′ and 3′ LTR sequences and a Ψ sequence. The CMV i.e. promoter-enhancer was prepared by PCR from a CMV-driven pIRES template (Clontech), incorporating Sac II overhangs, and cloned into the unique SacII site of pdnG1SvNa upstream of the 5′LTR.

The use of the plasmid, pdnG1/C-REX, was replaced by pdnG1/UBER-REX, a next generation plasmid that encodes and expresses exactly the same transgenes (dnG1 and neo) without 487 base pairs of GAG found in the original pdnG1/C-REX.

The system further includes the Mx (Bv1/pCAEP) envelope plasmid containing a CMV-driven modified amphotropic 4070A envelope protein wherein a collagen-binding peptide was inserted into an engineered Pst I site between a.a. 6 and 7 of the N terminal region of the 4070A envelope.

The use of the Mx (Bv1/pCAEP) envelope plasmid was replaced by pB-RVE, an improved plasmid that eliminates 114 bp of extraneous retroviral sequences that potentially overlap with native untranslated (UTR) sequences.

The system also includes the pCgpn plasmid which contains the MLV gag-pol elements driven by the CMV immediate early promoter. It is derived from clone 3PO as pGag-pol-gpt. The vector backbone is a pcDNA3.1+ from Invitrogen. Polyadnylation signal and transcription termination sequences from bovine growth hormone enhance RNA stability. An SV40 ori is featured along with the e.p. for episomal replication and vector rescue in cell lines expressing SV40 target T antigen.

The plasmids have been analyzed by restriction endonuclease digestion and the cell line consists of a DMEM base supplemented with 4 grams per liter glucose, 3 grams per liter sodium bicarbonate, and 10% gamma irradiated fetal bovine serum (Biowhittaker). The serum was obtained from USA sources, and has been tested free of bovine viruses in compliance with USDA regulations. The budding of the retroviral particles is enhanced by induction with sodium butyrate. The resulting retroviral particles are processed solely by passing the supernatant through a 0.45 micron filter or concentrated using a tangential flow/diafiltration method. The retroviral particles are Type C retrovirus in appearance. Retroviral particles will be harvested and suspended in a solution of 95% DMEM medium and 1.2% human serum albumin. This formulation is stored in aliquots of 150 ml in a 500 ml cryobag and kept frozen at −70 to −86° C. until used.

For REXIN-G™ produced with the improved pB-RVE and pdnG1/UBER-REX plasmids, the production, suspension, and collection of therapeutic nanoparticles are performed in the absence of bovine serum in a final formulation of proprietary medium, which is processed by sequential clarification, filtration and final fill into cryobags using a sterile closed loop system. The resulting C-type retroviral particles, with an average diameter of 100 nanometers, are devoid of all viral genes, and are fully replication defective. The titers of the clinical lots range from 3×10e7 to 5×10e9 colony forming units (U)/ml, and each lot is validated for requisite purity and biological potency.

Preparation of the Mx-dnG1 vector for patient administration consists of thawing the vector in the vector bag in a 37° C. 80% ethanol bath. Each vector bag will be thawed one hour prior to infusion into the patient, treated with Pulmozyme (10 U/ml), and immediately infused within 1-3 hours.

Processed clinical-grade REXIN-G™ produced with the improved pB-RVE and pdnG1/UBER-REX plasmids is sealed in cryobags that are stored in a −70±10° C. freezer prior to shipment. Each lot of validated and released cryobags containing the REXIN-G™ vector is shipped on dry ice to the Clinical Site where the vector is stored in a −70±10° C. freezer until used. Fifteen minutes before intravenous infusion, the vector is rapidly thawed in a 32-37° C. water bath and immediately infused or transported on ice in a dedicated tray or cooler to the patient's room or clinical site for immediate use. Patients receive the infusion of REXIN-G™ via a peripheral vein, a central IV line, or a hepatic artery. Various dosing regimens were used, as described in clinical studies A, B and C (below); however, a maximum volume of 8 ml/kg/dose is given once a day. Each bag of REXIN-G™ is infused over 10-30 minutes at a rate of 4 ml/min.

### Example 3

### Therapeutic Efficacy of the Mx-dnG1 Vector

The efficacy of Mx-dnG1 in inhibiting cancer cell proliferation in vitro, and in arresting tumor growth in vivo in a nude mouse model of liver metastasis, was tested. A human undifferentiated cancer cell line of pancreatic origin was selected as the prototype of metastatic cancer. Retroviral transduction efficiency in these cancer cells was excellent, ranging from 26% to 85%, depending on the multiplicity of infection (4 and 250 respectively). For selection of a therapeutic gene, cell proliferation studies were conducted in transduced cells using vectors bearing various cyclin G1 constructs. Under standard conditions, the Mx-dnG1 vector consistently exhibited the greatest anti-proliferative effect, concomitant with the appearance of immunoreactive cyclin G1 at the region of 20 kDa, representing the dnG1 protein. Based on these results, the Mx-dnG1 vector was selected for subsequent in vivo efficacy studies.

To assess the performance of Mx-dnG1 in vivo, a nude mouse model of liver metastasis was established by infusion of 7×10⁵ human pancreatic cancer cells into the portal vein via an indwelling catheter that was kept in place for 14 days. Vector infusions were started three days later, consisting of 200 ml/day of either Mx-dnG1 (REXIN-G; titer: 9.5×10⁸ cfu/ml) or PBS saline control for a total of 9 days. The mice were sacrificed one day after completion of the vector infusions.

Histologic and immunocytochemical evaluation of metastatic tumor foci from mice treated with either PBS or low dose Mx-dnG1 was performed and evaluated with an Optimas imaging system. The human cyclin G1 protein was highly expressed in metastatic tumor foci, as evidenced by enhanced cyclin G1 nuclear immunoreactivity (brown-staining material) in the PBS-treated animals, and in the residual tumor foci of Mx-dnG1 vector-treated animals. Histologic examination of liver sections from control animals revealed substantial tumor foci with attendant areas of angiogenesis and stroma formation; the epithelial components stained positive for cytokeratin and associated tumor stromal/endothelial cells stained positive for vimentin and FLK receptor. In contrast, the mean size of tumor foci in the low dose Mx-dnG1-treated animals was significantly reduced compared to PBS controls (p=0.001), simultaneously revealing a focal increase in the density of apoptotic nuclei compared to the PBS control group. Further, infiltration by PAS+, CD68+ and hemosiderin-laden macrophages was observed in the residual tumor foci of Mx-dnG1-treated animals, suggesting active clearance of degenerating tumor cells and tumor debris by the hepatic reticuloendothelial system. Taken together, these findings demonstrate the anti-tumor efficacy in vivo of a targeted injectable retroviral vector bearing a cytocidal cell cycle control gene, and represent a definitive advance in the development of targeted injectable vectors for metastatic cancer.

In a subcutaneous human pancreatic cancer model in nude mice, we demonstrated that intravenous (IV) infusion of Mx-dnG1 enhanced gene delivery and arrested growth of subcutaneous tumors when compared to the non-targeted CAE-dnG1 vector (p=0.014), a control matrix-targeted vector bearing a marker gene (Mx-nBg; p=0.004) and PBS control (p=0.001). Enhanced vector penetration and transduction of tumor nodules (35.7+S.D.1.4%) correlated with therapeutic efficacy without associated systemic toxicity. Kaplan-Meier survival studies were also conducted in mice treated with PBS placebo, the non-targeted CAE-dnG1 vector and Mx-dnG1 vector. Using the Tarone logrank test, the over-all p value for comparing all three groups simultaneously was 0.003, with a trend that was significant to a level of 0.004, indicating that the probability of long term control of tumor growth was significantly greater with targeted Mx-dnG1 vector than with the non-targeted CAE-dnG1 vector or PBS placebo. Taken together, the present study demonstrates that Mx-dnG1, deployed by peripheral vein injection (i) accumulated in angiogenic tumor vasculature within one hour, (ii) transduced tumor cells with high level efficiency, and (iii) enhanced therapeutic gene delivery and long term efficacy without eliciting appreciable toxicity.

### Example 4

### Pharmacology/Toxicology Studies

Matrix-targeted injectable retroviral vectors incorporating peptides that target extracellular matrix components (e.g. collagen) have been demonstrated to enhance therapeutic gene delivery in vivo. Additional data are presented using two mouse models of cancer and two matrix-targeted MLV-based retroviral vectors bearing a cytocidal/cytostatic dominant negative cyclin G1 construct (designated Mx-dnG1 and MxV-dnG1). Both Mx-dnG1 and MxV-dnG1 are amphotropic 4070A MLV-based retroviral vectors displaying a matrix (collagen)-targeting motif for targeting areas of pathology. The only difference between the two vectors is that MxV-dnG1 is pseudotyped with a vesicular stomatitis virus G protein.

In the subcutaneous human cancer xenograft model, 1×10⁷ human MiaPaca2 pancreatic cancer cells (prototype for metastatic gastrointestinal cancer) were implanted subcutaneously into flank of nude mice. Six days later, 200 μl Mx-dnG1 vector was injected directly into the tail vein daily for one or two 10-day treatment cycles (Total vector dose: 5.6×10⁷ [n=6] or 1.6×10⁸ cfu [n=4] respectively). In the nude mouse model of liver metastasis, 7×10⁵ MiaPaca2 cells were injected through the portal vein via an indwelling catheter which was kept in place for 10-14 days. 200 ml of MxV-dnG1 vector was infused over 10 min daily for 6 or 9 days (Total vector dose: 4.8×10⁶ [n=3] or 1.1×10⁹ cfu dose [n=4] respectively) starting three days after infusion of tumor cells. For biodistribution studies, a TaqMan™ based assay was developed to detect the G1XSvNa-based vector containing SV40 and Neomycin (Neo) gene sequences into mouse genomic DNA background (Althea Technologies, San Diego, Calif., USA). The assay detects a 95 nt amplicon (nts. 1779-1874 of the G1XSvNa plasmid vector) in which the fluorescently labeled probe overlaps the 3′ portion of the SV40 gene and the 5′ portion of the neomycin phosphotransferase resistance (Neor) gene.

There was no vector related mortality or morbidity observed with either the Mx-dnG1 or MxV-dnG1 vector. Low level positive signals were detected in the liver, lung and spleen of both low dose and high dose vector-treated animals. No PCR signal was detected in the testes, brain or heart of vector-treated animals. Histopathologic examination revealed portal vein phlebitis, pyelonephritis with focal myocarditis in two animals with indwelling catheters and no antibiotic prophylaxis. No other pathology was noted in non-target organs of Mx-dnG1- or MxV-dnG1-treated mice. Serum chemistry profiles revealed mild elevations in ALT and AST in the Mx-dnG1-treated animals compared to PBS controls. However, the levels were within normal limits for mice. No vector neutralizing antibodies were detected in the sera of vector-treated animals in a 7-week follow-up period.

The preclinical findings noted above confirm that intravenous infusion of Mx-dnG1 in two nude mouse models of human pancreatic cancer showed no appreciable damage to neighboring normal tissues nor systemic side effects. The method of targeted gene delivery via intravenous infusion offers several clinically relevant advantages. Infusion into the venous system will allow treatment of the tumor as well as occult foci of tumor. It is believed that the higher mitotic rate observed in dividing tumor cells will result in a higher transduction efficiency in tumors, while sparing hepatocytes and other normal tissues. Therefore, we propose a human clinical research protocol using intravenously administered Mx-dnG1 vector for the treatment of locally advanced or metastatic pancreatic cancer and other solid tumors refractory to standard chemotherapy.

### Example 5

### Clinical Studies

The objectives of the study were (1) to determine the dose-limiting toxicity and maximum tolerated dose (safety) of successive intravenous infusions of REXIN-G, and (2) to assess potential anti-tumor responses. The protocol was designed for end-stage cancer patients with an estimated survival time of at least 3 months. Three patients with Stage IV pancreatic cancer who were considered refractory to standard chemotherapy by their medical oncologists were invited to participate in the compassionate use protocol using REXIN-G as approved by the Philippine Bureau of Food and Drugs. An intrapatient dose escalation regimen by intravenous infusion of REXIN-G was given daily for 8-10 days. Completion of this regimen was followed by a one-week evaluation period for dose limiting toxicity; after which, the maximum tolerated dose of REXIN-G was administered for another 8-10 days. If the patient did not develop a grade 3 or 4 adverse event related to REXIN-G during the observation period, the dose of REXIN-G was escalated as shown in Table 1 (supra).

Tumor response was evaluated by serial determinations of the tumor volume using the formula: width²×length×0.52 as measured by calipers, or by radiologic imaging (MRI or CT scan).

Patient #1, a 47 year-old Filipino female was diagnosed, by histologic examination of biopsied tumor tissue and staging studies, to have localized adenocarcinoma of the pancreatic head. She underwent a Whipples surgical procedure which included complete resection of the primary tumor. This was followed by single agent gemcitabine weekly for 7 doses, but chemotherapy was discontinued due to unacceptable toxicity. Several months later, a follow-up MRI showed recurrence of the primary tumor with metastatic spread to both the supraclavicular and abdominal lymph nodes. In compliance with the clinical protocol, the patient received two 10-day treatment cycles of REXIN-G for a cumulative dose of 2.1×10e11 Units over 28 days, with an interim rest period of one week. In the absence of systemic toxicity, the patient received an additional 10-day treatment cycle for a total cumulative dose of 3×10e11 Units.

The sizes of two superficial supraclavicular lymph nodes were measured manually using calipers. A progressive decrease in the tumor volumes of the supraclavicular lymph nodes was observed, reaching 33% and 62% reductions in tumor size, respectively, by the end of treatment cycle #2 on Day 28 (Table 2).

TABLE 2
Patient # 1 Caliper Measurements of Supraclavicular Lymph Nodes

| | | | | % Reduction |
|---|---|---|---|---|
| | Caliper | | Tumor Volume* | in Size from |
| Date | Measurement cm | | cm³ | Start of REXIN-G Rx |
| Day 1 | LN1 | 1.9 × 2.1 | 3.9 | |
| | LN2 | 1.5 × 1.8 | 2.1 | |
| Day 26 | LN1 | 1.8 × 1.8 | 3.0 | 23 |
| | LN2 | 1.3 × 1.3 | 1.1 | 48 |
| Day 27 | LN1 | 1.7 × 1.7 | 2.6 | 33 |
| | LN2 | 1.15 × 1.15 | 0.8 | 62 |

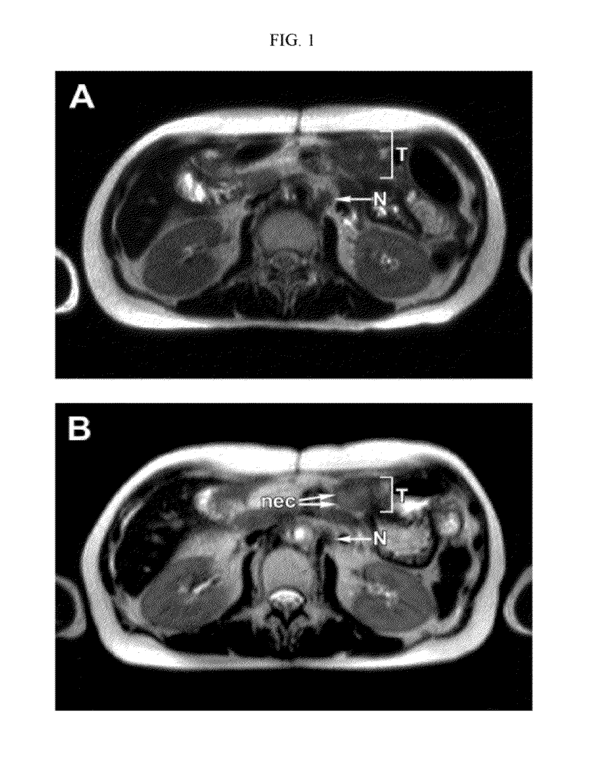

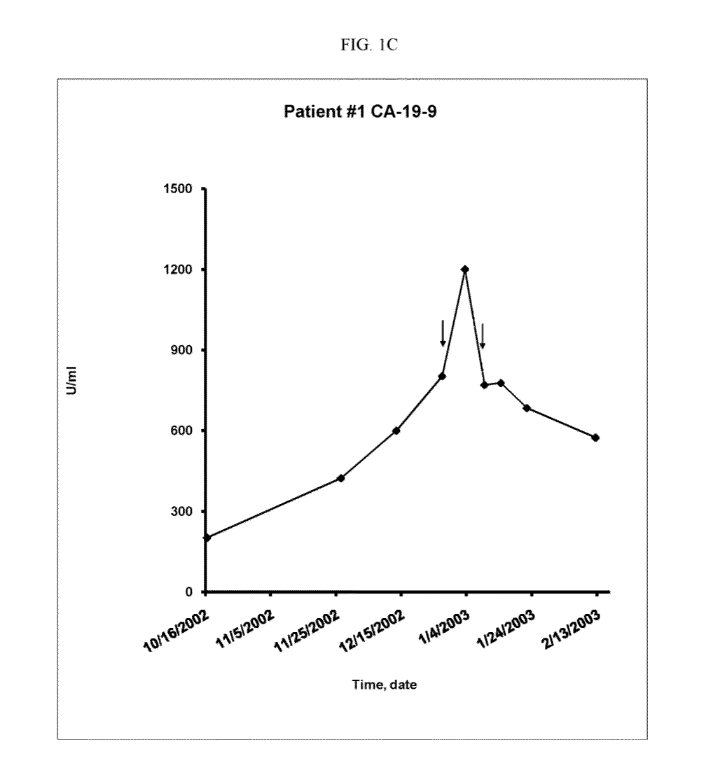

Follow-up abdominal MRI revealed (i) no new areas of tumor metastasis, (ii) discernable areas of central necrosis, involving 40-50% of the primary tumor, and (iii) a significant decrease in the size of the para-aortic abdominal lymph node (FIG. 1A-B). On Day 54, a follow-up MRI showed no interval change in the size of the primary tumor. Consistent with these findings, a progressive decrease in CA19-9 serum levels (from a peak of 1200 to a low of 584 U/ml) were noted, amounting to a 50% reduction in CA19-9 levels on Day 54 (FIG. 1C). However, a follow-up CT scan on Day 101 showed a significant increase in the size of the primary tumor and the supraclavicular lymph nodes. The patient refused further chemotherapy until Day 175 when the patient agreed to receive weekly gemcitabine, 1000 mg/m2. By RECIST criteria, Patient #1 is alive with progressive disease on Day 189 follow-up, 6.75 months from the start of REXIN-G infusions, 11 months from the time of tumor recurrence, and 20 months from the time of initial diagnosis.

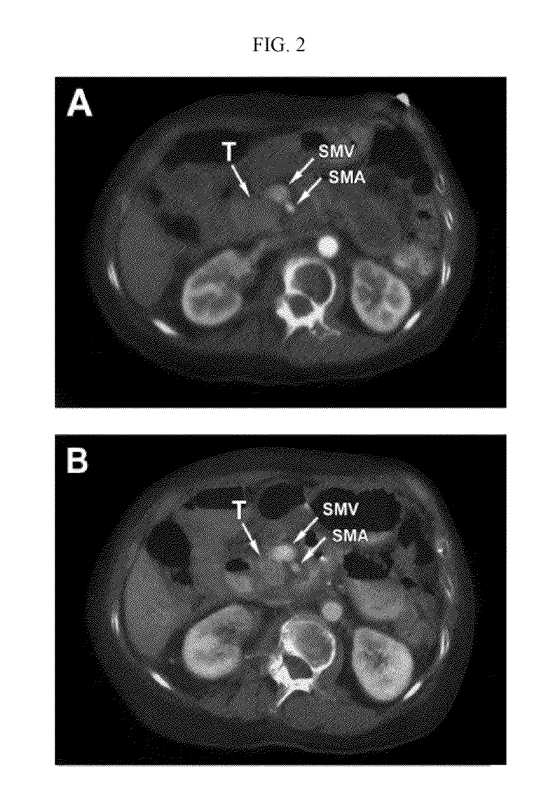

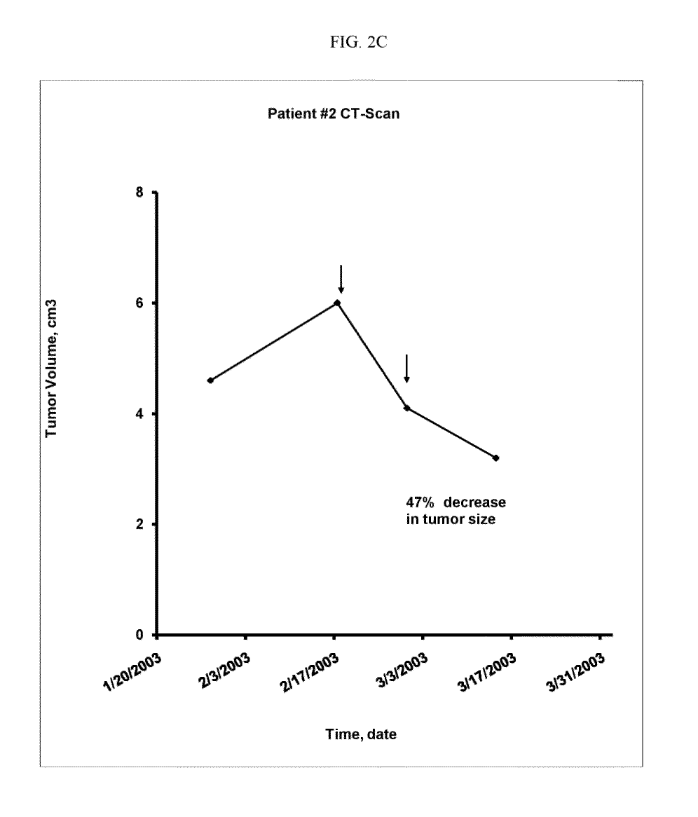

Patient #2, a 56 year-old Filipino female was diagnosed to have Stage IVA locally advanced and non-resectable carcinoma of the pancreatic head, by cytologic examination of biliary brushings. Exploratory laparotomy revealed that the tumor was wrapped around the portal vein and encroached in close proximity to the superior mesenteric artery and vein. She had received external beam radiation therapy with 5-fluorouracil, and further received single agent gemcitabine weekly for 8 doses, followed by monthly maintenance doses. However, a progressive rise in CA19-9 serum levels was noted and a follow-up CT scan revealed that the tumor had increased in size (FIG. 2A). The patient received two treatment cycles of REXIN-G as daily intravenous infusions for a total cumulative dose of 1.8×10¹¹ Units. Results: Serial abdominal CT scans showed a significant decrease in tumor volume from 6.0 cm³ at the beginning of REXIN-G infusions to 3.2 cm³, at the end of the treatment, amounting to a 47% decrease in tumor size on Day 28 (FIG. 2A-C). Follow-up CT scan on Day 103 showed no interval change in the size of the tumor, after which the patient was maintained on monthly gemcitabine. By RECIST criteria, Patient #2 is alive, asymptomatic with stable disease on Day 154 follow-up, 5.5 months from the start of REXIN-G infusions, and 14 months after initial diagnosis.

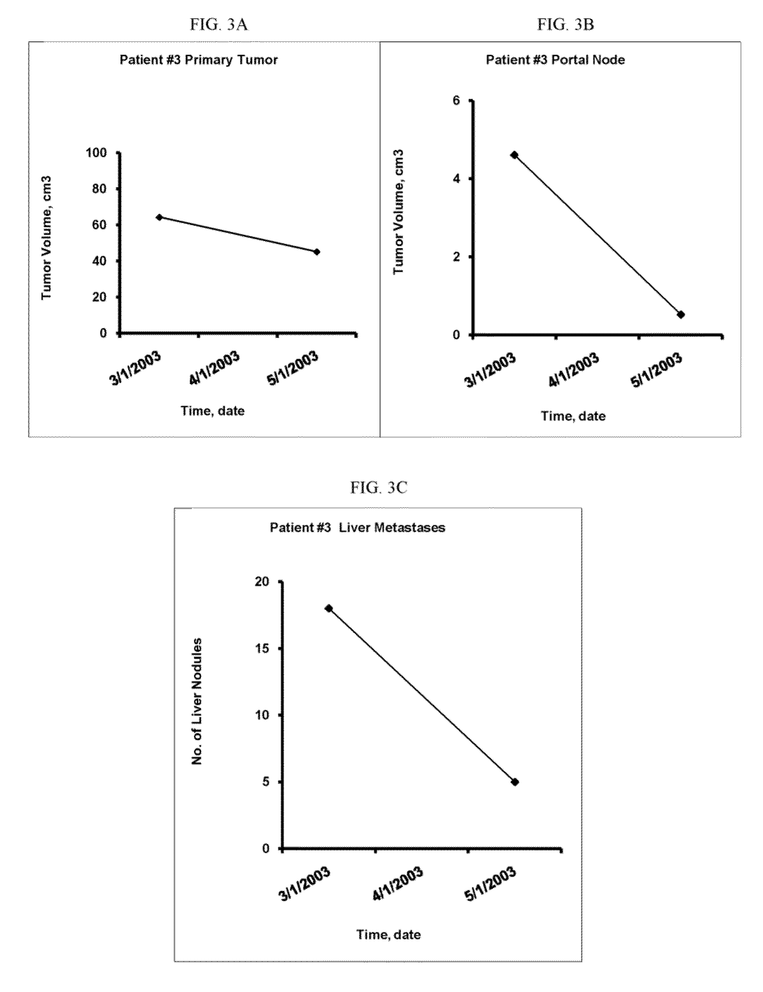

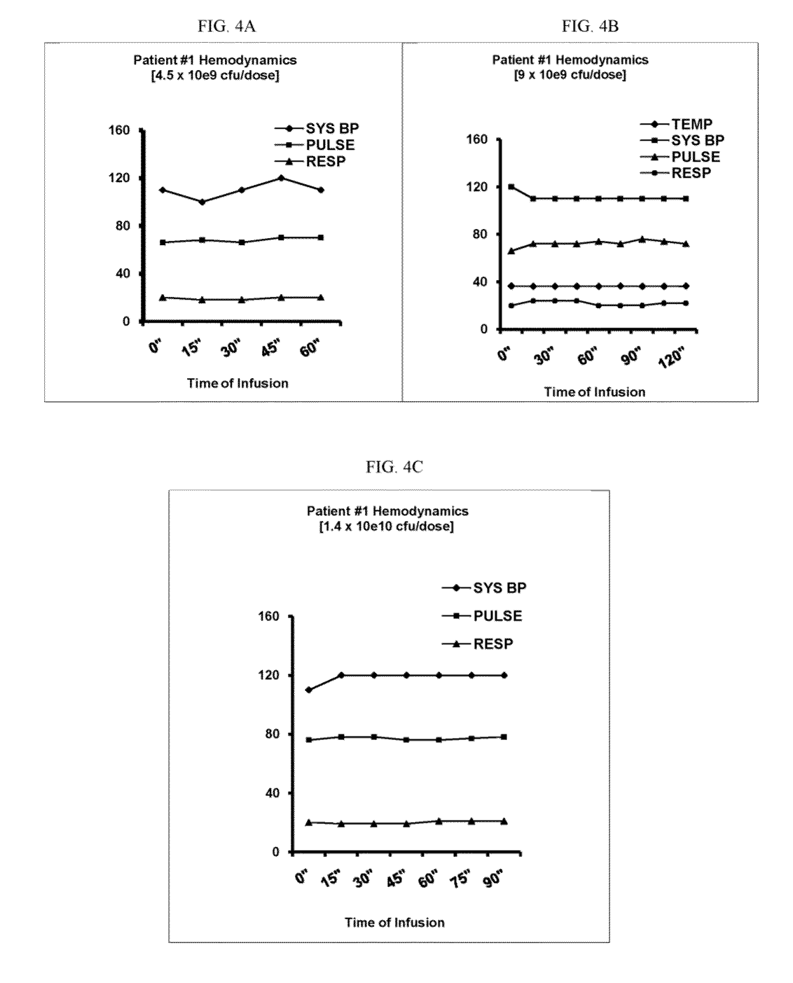

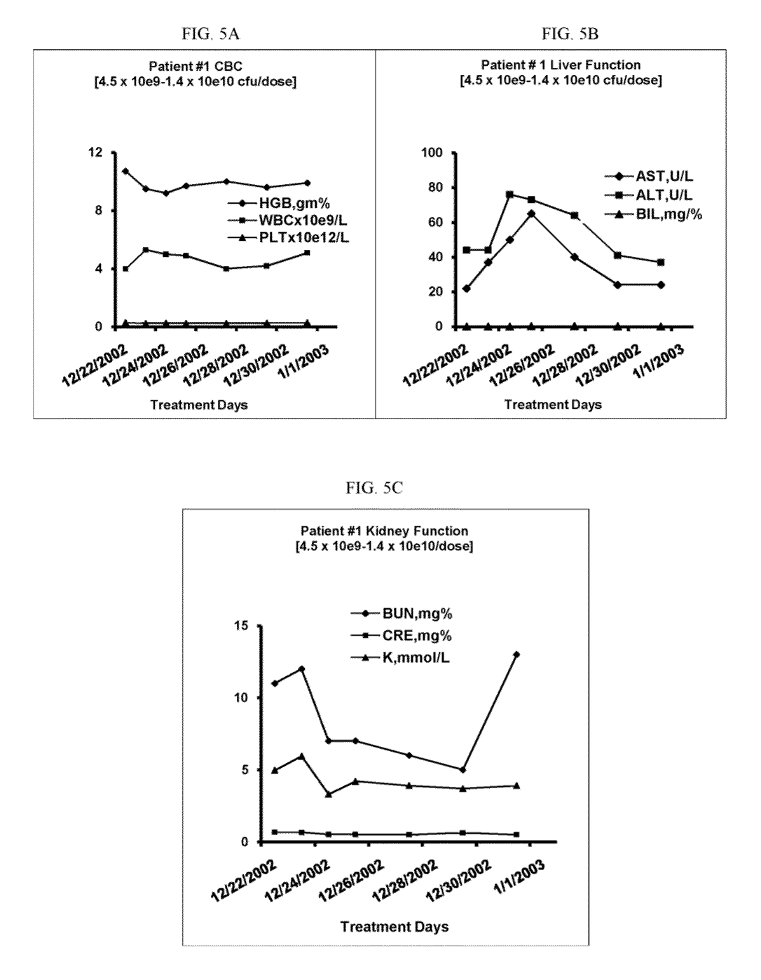

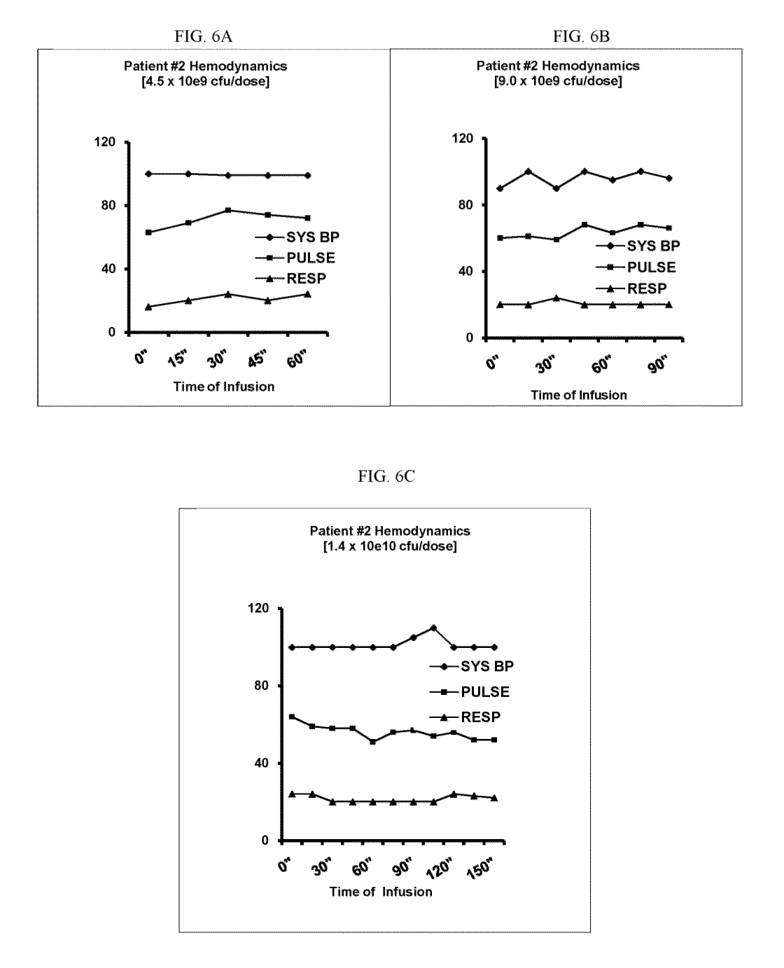

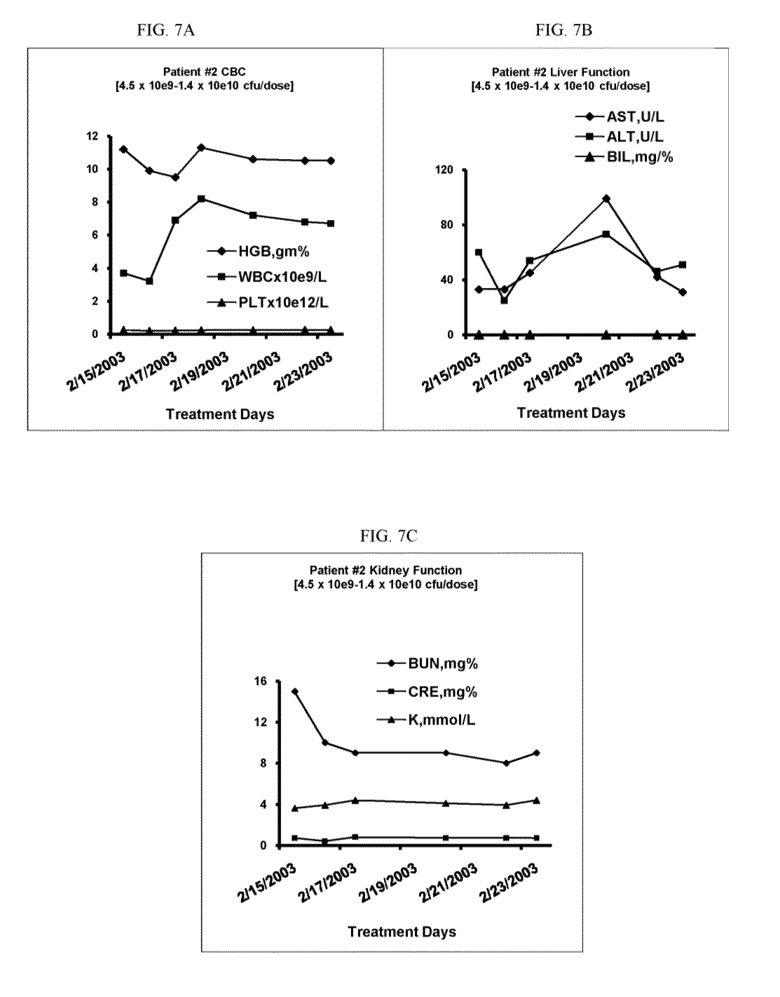

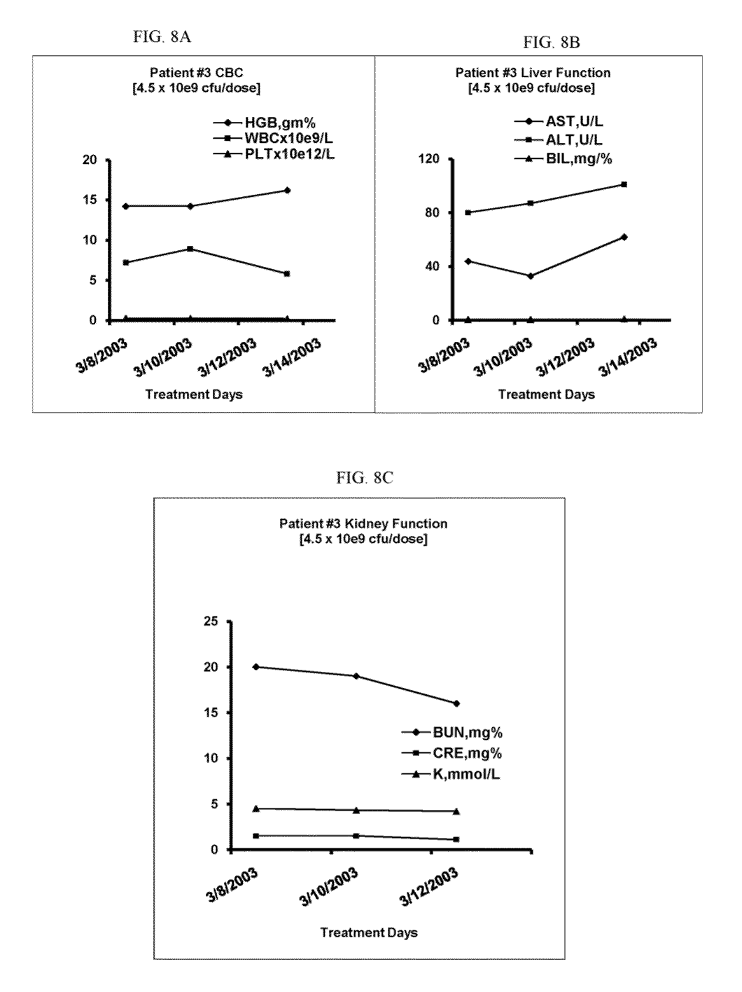

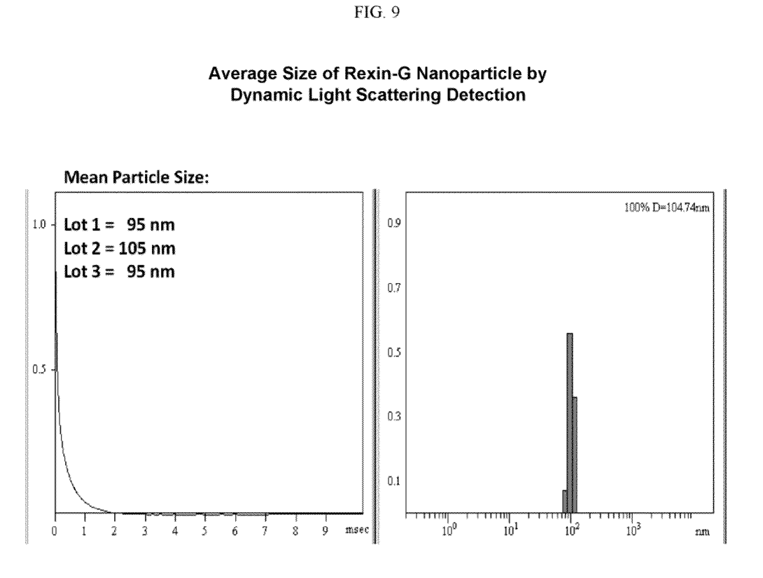

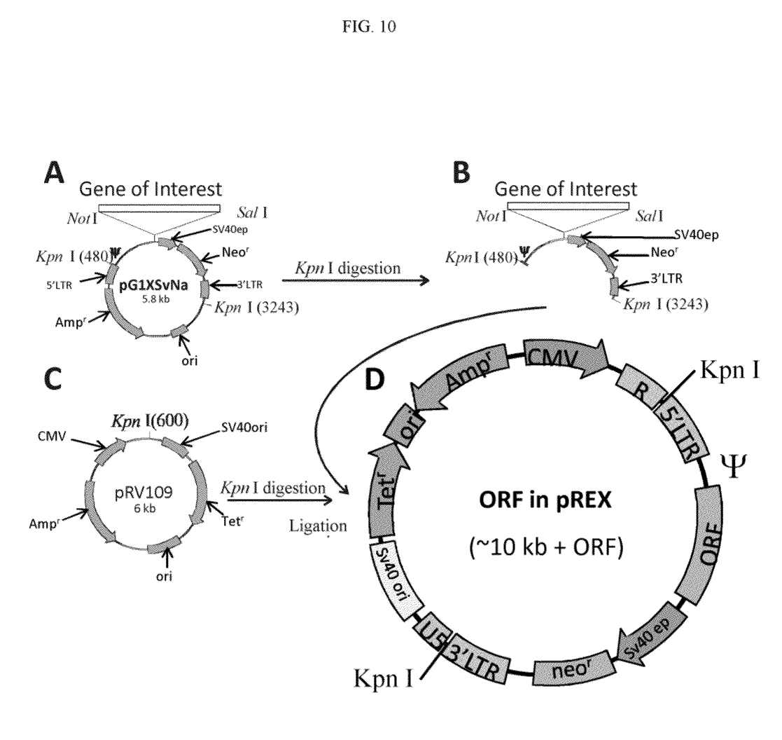

Patient #3, a 47 year old Chinese diabetic male was diagnosed to have Stage IVB adenocarcinoma of the body and tail of the pancreas, with numerous metastases to the liver and portal lymph node, confirmed by CT guided liver biopsy. Based on the rapid fatal outcome of Stage IVB adenocarcinoma of the pancreas, the patient was invited to participate in a second clinical protocol using REXIN-G frontline followed by gemcitabine weekly. A priming dose of REXIN-G was administered to sensitize the tumor to chemotherapy with gemcitabine for better cytocidal efficacy. The patient received daily IV infusions of REXIN-G at a dose of 4.5×10⁹ Units/dose for 6 days for a total cumulative dose of 2.7×10¹⁰ Units, followed by 8 weekly doses of gemcitabine (1000 mg/m²). On Day 62, follow-up abdominal CT scan showed that the primary tumor had decreased in size from 7.0×4.2 cm (Tumor Volume: 64.2 cm³) baseline measurement to 6.0×3.8 cm (Tumor Volume: 45 cm³) (FIG. 3A). Further, there was a dramatic reduction in the number of liver nodules from 18 nodules (baseline) to 5 nodules (FIG. 3C) with regression of the largest liver nodule from baseline 2.2×2 cm (Tumor Volume: 4.6 cm3) to 1×1 cm (Tumor Volume: 0.52 cm³) on Day 62 (FIG. 3B). By the RECIST criteria, Patient #3 is alive with stable disease on Day 133 follow-up, 4.7 months from the start of REXIN-G infusions and ˜5 months from the time of diagnosis.

Table 3 illustrates the comparative evaluation of over-all tumor responses in the three patients. Using the RECIST criteria, REXIN-G induced tumor growth stabilization in all three patients.

TABLE 3
Evaluation of Over-all Tumor Responses by RECIST

| | Patient No. | | |
|---|---|---|---|
| | 1 | 2 | 3 |
| Stage of | Recurrent IVB | IVA | IVB |
| Disease | | | |
| Previous Rx | Whipples | Ext. Beam | None |
| | Procedure | Radiation | |
| | Ext. Beam | 5 Fluorouracil | |
| | Radiation | Gemcitabine | |
| | Gemcitabine | | |
| Karnofsky | 0 | 0 | 0 |
| score before | | | |
| Treatment | | | |
| Treatment/s & | REXIN-G IV | REXIN-G IV | REXIN-G IV |
| Dose | (3.0 × 10e11 U) | (1.8 × 10e11 U) | (2.7 × 10e10 U) |
| | | | Gemcitabine IV |
| | | | [1000 mg/m² × 8] |
| Response | Tumor growth | Tumor growth | Tumor growth |
| | stabilization | stabilization | stabilization |
| Duration of | 3.4 months | >5.5 months | >4.7 months |
| Response | | | |
| Survival Status | Alive, with | Alive, with stable | Alive, with stable |
| | progressive | disease, | disease, 5 months |
| | disease, | 14 months from | from diagnosis |
| | 20 months from | diagnosis | |
| | diagnosis | | |

In this study, two methods were used to evaluate tumor responses to intravenous infusions of REXIN-G. Using the NCI-RECIST criteria that measures the sum of the longest diameters of target lesions that are greater than 2 cm, and the disappearance vs. persistence of all non-target lesions as points of comparison, 3 of 3 (100%) patients treated with REXIN-G had tumor growth stabilization for longer than 100 days (3 months) (Table 3). Evaluation of response by tumor volume measurement (formula: width²×length×0.52) (16), revealed that REXIN-G induced tumor regression in 3 of 3 (100%) patients, i.e., a 33-62% regression of metastatic lymphadenopathy in Patient #1 (Table 2), a 47% regression of the primary tumor in Patient #2 (FIG. 2C), and a 30% regression of the primary tumor, eradication of 72% (13/18) of metastatic liver foci, and an 89% regression of a metastatic portal node in Patient #3 as documented by imaging studies (MRI or CT scan) and caliper measurements (FIG. 3). Further, evaluation of safety showed that no dose-limiting toxicity occurred up to a cumulative vector dose of 3×10¹¹ Units, indicating that more vector may be given to achieve greater therapeutic efficacy. The REXIN-G vector infusions were not associated with nausea or vomiting, diarrhea, neuropathy, hair loss, hemodynamic instability, bone marrow suppression, liver or kidney damage.

### Example 6

### Clinical Trial A, Phase I/II, REXIN-G in Locally Advanced or Metastatic Pancreatic Cancer

Clinical Study A includes Phase I/II or single-use protocols investigating intravenous infusions of REXIN-G™ for locally advanced or metastatic pancreatic cancer following approval by the Philippine Bureau of Food and Drugs (BFAD) or by the United States Food Drug Administration (FDA), and the Institutional Review Board or Hospital Ethics Committee (Gordon et al. (2004) Int'l. J. Oncol. 24: 177-185). The objectives of the study were (1) to determine the safety/toxicity of daily intravenous infusions of REXIN-G™, and (2) to assess potential anti-tumor responses to intravenous infusions of REXIN-G™. The protocol was designed for patients with an estimated survival time of at least 3 months. After informed consent was obtained, six patients with locally advanced unresectable or metastatic pancreatic cancer were treated with repeated infusions of REXIN-G™. Five of the six patients had failed standard chemotherapy; these patients completed the intra-patient dose escalation protocol in Manila, Philippines and/or in Brooklyn, N.Y., USA, as follows: Days 1-2: 3.8×10e9 Units; Days 3-4: 7.5×10e9 Units; Days 5-6:1.1×10e10 Units; Days 7-10: 1.5×10e10 Units; Rest one week; Days 18-27: 1.5×10e10 Units. Two patients received 1 additional cycle, and one patient received 7 additional cycles. The sixth patient who presented with unresectable stage IV pancreatic cancer, received combination therapy as a first-line treatment, consisting of six days of IV REXIN-G (3.8×10e9 Units/day) followed by gemcitabine (1000 mg/m2) weekly for 8 weeks. For Clinical Study A, the REXIN-G preparation had a potency of 3×10e7 Units/ml.

Adverse events were graded according to the NIH Common Toxicity Criteria (CTCAE Version 2 or 3) (Common Toxicity Criteria Version 2.0. Cancer Therapy Evaluation Program. DCTD, NCI, NIH, DHHS, March, 1998.). To evaluate the clinical efficacy of REXIN-G™, we took into consideration the general cytocidal and anti-angiogenic activities of the agent (Gordon et al. (2000) Cancer Res. 60:3343-3347, Gordon et al. (2001) Hum. Gene Ther. 12: 193-204), as well as the dynamic sequestration of the pathotropic nanoparticles into metastatic lesions (Gordon et al. (2001) Hum. Gene Ther. 12: 193-204) that would affect the biodistribution or bioavailability of the targeted nanoparticles during the course of the treatment. Since the vector will accumulate more readily in certain cancerous lesions—depending on the degree of tumor invasiveness and angiogenesis—it is not expected to be distributed evenly to the rest of the tumor nodules, particularly in patients with large tumor burdens. This would predictably induce a mixed tumor response wherein some tumors may decrease in size while other tumor nodules may become bigger and/or new lesions may appear. Thereafter, with the normalization or decline of the overall tumor burden, the pathotropic surveillance function would distribute the circulating nanoparticles somewhat more uniformly. Additionally, the treated lesions may initially become larger in size due to the inflammatory reactions or cystic changes induced by the necrotic tumor. Therefore, two additional measures were used in the evaluation of objective tumor responses to REXIN-G treatment, aside from the standard Response Evaluation Criteria in Solid Tumors (RECIST; Therasse et al. (2000) J. Nat'l. Cancer Inst. 92:205-216): that is, (1) O'Reilly's formula for estimation of tumor volume: L×W²×0.52 (27 O'Reilly et al. (1997) Cell 88:277-285), and (2) the induction of necrosis or cystic changes in tumors during the treatment period. Thus, a decrease in the tumor volume of a target lesion of 30% or greater, or the induction of necrosis or cystic changes within the tumor were considered partial responses (PR) or positive effects of treatment. The one-sided exact test was used to determine the significance of differences between the PRs of patients treated with REXIN-G and historical controls with an expected 5% PR.

This initial Phase I/II study examines the safety and potential efficacy of an intra-patient dose escalation protocol. As shown in Table 4, partial responses (PR) of varying degrees were noted in 5 out of 6 patients treated with REXIN-G while stable disease was observed in the remaining patient. Three of 6 (50%) patients had a 30% or greater decrease in tumor size by RECIST or by tumor volume measurement, and 2 of 6 (33%) patients had necrosis of either the primary tumor or metastatic nodules by biopsy and/or by follow-up MRI/CAT scan. Further analysis of one particular patient (A3), in whom 6 of 8 liver tumor nodules disappeared by CT scan, was facilitated by means of a liver biopsy, which revealed an increased incidence of apoptosis, necrosis, and fibrosis within the tumor nodules similar to that observed in preclinical studies, along with the observation of numerous tumor infiltrating lymphocytes in the residual liver tumors of the biopsied liver. The presence of immunoreactive T and B lymphocytes infiltrating the residual liver tumors indicates that REXIN-G does not suppress local immune responses. Progression-free survival was greater than 3 months in 4 of 6 (67%) patients. Median survival after REXIN-G™ treatment in chemotherapy-resistant patients was 10 months, and median survival after diagnosis was 25 months. In contrast, the reported median survival of patients with pancreatic cancer who received either gemcitabine or 5-FU (standard treatments) as a first-line drug was 5.65 and 4.41 months after diagnosis, respectively (Burris et al. (1997) J. Clin. Oncol. 15:2403-2413). Using the one-sided exact test, the significance level of partial responses in REXIN-G-treated patients was <0.025 when compared to the PR rates of historical controls. These initial findings, albeit documented in a relatively small number of patients, are sufficient to indicate that REXIN-G is clinically effective, even in modest doses, is clearly superior to no medical treatment, and may be superior to gemcitabine when used as a single agent for the treatment of patients with advanced or metastatic pancreatic cancer.

TABLE 4
Objective Tumor Response, Progression-free Survival, and Overall Survival of Participants in
Clinical Study A

| | | | Status/Survival | Overall |
|---|---|---|---|---|
| Patient's Initials | | Progression | After REXIN- | Survival |
| Age | Objective Tumor Response | Free Survival | G Treatment | from Dx |
| A1 | Partial Response: Necrosis | 3.5 months | Expired | 23 months |
| 46 years | of primary tumor with 24% | | 10 months | |
| | decrease in tumor size; 33-62% | | | |
| | decrease in size | | | |
| | supraclavicular lymph nodes | | | |
| | Symptomatic relief of pain | | | |
| A2 | Partial Response (RECIST): | 9 months | Expired | 25 months |
| 55 years | 47% decrease in primary | | 13 months | |
| | tumor volume, followed by | | | |
| | complete disappearance of | | | |
| | the tumor | | | |
| | Symptomatic relief of pain | | | |
| A3 | Partial Response (RECIST): | 4 months | Expired | 19 months |
| 45 years | 47% decrease in primary | | 9 months | |
| | tumor volume; | | | |
| | disappearance of 6 of 8 liver | | | |
| | nodules; apoptosis and | | | |
| | necrosis of liver nodules in | | | |
| | biopsied liver | | | |
| | Symptomatic relief of pain | | | |
| A4 | Partial Response/Stable Ds: | 2 months | Expired | 48 months |
| 64 years | disappearance of 5 of 11 | | 8 months | |
| | liver nodules; stable primary | | | |
| A5 | Stable Disease: no change in | 2 months | Expired | 30 months |
| 53 years | primary tumor; one of 3 | | 10 months | |
| | liver nodules disappeared | | | |
| A6 | Partial Response (RECIST): | 5 months | Expired | 7 months |
| 46 years | 30% decrease in primary | | 7 months | |
| | tumor volume; | | | |
| | disappearance of 13 of 18 | | | |
| | liver nodules | | | |

All 6 patients tolerated the REXIN-G infusions well with no associated nausea or vomiting, diarrhea, mucositis, hair loss, or neuropathy. Three of six (50%) patients had symptomatic relief of pain. There was no significant alteration in hemodynamic function, bone marrow suppression, liver, kidney or any organ dysfunction that was related to the investigational agent. The only adverse events that were attributed as definitely related to the investigational agent were generalized rash and urticaria in 2 of 6 patients (Grade 1-2), and those attributed as possibly related were chills and fever in 2 of 6 patients (Grade I). The limited number of treatment-emergent adverse events observed in this study suggests that REXIN-G administered intravenously at these escalating doses is a relatively safe therapy.

### Example 7

### Clinical Study B, Phase I/II REXIN-G in Various Advanced or Metastatic Solid Tumors

Clinical Study B represents an expansion of Clinical Study A. Based on the encouraging results of the initial clinical experiences with REXIN-G, the Phase I/II study was expanded to further determine the safety and potential efficacy of a higher dose of REXIN-G, to extend the clinical indication to all advanced or metastatic solid tumors that are refractory to standard chemotherapy, and to adjust the treatment schedule and protocol to enable outpatient treatment. The objectives of this study were (1) to determine the safety/toxicity of daily intravenous infusions of REXIN-G, and (2) to assess potential anti-tumor responses to intravenous infusions of REXIN-G at a higher dose level. The protocol was designed for patients with an estimated survival time of at least 3 months. After informed consent was obtained, ten patients with metastatic cancer originating from either the ectoderm (melanoma, 1; squamous cell CA of larynx, 1), the mesoderm (leiomyosarcoma, 1) or the endoderm (pancreas, 2; breast, 2; uterus, 1; colon, 2), and one newly diagnosed previously untreated patient with metastatic pancreatic cancer who had refused chemotherapy (total number. of patients=11), received intravenous REXIN-G as a single agent at a dose of 3.0×10e10 Units per day for a total of 20 days, according to the following treatment schedule: Days 1-5, 8-12, 15-19, and 22-26; Monday to Friday with week-end rest period. An improved GMP manufacturing and bioprocessing protocol enabled the production of REXIN-G at substantially higher titers, such that the preparations used for Clinical Study B exhibited a vector potency of 7×10e8 Units/ml.

Adverse events were graded according to the NIH Common Toxicity Criteria (CTCAE Version 2 or 3) (Common Toxicity Criteria Version 2.0. Cancer Therapy Evaluation Program. DCTD, NCI, NIH, DHHS, March, 1998.). To evaluate the clinical efficacy of REXIN-G, we took into consideration the general cytocidal and anti-angiogenic activities of the agent (Gordon et al. (2000) Cancer Res. 60:3343-3347, Gordon et al. (2001) Hum. Gene Ther. 12: 193-204), as well as the dynamic sequestration of the pathotropic nanoparticles into metastatic lesions (Gordon et al. (2001) Hum. Gene Ther. 12: 193-204) that would affect the biodistribution or bioavailability of the targeted nanoparticles during the course of the treatment. Since the vector will accumulate more readily in certain cancerous lesions—depending on the degree of tumor invasiveness and angiogenesis—it is not expected to be distributed evenly to the rest of the tumor nodules, particularly in patients with large tumor burdens. This would predictably induce a mixed tumor response wherein some tumors may decrease in size while other tumor nodules may become bigger and/or new lesions may appear. Thereafter, with the normalization or decline of the overall tumor burden, the pathotropic surveillance function would distribute the circulating nanoparticles somewhat more uniformly. Additionally, the treated lesions may initially become larger in size due to the inflammatory reactions or cystic changes induced by the necrotic tumor. Therefore, two additional measures were used in the evaluation of objective tumor responses to REXIN-G treatment, aside from the standard Response Evaluation Criteria in Solid Tumors (RECIST; Therasse et al. (2000) J. Nat'l. Cancer Inst. 92:205-216): that is, (1) O'Reilly's formula for estimation of tumor volume: L×W²×0.52 (27 O'Reilly et al. (1997) Cell 88:277-285), and (2) the induction of necrosis or cystic changes in tumors during the treatment period. Thus, a decrease in the tumor volume of a target lesion of 30% or greater, or the induction of necrosis or cystic changes within the tumor were considered partial responses (PR) or positive effects of treatment.

This study extends the initial Phase I/II pancreatic cancer protocols with dose intensification and expanded clinical application to all solid tumors. As shown in Table 5, partial responses of varying degrees of either the primary tumor or the metastatic nodules were noted in 7 of 11 (64%) patients. Five of 11 (45%) patients developed necrosis and apoptosis of the primary tumors and/or metastatic nodules by either biopsy or CT scan, and 5 of 11 (45%) patients had greater than 30% reduction in the size of the primary tumor or metastatic nodules by RECIST or tumor volume measurement. Two of 11 patients had stable disease, one patient with massive tumor burden had a mixed tumor response and one patient with a large tumor burden (˜50 liver nodules) had progressive disease.

TABLE 5
Objective Tumor Response, Progression-free Survival, and Overall Survival of Participants
in Clinical Study B

| | | | | Overall |
|---|---|---|---|---|
| Patient's Initials, | Over-all Tumor Response | Progression | Status/Survival | Survival |
| Age, Dx and Date | [Symptomatic Relief, Caliper, | Free | After REXIN- | from |
| of Dx | CT scan and MRI] | Survival | G Treatment | Diagnosis |
| B1 | Partial Response (RECIST): | 3 months | Alive | >6.6 years |
| 53 years | Apoptosis and necrosis of | | >13 months | |
| Breast Cancer | tumor nodule by biopsy; 50% | | | |
| | decrease in supraclavicular | | | |
| | node by PET/CT scan; | | | |
| B2 | Partial Response: Necrosis of | 3 months | Expired | 2 years |
| 58 years | supraclavicular lymph nodes | | 4 months | 4 months |
| Uterine Cancer | by CT scan; 33% decrease in | | | |
| | cervical lymph node by | | | |
| | calipers | | | |
| | Symptomatic relief from | | | |
| | nerve pain | | | |
| B3 | Stable Disease: no interval | 2 months | Alive | >3 years |
| 52 years | change in pulmonary nodules | | >7 months | 5 months |
| Breast Cancer | Symptomatic relief from | | | |
| | coughing and bone pain | | | |
| B4 | Partial Response: Necrosis | 3 months | Alive | >15 months |
| 41 years | and apoptosis of biopsied | | >6 months | |
| Melanoma | tumor nodules; 50% decrease | | | |
| | in tumor volume by CT scan | | | |
| B5 | Progressive Disease | N.A. | Alive | >11 months |
| 53 years | Symptomatic relief from pain | | >6 months | |
| Pancreatic Cancer | | | | |
| B6 | Partial Response (RECIST): | 3 months | Alive | >24 months |
| 48 years | 300% increase in upper | | >6 months | |
| Squamous Cell CA, | airway diameter; stable lung | | | |
| larynx | nodules | | | |
| | Regained voice | | | |

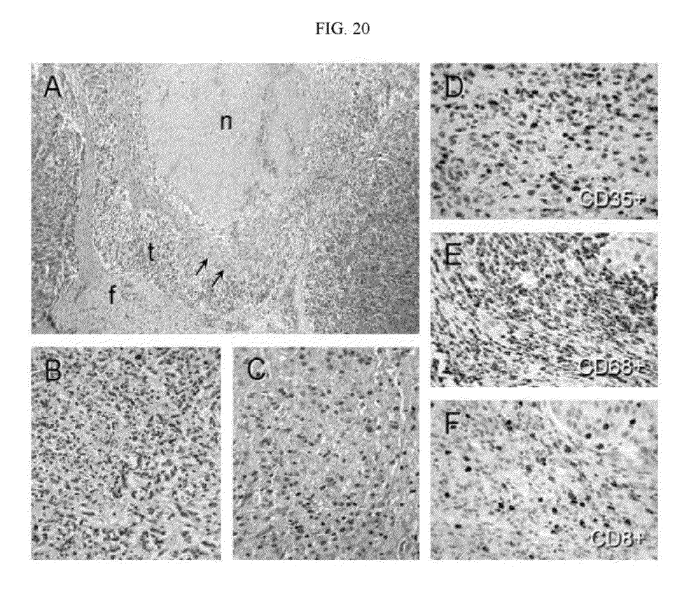

Progressive reduction of cancerous lymph nodes with repeated infusions of REXIN-G was consistently observed in patients with pancreatic cancer, and again in patients with uterine cancer, colon cancer, breast cancer and malignant melanoma, which is remarkable and meaningful in terms of understanding the pertinent pharmacodynamics. While it is well known that sentinel lymph node(s)—the first lymph node(s) to which cancer is likely to spread from a primary tumor—are of considerable importance to our understanding of the pathogenesis, diagnosis, and prospective treatment of metastatic disease, the conspicuous penetrance of REXIN-G into both regional and distant lymph nodes is both striking and auspicious (Tables 4 and 5). The clinical significance of the finding that the pathotropic nanoparticles in REXIN-G retain their bioactivity as they circulate throughout the body, not only accumulating in primary and metastatic lesions but also draining into lymph nodes with therapeutic impact, cannot be overstated. As shown in FIG. 20, a surgical biopsy of a cancerous lymph node from the inguinal region of a patient with malignant melanoma showed substantial necrosis (20-A), large areas of overt apoptosis, (20-B), and zones wherein hemosiderin-laden macrophages (20-C) are evacuating tumor debris. Moreover, immunohistochemical staining revealed significant mononuclear infiltrations with CD35+ dendritic cells (20-D), CD68+ macrophages (20-E), CD8+ killer T cells (20-F), and CD4+ helper T cells (not shown). The realization that the gene delivery function (i.e., cytocidal activity) of pathotropic nanoparticles remains active as it penetrates metastatic disease within sentinel lymph nodes, and does not disrupt but appears to work in concert with the immune system, reaffirms the potentiality of future cancer vaccinations in situ, using this targeted gene delivery system bearing a cytokine gene.

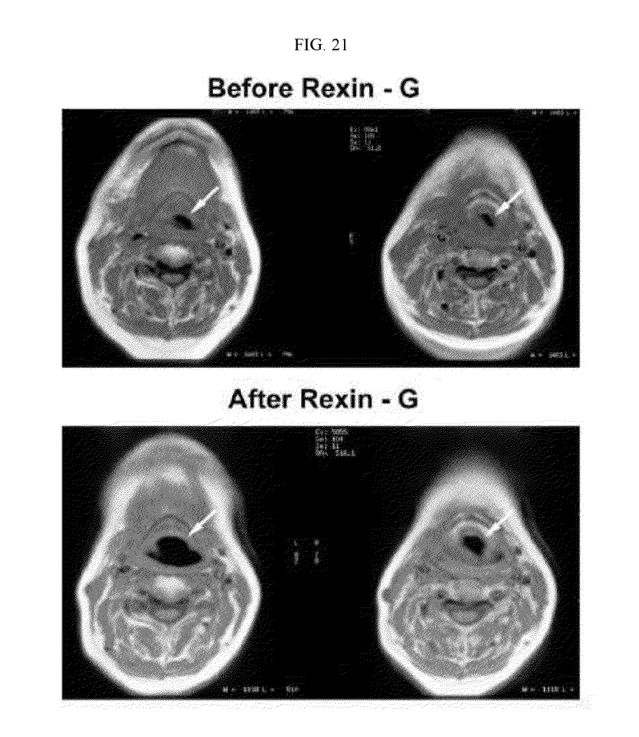

In another patient with squamous cell CA of larynx, a dramatic re-opening of the upper airway was documented by neck MRI (FIG. 21), which correlated with the patient's re-gaining of her voice. Progression-free survival ranged from one to greater than 5 months. Median survival time was greater than 6 months from the start of REXIN-G treatment, and greater than 24 months from diagnosis. Eight of 11 (72%) patients lived/are alive greater than 6 to 13 months after treatment with REXIN-G. Taken together, REXIN-G appears to have single agent activity in a broad spectrum of resistant tumor types. Further, it was noted that sustained therapeutic benefit was observed in the majority of the patients despite the brevity of the treatment.

All eleven patients tolerated the vector infusions well with no associated nausea or vomiting, diarrhea, mucositis, hair loss or neuropathy. Eight of 11 (73%) had symptomatic relief of pain, bloating, throbbing, hoarseness, and fatigue. There was no significant alteration in hemodynamic function, bone marrow suppression, liver, kidney or any organ dysfunction that was related to the investigational agent. The absence of treatment-related adverse events further suggests that, even in increased vector doses, REXIN-G is a relatively safe therapy. At this point, the absence of dose limiting toxicity, combined with compelling indications of single agent efficacy in a variety of different tumor types and the recent availability of higher potency formulations of REXIN-G encouraged the advancement and regulatory approval of clinical trials designed to focus on increased clinical efficacy and the optimization of treatment protocols.

### Example 8

### Clinical Study C, Expanded Access of REXIN-G in Metastatic Pancreatic and Colon Cancer and “the Calculus of Parity”

Clinical Study C involves a small group of patients who participated in an Expanded Access Program for REXIN-G for all solid tumors, a provisional program which was recently approved by the Philippine BFAD. The innovative protocol was designed to address (i.e., to reduce or eradicate) a given patient's total tumor burden as quickly, yet, as safely possible in order to prevent or forestall “catch up” tumor growth, and thereby minimize this confounding parameter. The estimated total dosage to be utilized was determined by an empiric calculation, referred to herein as “The Calculus of Parity” (referring to as a method of equality, as in amount, or functional equivalence). The basic formula takes into consideration the overall tumor burden, estimated from imaging studies (1 cm=approximately 1×10e9 cancer cells), an empiric performance coefficient (φ) or Physiological Multiplicity of Infection (P-MOI, in the terms of virology) for the targeted vector system (the P-MOI for a non-targeted vector system is essentially infinite), and the potency of the clinical-grade formulation (in Units/ml). Tumor burden was measured as the sum of the longest diameters of the tumor nodules, in centimeters, multiplied by 1×10e9 and expressed as the total number of cancer cells. An “operationally defined” performance coefficient (φ) or Physiological MOI (P-MOI) of 100 for REXIN-G was based on quantitative demonstrations of enhanced transduction efficiency of the targeted gene therapeutic system documented in a wide variety of preclinical studies, and upon the dose-dependent performance of REXIN-G observed in the crucible of the initial clinical trials. Importantly, the generation of a high-potency REXIN-G product (˜1.0×10e9 Units/ml) enabled the administration of calculated optimal doses of REXIN-G to be delivered intravenously without the risk of volume overload.

Pioneering Studies: After completion of the first 20 days of REXIN-G infusions, two patients with metastatic pancreatic cancer and one patient with metastatic colon cancer opted (with additional informed consent) to continue to receive intravenous REXIN-G™ infusions up to a total dose of ˜2.5×10e12 cfu over 6 weeks (1 patient) and 16 weeks (2 patients), respectively. This provided a Calculus of Parity which roughly paralleled the patients' estimated tumor burden based on CT scan or MRI.

Adverse events were graded according to the NIH Common Toxicity Criteria (CTCAE Version 2 or 3) (Common Toxicity Criteria Version 2.0. Cancer Therapy Evaluation Program. DCTD, NCI, NIH, DHHS, March, 1998.). To evaluate the clinical efficacy of REXIN-G, we took into consideration the general cytocidal and anti-angiogenic activities of the agent (Gordon et al. (2000) Cancer Res. 60:3343-3347, Gordon et al. (2001) Hum. Gene Ther. 12: 193-204), as well as the dynamic sequestration of the pathotropic nanoparticles into metastatic lesions (Gordon et al. (2001) Hum. Gene Ther. 12: 193-204) that would affect the biodistribution or bioavailability of the targeted nanoparticles during the course of the treatment. Since the vector will accumulate more readily in certain cancerous lesions—depending on the degree of tumor invasiveness and angiogenesis—it is not expected to be distributed evenly to the rest of the tumor nodules, particularly in patients with large tumor burdens. This would predictably induce a mixed tumor response wherein some tumors may decrease in size while other tumor nodules may become bigger and/or new lesions may appear. Thereafter, with the normalization or decline of the overall tumor burden, the pathotropic surveillance function would distribute the circulating nanoparticles somewhat more uniformly. Additionally, the treated lesions may initially become larger in size due to the inflammatory reactions or cystic changes induced by the necrotic tumor. Therefore, two additional measures were used in the evaluation of objective tumor responses to REXIN-G treatment, aside from the standard Response Evaluation Criteria in Solid Tumors (RECIST; Therasse et al. (2000) J. Nat'l. Cancer Inst. 92:205-216): that is, (1) O'Reilly's formula for estimation of tumor volume: L×W²×0.52 (27 O'Reilly et al. (1997) Cell 88:277-285), and (2) the induction of necrosis or cystic changes in tumors during the treatment period. Thus, a decrease in the tumor volume of a target lesion of 30% or greater, or the induction of necrosis or cystic changes within the tumor were considered partial responses (PR) or positive effects of treatment.

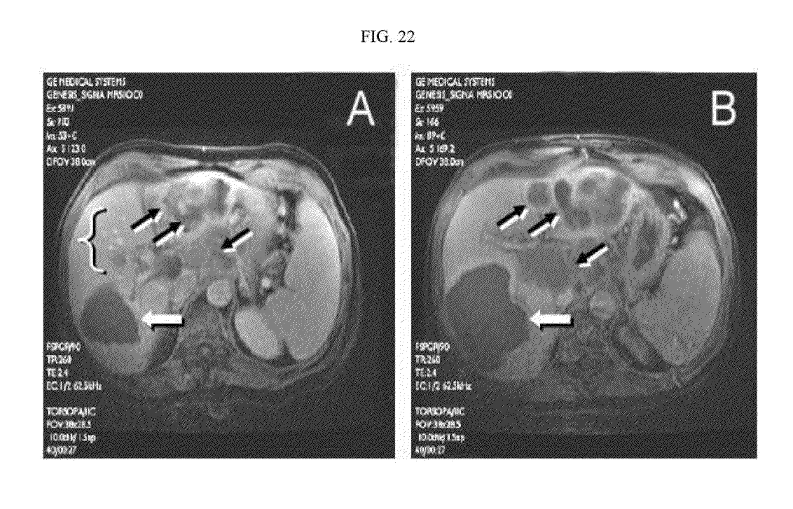

This study represents the initial report of clinical experience in an Expanded Access Program for REXIN-G for treating all solid tumors, introducing an innovative personalized dose-dense regimen referred to as the Calculus of Parity. In this preliminary yet important interim analysis, dramatic responses were noted in all three patients, each with an extensive tumor burden. In one patient (C1), the Calculus of Parity (or functional equivalence) approximated a cumulative dosage that led to liquefaction necrosis and cystic conversion of the unresectable pancreatic tumor and either cystic conversion or disappearance of all metastatic liver nodules on follow-up MRI (FIG. 22). Aspiration of one cystic tumor nodule was negative for malignant cells. In the second patient (C2), suffering from Stage IV colon cancer, a cumulative dosage approaching the predetermined Calculus of Parity was effective in reducing the bulk of the metastatic disease: 84% necrosis observed in the liver tumor nodules was documented by image analysis. In the third patient (C3), a significant decrease in the primary pancreatic tumor and in the number (from 28 to 12 lung nodules) and the size of pulmonary nodules were noted by CT scan. Progression-free survival and overall survival was greater than 6 months after REXIN-G treatment in two patients. These findings provide preliminary evidence to support the hypothesis that the Calculus of Parity may be used to determine the total cumulative dose of REXIN-G that would be needed to address a given patient's tumor burden, and thereby comprise an optimal induction regimen.

All three patients tolerated the vector infusions well with no associated nausea or vomiting, diarrhea, mucositis, hair loss or neuropathy. There were no acute alterations in hemodynamic function, bone marrow suppression, liver, kidney or any organ dysfunction that was related to the investigational agent. Two patients did develop anemia requiring red cell transfusion (grade 3), which was attributed as possibly related to subsequent bleeding into the necrotic tumors. One patient developed sporadic episodes of thrombocytopenia (grade 1-2) which was attributed as possibly related to the investigational agent. One patient died of acute fulminant staph epidermidis septicemia three months after REXIN-G treatment, which was NOT attributed to the investigational agent. The results of this patient's autopsy showed almost complete necrosis of the residual pancreatic tumor, and 75-95% necrosis of the metastatic tumors remaining in the liver and abdominal mesentery, with normal histology recorded in the bone marrow, heart, and brain. The lack of systemic toxicity associated with REXIN-G administration underscores the potential advantages of REXIN-G over standard chemotherapy in terms of efficacy in managing metastatic cancer, as well as other quality-of-life measures. In each case, the extent of the overall tumor destruction was impressive. The demonstration that a dose-dense regimen of REXIN-G, specifically tailored to overcome a patient's tumor burden, is capable of achieving these levels of efficacy underscores the need to further refine the Calculus of Parity, to define the optimal rate(s) of tumor eradication, and to discern the optimal supportive care for a patient undergoing post-tumoricidal wound healing.

The practice of the present invention will employ, unless otherwise indicated, conventional techniques of cell biology, cell culture, molecular biology, transgenic biology, microbiology, recombinant DNA, and immunology, which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor. N.Y., 1986).

### Example 9

### REXIN-G in a Metastatic Osteosarcoma Patient

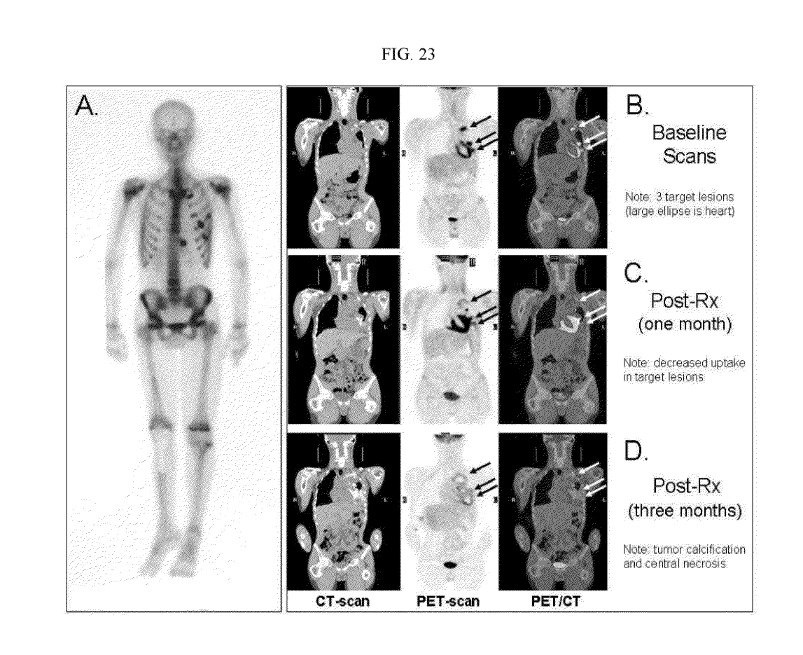

A 17-year-old white male, shown by radiography in FIG. 23A was diagnosed with osteosarcoma of the right tibia in December, 2003. He had received preoperative chemotherapy with cisplatin and adriamycin and high dose methotrexate followed by a limb salvage procedure. Post-operatively, he received courses of cisplatin and adriamycin (×2), and adriamycin and ifosfamide (×2), bringing the cumulative dose of adriamycin to 400 mg/m2. Chemotherapy was completed on February 2005. In March, 2006, a follow-up CT-scan showed two left-sided pulmonary metastases which were removed by VATS thorascopic surgery. From June to November, 2006, he received high dose methotrexate and ifosfamide, and then, underwent a thoracotomy in November, 2006. From December, 2006 to April, 2007, his lung tumors grew in size and number from a single lung nodule measuring 1 cm to over 10 lung and pleural-based nodules, with the largest lesion measuring 4.2 cm. This rapid rate of disease progression was compounded by the life-threatening location of the metastatic lesions, which involved both lungs, pericardium, and major vessels of the heart, with encroachment into the adrenal gland as well as the spine.

In April, 2007, the patient received REXIN-G on a compassionate basis. The patient was given 1×10e11 cfu REXIN-G intravenously twice a week for 4 weeks, followed by a 2-week rest period. A PET-CT scan obtained one week after completion of the first cycle showed a 28% increase in the sum of the target lesions, a 6% decrease in sum tumor density of target lesions, and a 33% reduction in the sum SUV max of 4 designated target lesions (see FIG. 23B vs. 23C). He continued to receive REXIN-G for an additional 4 weeks. A PET-CT scan obtained 2 weeks after completion of the 2^{nd} therapeutic course (see FIG. 23D) showed no new lesions, a 48% reduction in the sum SUV max of the 4 major target lesions, and a 539% increase in sum tumor density, indicating general calcification of the target lesions. Based, in part, on these positive tumor responses, the FDA approved a Phase II efficacy study of REXIN-G for metastatic osteosarcoma that is refractory to known therapies. In quantifying the objective tumor responses, a more comprehensive analysis of tumor response criteria was conducted—including PET criteria (metabolic activity), and CHOI criteria (tumor density), as well as RECIST (size only)—due to the tendency for osteosarcoma lesions to calcify rather than shrink with the cessation of tumor cell proliferation.

### Example 10

### REXIN-G in an Intractable Metastatic Osteosarcoma Patient

38 year-old black female with intractable metastatic osteosarcoma presenting with chemo-resistant osteosarcoma with tumor metastasis to the lungs. REXIN-G was used as a stand-alone therapy; 1-2×10e11 cfu, given 3× a week. Objective responses include attenuation of tumor metabolic activity, determined by PET criteria, sufficed to justify surgical resection. The approved dose escalation enables tumor control and a subsequent surgical remission; adjuvant REXIN-G therapy sustains remission for >2 years.

This Phase II efficacy study of REXIN-G for the treatment of chemo-resistant osteosarcoma brought forth an opportunity for the demonstrated anticancer activity of REXIN-G to serve as neoadjuvant therapy, thus setting the stage for a potentially curative surgery. In this case, a 38 year-old female was diagnosed in September, 1995 to have localized osteosarcoma of the left fibula. She underwent a limb salvage procedure in January, 1996 where the neoadjuvant/adjuvant therapy consisted of methotrexate, ifosfamide, cisplatin and adriamycin. Over the years, she developed multiple pulmonary metastases, requiring surgical resection of lung tumors, followed by re-institution of methotrexate, ifosfamide, adriamycin and cisplatin, plus interferon. In January 2008, she presented with chemo-resistant lung metastasis and was enrolled in the Phase II study using REXIN-G for osteosarcoma—which consisted of REXIN-G i.v. at a dose of 1-2×10e11 cfu, administered 3 times a week for 4 weeks, with a 2-week rest period.

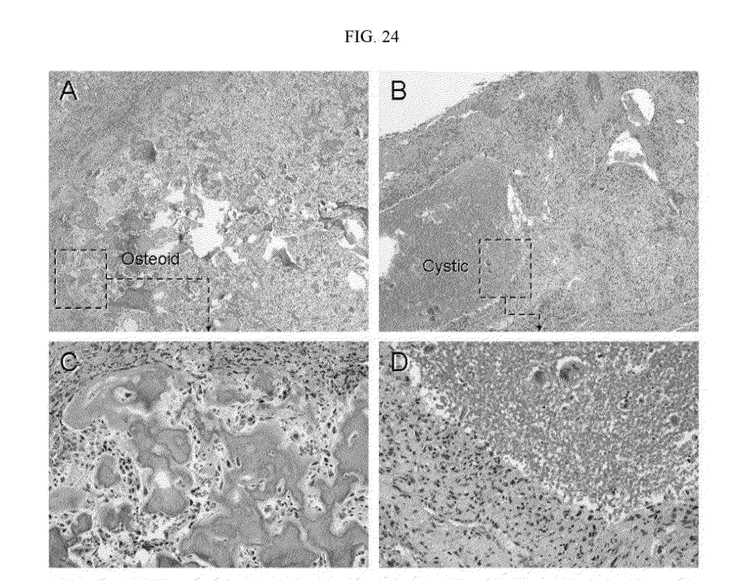

Having failed a number of aggressive chemotherapeutic regimens, and following previous rounds of surgical excisions, the cancer had recurred, presenting as a single lung metastasis. Repeated intravenous infusions of tumor-targeted REXIN-G included an intra-patient dose escalation in this case, which was approved across-the-board by the U.S. FDA, once adequate safety had been determined in ongoing clinical trials. Treatment with REXIN-G had a significant impact on the histology of the tumor, which upon surgical resection, was shown to have undergone cystic conversion of the one metastatic target lesion and ossification of an occult lesion, i.e., not seen by PET-CT scan (see FIG. 24). Thus, the patient received three treatment cycles followed by surgical resection of the residual lung tumors, and then 5 more cycles of REXIN-G post-operatively. To date, two years later, she enjoys a sustained remission with no evidence of disease.

### Example 11

### REXIN-G in an Intractable Ewing's Sarcoma Patient

36 year-old white male with intractable Ewing's sarcoma presented with chemotherapy-IGFR-therapy-resistant metastasis to the lung. Treatment protocol included REXIN-G as stand-alone therapy; 2×10e11 cfu infusions daily, 5× a week. Objective responses included attenuation of metabolic activity by PET; stabilization of tumor growth. Corroborative PET radiologic studies refine tumor response analysis

Ewing's sarcoma is a relatively rare malignancy of the bone and soft tissues, which is generally treated aggressively with multidrug chemotherapy, in addition to local disease control with surgery and/or radiation. In cases where progression to metastatic disease is apparent and the patient becomes refractory to standard therapies, the prognosis is exceedingly poor. In this case, a 36 year-old male was diagnosed with Ewing's sarcoma which was metastatic to lung and liver in July, 2004. H is multidrug chemotherapy regimens consisted of doxorubicin, dacarbazine, and ifosfamide, in addition to radiotherapy and surgical resection. After failing standard therapy, he was enrolled in a Phase I clinical study of a monoclonal antibody—the i.e., the RG1507 antibody by Hoffman-LaRoche—directed against the IGF receptor. The patient responded transiently to Insulin-like Growth Factor-1 Receptor (IGF-1R) therapy, which became ineffective over time. [Additional Note, in December of 2009, Roche/Genentech announced their decision to discontinue the clinical development of RG1507. Likewise, Pfizer suddenly suspended its Phase III figitumumab IGF-1R trial after a critical futility analysis].

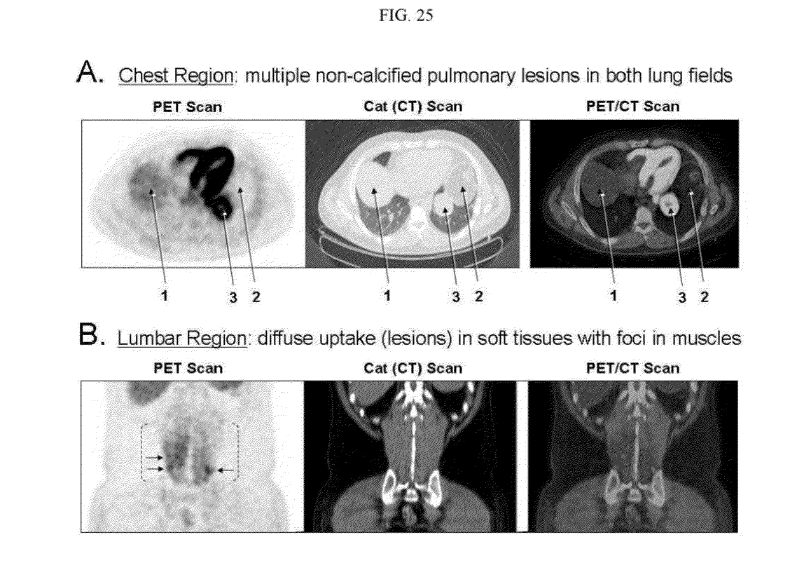

After failing this trial, the heavily pretreated patient received REXIN-G as stand-alone salvage therapy administered 5 days a week in an advanced Induction Regimen: REXIN-G i.v., given two times each day at a dose of 2×10e11 cfu per infusion. A subsequent PET/CT scan showed the persistence of large tumor masses in the lungs, yet there was a marked attenuation of metabolic activity in two of the largest lung nodules, as determined by an analysis of the composite of radiologic images. As seen in FIG. 25, the location and amount of the progressive metastatic disease in the lungs was considerable at this point of REXIN-G salvage therapy; however, the anti-tumor activity of the 2× daily REXIN-G infusions became more-evident upon careful analysis of the PET/CT scans. An overlay of the CT scan, which simply shows the size of the major pulmonary lesions, with the PET scan (PET/CT scan), which reveal the actual metabolic activity within these tumors, uncovered the true extent of the impact on tumor growth, as two of the three of the major target lesions showed significantly reduced metabolic activity (FIG. 25A), while the third, a metabolically active lesion, exhibited a discernibly necrotic center. Moreover, similar comparative scans of the spinal musculature of the lumbar region (see FIG. 25B) reveal troublesome evidence of tumor metastases by PET/CT that was not recorded by CT scan alone. These noteworthy observations indicate that the understanding gained by CT scans alone, is of a very meager kind, and suggest that a refinement of tumor response criteria to include evaluation of tumor metabolic activity be considered when it comes to precision targeted molecular therapies.

After three REXIN-G treatment cycles, the patient—by responding favorably to REXIN-G monotherapy—qualified for enrollment in the GeneVieve protocol, consisting of REXIN-G plus Reximmune-C (i.e., tumor-targeted GM-CSF vaccine (3) in an effort to prompt localized immune responses within the residual tumors, which might, in principle, lead to additional anti-tumor activity and long lasting anti-tumor immunity.

### Example 12

### REXIN-G in an Intractable Metastatic Breast Cancer Patient

74 year-old white female with intractable metastatic breast cancer presenting with chemotherapy and hormone-resistant cancer metastases to lymph nodes and chest wall. REXIN-G was used as a stand-alone therapy; 2×10e11 cfu given 3× a week. Objective responses included tumor shrinkage enabling surgical resection of a residual tumor nodule. Tumor histology confirms more significant cytological efficacy, including favorable immune responses; survival >3-years following treatment.

This case is a 74 year-old white female with recurrent ductal carcinoma of the breast, metastatic to axillary lymph nodes and tissues of the chest wall. She was diagnosed in September 2001 to have infiltrating ductal carcinoma of breast, T3N2 stage, for which she underwent a right mastectomy in September 2001, received doxorubicin and cyclophosphamide, radiation to the chest wall, followed by docetaxel, and then Tamoxifen which was initiated in October 2002. The breast cancer was determined to be ER positive, and questionable for HER-2/neu positivity. The patient remained on Tamoxifen until November, 2006, when she recurred in the chest wall, supraclavicular, axillary, and mediastinal lymph nodes, and possibly bone. She was entered in a clinical trial using Faslodex from Nov. 30, 2006 to Jan. 25, 2007. The patient responded initially, but there was residual therapy-resistant disease that was confirmed by repeat CT scans on Feb. 8, 2006.

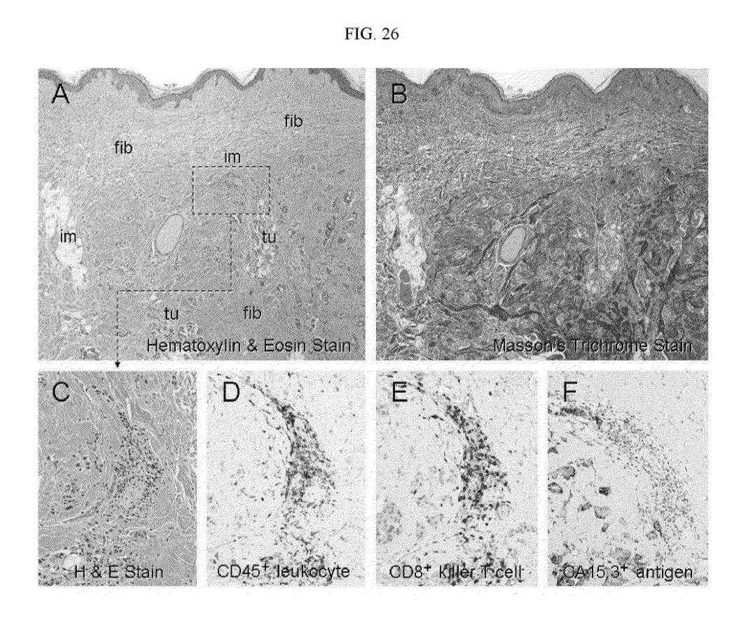

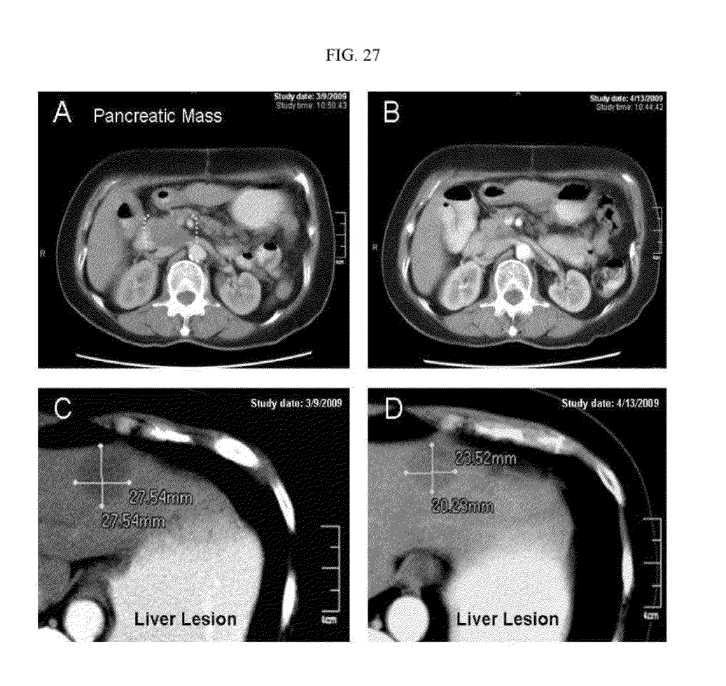

In this case of chemotherapy-resistant, hormone-resistant breast cancer, the recurrent disease was manifested in both in lymph nodes and the anterior chest wall. Repeated infusions of REXIN-G—1×10e1 cfu given three times a week for 3 weeks—resulted in regression of the chest wall tumor and axillary lymph nodes, enabling surgical resection of the solitary residual tumor. As shown in FIG. 26, the residual tumor was far from a flagrant proliferative tumor, appearing largely as a fibrotic mass (blue-staining material on Masson's trichrome stain) with scant but discernable apoptotic tumor cells accompanied by significant tumor infiltrating lymphocytes (TILs). Further characterization of the complement of TILs by specific immunocytochemical staining identified a significant proportion to be CD8+ killer T-cells, which are generally associated with a more favorable prognosis—a favorable prognosis that is affirmed by the continued survival of this patient, who is still alive more than three years after REXIN-G treatment.

### Example 13

### REXIN-G in an Intractable Metastatic Ovarian Cancer Patient

61 Year-old Asian female with intractable metastatic ovarian cancer presenting with chemotherapy-resistant cancer with metastasis to cerebrum and brain stem. REXIN-G was used as a stand-alone therapy; 2×10e11 cfu given 5 days a week. Objective responses included regression of metastatic brain lesions in frontal lobe and cerebellum. This is a first clinical demonstration of tumor control across the blood-brain barrier.

This 60 year-old patient was diagnosed to have adenocarcinoma of the left ovary, metastatic to omentum in May 2006. She underwent a total abdominal hysterectomy with bilateral salphingo-oophorectomy and received 6 cycles of paclitaxel and carboplatin with radiotherapy to the left pelvis. In November, 2009, she developed metastases to the left frontal lobe and right cerebellum, associated with severe depression and lethargy. She then received relatively intensive doses of REXIN-G monotherapy i.v. at 2×10e11 cfu per dose, given 5 days a week for 8 weeks. This intensive REXIN-G treatment resulted in substantial improvements in her depression and cognition, concomitant with regression of the cerebral and cerebellar metastatic foci.

This is not the first demonstration of REXIN-G single-agent efficacy seen in ovarian cancer, for objective tumor responses by RECIST have been recorded previously (data not shown). This case is particularly noteworthy as one of the first documented demonstrations of clinical efficacy—achieved by simple intravenous infusion—that reached across the blood-brain barrier. Whether the transport of these therapeutic doses of tumor-targeted REXIN-G nanoparticles across the blood-brain barrier and/or the choroid plexus is mediated by the retrovector surface envelope proteins or by some mechanism(s) of capillary permeability related to the disease histopathology, it is clear that REXIN-G exhibits sufficient penetrance and therapeutic mass action concentrated at the level of the individual brain tumors to cause the anatomical regression of these lesions.

### Example 14

### REXIN-G in a Metastatic Prostate Cancer Patient

91 year-old with metastatic prostate cancer presenting with primary tumor with extensive painful bone metastases. REXIN-G was used as a stand-alone therapy; 2×10e11 cfu, given 3× per week. Objective responses included eradication of the primary tumor and non-progression of bone metastases, resulting in progressive relief from bone pain and increased mobility. This is the first clinical demonstration of REXIN-G single-agent efficacy in advanced metastatic prostate cancer.

This 91 year-old male was diagnosed to have metastatic prostate cancer in April, 2009. He presented with a primary prostate gland malignancy with involvement of the urinary bladder floor, seminal vesicles, and obstructive uropathy, resulting in bilateral hydronephrosis; also evident was a high PSA level and extensive skeletal metastasis (skull, scapulae, sternum, vertebrae, ribs, pelvis, iliac wings, ischium, pubic bones, and femur) associated with debilitating bone pain to the extent that the patient was bedridden with ensuing decubitus ulcers. Due to the advanced age of this patient, first-line treatment with toxic chemotherapies and/or radiation therapy was precluded. Instead, the patient received REXIN-G i.v., 2×10e11 cfu per dose given three times a week for 8 weeks. Among the first distressing symptoms to abate was the severity of the bone pain followed by progressive relief from the sequelae of hydronephrosis. Follow-up abdominal sonogram, CT scans, and bone scans showed a normal prostate gland and kidneys, with non-progression of the bone metastases; in addition to subjective relief from pain, there was a significant reduction in serum PSA levels. The elderly patient was eventually able to walk again with the aid of a walker, to participate in daily activities, and to resume his employment.

### Example 15

### REXIN-G in an Intractable Metastatic Pancreatic Cancer Patient

54 year-old Asian female with intractable metastatic pancreas cancer presenting with chemo-resistant unresectable pancreas cancer metastatic to liver, abdominal lymph nodes, and lung. REXIN-G was given as a stand-alone therapy; 2×10e11 cfu, given 3× a week. Objective responses included resolution of primary tumor and regression of liver metastasis by CT scan. Resolution of primary tumor after only 4 weeks of REXIN-G treatment

This 60 year-old female was diagnosed in January, 2009 to have pancreatic adenocarcinoma with metastasis to the mesentery, liver, and lungs. The patient underwent a biliary bypass and was treated with standard chemotherapy, gemcitabine 1000 mg/m² for 4 weeks, which soon failed and resulted in progression of the disease. In April, 2009, she started treatment with REXIN-G i.v. at 2×10e11 cfu per dose, given three times a week for 4 weeks. Follow-up CT scan at the end of 4 weeks showed complete regression of the primary tumor and reduction in the size of the liver metastasis (target lesion). As seen in FIG. 27, there was a prompt and discernable change in tissue density (CHOI criteria), as well as tumor size (RECIST) following REXIN-G treatment. After one notable cycle of REXIN-G administered as second-line therapy, this favorably-responding patient was enrolled in the GeneVieve Protocol, consisting of REXIN-G plus REXIMMUNE-C (targeted GM-CSF) personalized vaccine therapy. She completed the 6-month treatment with REXIN-G+REXIMMUNE-C without event, with no new lesions and confirmed stable residual disease, and is undergoing treatment with REXIN-G as maintenance therapy for another 6 months.

### Example 16

### REXIN-G in an Intractable Metastatic Pancreatic Cancer Patient

73 year-old white female with intractable metastatic pancreas cancer presenting with chemo-resistant with metastasis to liver and abdominal lymph nodes. REXIN-G was used as a stand-alone therapy; 3×10e11 cfu, given 3× a week. Objective responses included complete clinical remission gained by maintaining treatment for 9 months. First demonstration of REXIN-G-induced clinical remission in a patient presenting with metastatic chemotherapy-resistant pancreatic cancer.

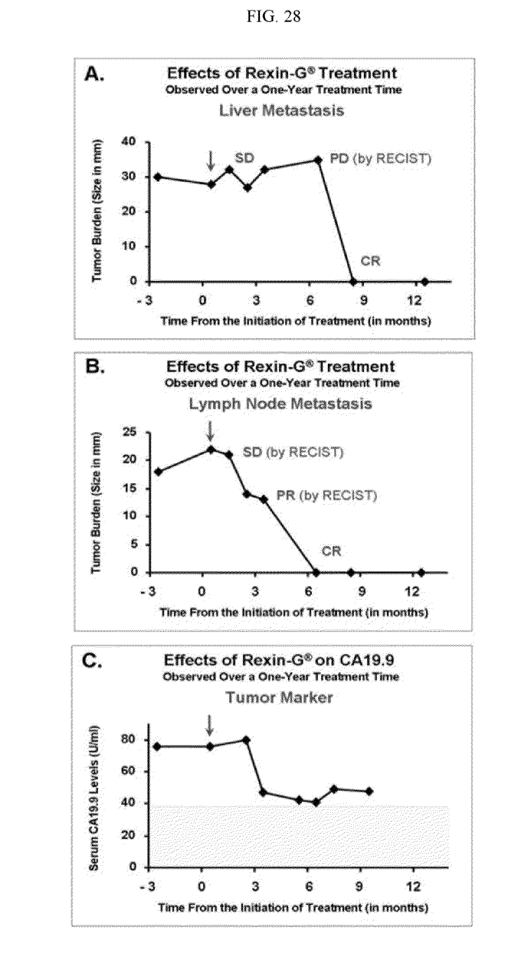

This 73 year-old female was diagnosed to have adenocarcinoma of pancreas in June, 2006. The patient underwent a Whipple's procedure in July, 2008 and received adjuvant therapy with 5-FU from September 2006 to October 2006, followed by gemcitabine from November, 2006 to February, 2007. She suffered tumor recurrence in the liver and abdominal lymph nodes in October, 2008, and was subsequently enrolled in a Phase I/II study of REXIN-G for gemcitabine-resistant pancreas cancer. She received REXIN-G i.v., at 3×10e11 cfu per infusion three times a week for 4 weeks followed by a 2-week rest period (comprising one treatment cycle). There were no new lesions during six months of REXIN-G treatment, indicating stable disease (SD, see FIG. 28A); however, there was some concern that one of the remaining liver lesions appeared to be slightly larger (by RECIST), which could be suggestive of progressive disease (PD). A further, more comprehensive analysis of objective tumor responses, including the progressive reduction in size of the target lymph node lesion (FIG. 28B) and a sustained drop in CA19.9 levels to near-normal levels (FIG. 28C) encouraged the Principal Investigator to hold-the-course of REXIN-G treatment—resulting, ultimately, in a complete clinical remission (CR, see FIG. 28A), as the remaining liver lesion was promptly resolved.

The observed absence of new lesions during repeated cycles of REXIN-G treatment, along with the achievement of stable disease (SD) represent significant clinical benefits, which should not be underestimated, in light of the predictable behavior of pancreatic cancer and the molecular mechanisms of action of REXIN-G. The continued treatment of this noteworthy pancreatic cancer patient, who was declared to be in clinical remission after 9 months of REXIN-G treatment, serves as a reminder that the eradication of metastatic liver lesions may occur promptly via apoptosis and anti-angiogenesis, or resolve gradually with the onset of fibrosis and tumor infiltrating lymphocytes (10), in which case it is of considerable benefit to continue to hold-the-course of REXIN-G treatment. This pancreas cancer patient enjoys a sustained remission for greater than 16 months from the initiation of REXIN-G treatment.

### Example 17

### REXIN-G in an Intractable Metastatic Pancreatic Cancer Patient

50 year-old white female with intractable metastatic pancreas cancer presenting with chemotherapy-resistant post Whipple's recurrence, metastasis to liver. REXIN-G used as a stand-alone therapy; 4×10e11 cfu given 3× a week. Objective responses included halting of tumor progression with disappearance of liver metastasis. A single residual tumor is excised after 6 months of REXIN-G therapy. Surgical remission is enabled by REXIN-G treatment, providing direct histological evidence of the molecular-mechanisms of action.

This 50 year-old female was diagnosed in August 2007 to have adenocarcinoma of the pancreas. The patient underwent a Whipple's procedure followed by a course of adjuvant chemotherapy consisting of gemcitabine and capecitabine from November 2007 to March 2008. In February, 2009, follow-up CT scan showed several foci of liver metastasis. She was then entered into a Phase I/II study of REXIN-G for gemcitabine-resistant pancreas cancer in March, 2009, where she received 4 cycles of REXIN-G i.v. at 4×10e11 cfu per dose administered three times a week, which resulted in the stabilization of disease progression, the prevention of new lesions and the eradication of one of two metastatic liver nodules (target lesions). The prevention of new lesions from occurring during the REXIN-G treatment period enabled the Principle Investigator to recommend a surgical resection of the one solitary residual tumor; which was promptly excised and embedded for histological examination.

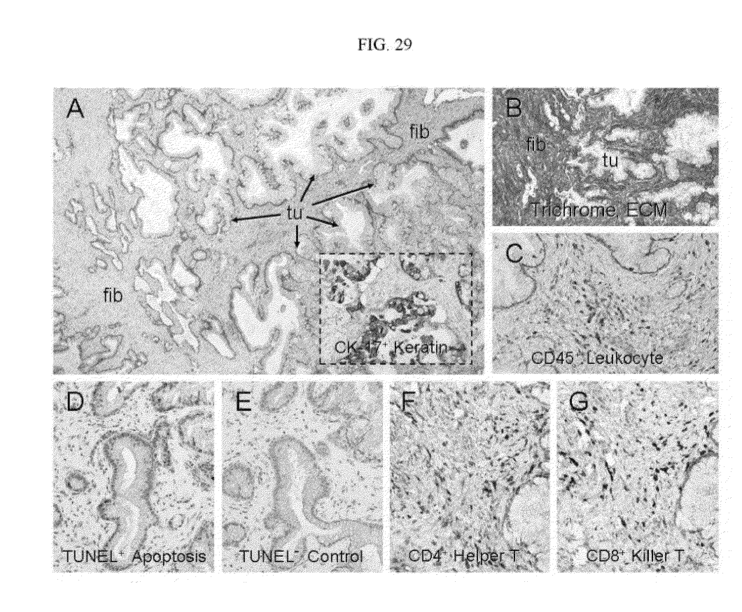

The timely treatment of this patient with REXIN-G—as neoadjuvant, immediately prior to the surgical procedure—enabled an opportunistic examination of REXIN-G in action within the metastatic lesion. As shown in FIG. 29A, a significant proportion of the volume of this REXIN-G pretreated tumor is composed of fibrosis and extracellular matrix proteins (29B), while the remainder of the residual tumor appears to be a rather slow growing and relatively pseudo-differentiated array of columnar/ductal structures in various stages of degeneration. This observation confirms the assertion that the objective response to treatment may be grossly underestimated by mere RECIST measurements. More-remarkably, REXIN-G appears to have induced massive amounts of apoptosis of the remaining cancer cells (see TUNEL Stain in FIG. 29D), as well as visible karyorrhexis—which is evident all along the borders of the pseudo-glandular structures. While the patient's local immune response is far from robust, with sporadic infiltration of CD45+ leukocytes observed within the lesion (29C), the cellular infiltrate consisted majorly of CD4+ helper T-cells (29F) and CD8+ killer T-cells (29G).

In addition to controlling the growth and spread of metastatic disease in Stage IV pancreatic cancer using REXIN-G as stand-alone therapy, this case is particularly noteworthy: for REXIN-G, by acting as an effective adjuvant therapy, enabled a definitive surgical remission from this deadly form of cancer—which is important for both clinical and surgical oncologists to consider. Post-operatively, the patient resumed REXIN-G treatment, after healing from the procedure, and continues to enjoy sustained clinical remission for >11 months after treatment initiation.

### Example 18

### REXIN-G in an Intractable Ewing's Sarcoma Patient

47 year-old white male with intractable metastatic pancreas cancer presenting with primary pancreatic mass with extensive liver and abdominal lymph node metastases. REXIN-G was used as a first-line treatment with gemcitabine; REXIN-G, 2-3×10e11 cfu, given 5 days a week; plus gemcitabine 1000 mg/m2, given weekly×7 weeks. Objective responses included prompt regression of primary tumor with 40% reduction in CA19.9 level. Demonstration of first-line combination therapy with REXIN-G plus Gemcitabine, devised to potentiate tumor responses to the oncolytic antimetabolite.

Presenting with symptoms of fever and jaundice, this 48 year-old white male was diagnosed to have adenocarcinoma of the pancreas with an extensive metastatic tumor burden involving the liver and abdominal lymph nodes in November of 2009. While a Whipple's procedure was precluded by the presence of the metastatic disease, a biliary bypass was performed with choledoco-duodenal anastomosis and cholecystectomy. Responding to an urgent request for compassionate use of REXIN-G as first-line therapy, and following all the qualifications and ramifications of international regulatory approvals, the patient was treated with a combination of REXIN-G—given i.v. at 2×10e11 cfu per dose, 5 days a week—plus gemcitabine administered at a weekly dose of 1000 mg/m² for a total seven weeks. Following the initial course of this first-line combination therapy, a follow-up MRI showed significant regression of the pancreatic mass and a general stabilization of the liver metastases and abdominal lymphadenopathy. These radiologic indications of tumor control were accompanied by a 30% reduction in the level of the tumor marker CA19.9, which is additionally noteworthy in light of studies suggesting that a timely decline in CA19.9 compares favorably with objective radiological responses as a strong indicator of time-to-progression, as well as overall survival, and may even serve as a surrogate endpoint (24, 25).

The gemcitabine was discontinued for a period of two weeks, due to a progressive elevation in liver enzyme levels (i.e., LFT elevation)—attributable to known gemcitabine toxicity in accordance with standard dose/treatment modification protocols; while the REXIN-G infusions were continued during this extended rest period. Notably, the liver function tests promptly normalized while the CA19.0 continued to fall to 40% of the initial values. With the relative safety of the combined therapy established, the dose of REXIN-G was raised to 3×10e11 cfu per dose administered three times per week during the next course of combined therapy. Presently, this patient is doing well and continuing on with additional rounds of REXIN-G/gemcitabine combined therapy in the hope that the limited oncolytic efficacy of the anti-metabolite may be enhanced by the targeted anti-angiogenic, anti-tumor activity of REXIN-G, which operates with a distinctly different molecular mechanism-of-action.

### Example 19

### REXIN-G Phase I/II and Phase I Clinical Trials

There are completed or active Phase I, I/II for pancreatic cancer, sarcoma, breast cancer, and Phase II studies of REXIN-G for osteosarcoma. Dose schedules are provided in Tables 6 and 7.

TABLE 6
Dosing schedules, no of patients, cumulative dose per cycle - USA

| | | | Dosing Schedule | Cumulative |
|---|---|---|---|---|
| Protocol | Title of | No. of | *Treatment not | Dose |
| No. | Protocol | Patients | repeated | given, cfu |
| C03-101 | Phase I, | 12 | Dose Level I: 7.5 × 10⁹ | 1.0 × 10¹¹ |
| No intra- | Pancreatic | | cfu qd × 7 days × 2 | |
| patient | CA | | weeks | |
| dose | | | Dose Level II: 1.1 × | 1.5 × 10¹¹ |
| escalation | | | 10¹⁰ cfu qd × 7 days × 2 | |
| | | | weeks | |
| | | | Dose Level III: 3 × 10¹⁰ | 6.0 × 10¹¹ |
| | | | cfu qd × 5 days × 4 | |
| | | | weeks | |

TABLE 7
Dosing schedules, no. of patients, cumulative dose per cycle - USA

| | | | Dosing Schedule | Cumulative |
|---|---|---|---|---|
| Protocol | Title of | No. of | *Treatment not | Dose |
| No. | Protocol | Patients | repeated | given, cfu. |
| C07-103 | Phase I/II, | 36 | Dose Level 0: 1 × | 8 × 10¹¹ |
| Intra- | Sarcoma | | 10¹¹ cfu TIW × 4 | |
| Patient | | | weeks | |
| dose | | | Dose Level I: 1 × | 12 × 10¹¹ |
| escalation | | | 10¹¹ cfu TIW × 4 | |
| | | | weeks | |
| | | | Dose Level II: 2 × | 24 × 10¹¹ |
| | | | 10¹¹ cfu TIW × 4 | |
| | | | weeks | |
| | | | Dose Level III: 3 × 10¹¹ | 36 × 10¹¹ |
| | | | cfu TIW × 4 weeks | |
| | | | Dose Level IV: 4 × 10¹¹ | 48 × 10¹¹ |
| | | | cfu TIW × 4 weeks | |
| C07-104 | Phase I/II, | 20 | Dose Level 0: 1 × 10¹¹ | 8 × 10¹¹ |
| Intra- | Breast CA | | cfu TIW × 4 weeks | |
| Patient | | | Dose Level I: 1 × 10¹¹ | 12 × 10¹¹ |
| dose | | | cfu TIW × 4 weeks | |
| escalation | | | Dose Level II: 2 × | 24 × 10¹¹ |
| | | | 10¹¹ cfu TIW × 4 | |
| | | | weeks | |
| | | | Dose Level III: 3 × 10¹¹ | 36 × 10¹¹ |
| | | | cfu TIW × 4 weeks | |
| | | | Dose Level IV: 4 × 10¹¹ | 48 × 10¹¹ |
| | | | cfu TIW × 4 weeks | |
| C07-105 | Phase I/II, | 20 | Dose Level 0: 1 × | 8 × 10¹¹ |
| Intra- | Pancreatic | | 10¹¹ cfu TIW × 4 | |
| Patient | CA | | weeks | |
| dose | | | Dose Level I: 1 × 10¹¹ | 12 × 10¹¹ |
| escalation | | | cfu TIW × 4 weeks | |
| | | | Dose Level II: 2 × 10¹¹ | 24 × 10¹¹ |
| | | | cfu TIW × 4 weeks | |
| | | | Dose Level III: 3 × 10¹¹ | 36 × 10¹¹ |
| | | | cfu TIW × 4 weeks | |
| | | | Dose Level IV: 4 × 10¹¹ | 48 × 10¹¹ |
| | | | cfu TIW × 4 weeks | |
| C07-110 | Phase II | 22 | Dose Level I: 1 × 10¹¹ | 12 × 10¹¹ |
| Intra- | Osteo- | | cfu TIW × 4 weeks | |
| patient | sarcoma | | Dose Level II: 2 × 10¹¹ | 24 × 10¹¹ |
| dose | | | cfu TIW × 4 weeks | |
| escalation | | | | |

Design and Methods—Objectives/Study Design/Endpoints: The primary objective of the Phase I/II study was determination of the clinical toxicity of escalating doses of REXIN-G as defined by patient performance status, toxicity assessment score, hematologic, and metabolic profiles. Secondary objectives included (i) evaluation of the potential of REXIN-G for evoking an immune response, recombination events and/or unwanted vector integration in non-target organs, and (ii) identification of an anti-tumor response to REXIN-G.

The study employed a modification of the standard Cohort design (Storer 1989). Each cohort of three could be expanded to six patients depending on toxicity or biologic activity. Maximum tolerated dose was defined as the highest safely tolerated dose, where ≦1 patient experienced dose-limiting toxicity (DLT), with the next higher dose level having at least two patients who experienced DLTs. DLT was defined as any grade 3, 4, or 5 adverse events considered possibly, probably, or definitely related to the study drug, excluding anticipated events such as grade 3 ANC lasting <72 hours, grade 3 alopecia, or any grade 3 or worse nausea, vomiting, or diarrhea (NCI Common Terminology Criteria for Adverse Events; CTCAE version. 3).

A Phase II efficacy component was incorporated in the on-going Phase I/II clinical trials by allowing additional treatment cycles to be given if the patient had <Grade I toxicity. Further, across the board dose escalations were allowed up to Dose Level II for patients with <Grade I toxicity when safety at the specified dose level was documented. The principal investigator was also allowed to recommend surgical resection/debulking and REXIN-G was continued if residual disease was found by histological examination or PET-CT scan.

Statistical Analysis (Phase I/II)—Primary evaluation of safety utilized information collected on all adverse events during the treatment period. Efficacy information was summarized for each dose as the number in each of the categories CR, PR, SD, and PD based on the RECIST, International PET and CHOI criteria. The number achieving any response (defined as CR, PR, SD and PD) was tabulated. In addition, information is reported for the following endpoints: tumor control rates (CR, PR or SD), progression-free survival and over-all survival. Progression-free survival and overall survival is summarized with Kaplan-Meier plots. Correlations among extent of tumor burden, tumor response, and dose level was also evaluated. Demographic and baseline information (e.g., extent of prior therapy) on study patients is tabulated. The following information is reported for adverse events observed in the study: dose level, type (organ affected or laboratory determination, such as absolute neutrophil count), severity and most extreme abnormal values for laboratory determinations) and relatedness to study treatment. For each dose, the number of patients experiencing any grade 3, 4, or 5 adverse event are reported, as well as the number of patients who experienced specific types of adverse events. Safety and some pharmacokinetic data, as well as anti-tumor activity/efficacy information are presented for accelerated approval of REXIN-G.

Phase I/II Sarcoma (Bone and Soft Tissue Sarcoma): 33 patients evaluable Table 8 shows the patient demographics for the Phase I/II sarcoma study (Chawla et al. 2009). The Sarcoma Study encompasses 14 types of sarcoma: osteosarcoma, Ewing's sarcoma, chondrosarcoma, liposarcoma, malignant fibrous histiocytoma, leiomyosarcoma, synovial cell sarcoma, fibrosarcoma, mixed malignant Mullerian tumor of ovary, malignant spindle cell sarcoma, angiosarcoma of heart, alveolar soft part sarcoma, rhabdomyosarcoma, and amelanotic schwannoma.

TABLE 8
Patient demographics (Phase I/II Sarcoma Study BB-IND# 11586)

| Categories | Total N (percent population) |
|---|---|
| Age, years | |
| Median | 48.8 |
| Range | (12.0-70.0) |
| Gender | |
| Female | 16 (44%) |
| Male | 20 (56%) |
| Race | |
| White | 31 (86.1%) |
| Black | 1 (2.8%) |
| Hispanic | 3 (8.3%) |
| Asian | 1 (2.8%) |
| Disease Stage | |
| Metastatic | 35 (97.2%) |
| Non-metastatic | 1 (2.8%) |
| Performance Score | |
| 1 | 36 (100%) |
| # Previous Chemotherapy | |
| Regimens | |
| Median | 4 |
| Range | 1-10 |

TABLE 9
Efficacy data on evaluable patients according to dose level (n = 33)

| | | | | Median | | |
|---|---|---|---|---|---|---|
| | Tumor | Tumor | Tumor | PFS by | Median | |
| Dose | Response | Response | Response | RECIST | OS | One-Year |
| Level (n) | by RECIST | by PET | by CHOI | (months) | (months) | Survival |
| 0 | 3SD, 3PD | 1PR, | 2PR, 4SD | 1.2 | 3.2 | 0% |
| (n = 6) | | 4SD, 1PD | | | | |
| I-II | 10SD, 4PD | 4PR, | 7PR, 7SD | 3.8 | 7.8 | 29% |
| (n = 14) | | 9SD, 1PD | | | | |
| III, IV | 9SD, 4PD | 3PR, | 1PR, | 4.1 | 12.2 | 40% |
| (n = 13) | | 8SD, 2PD | 10SD, 2PD | | | |

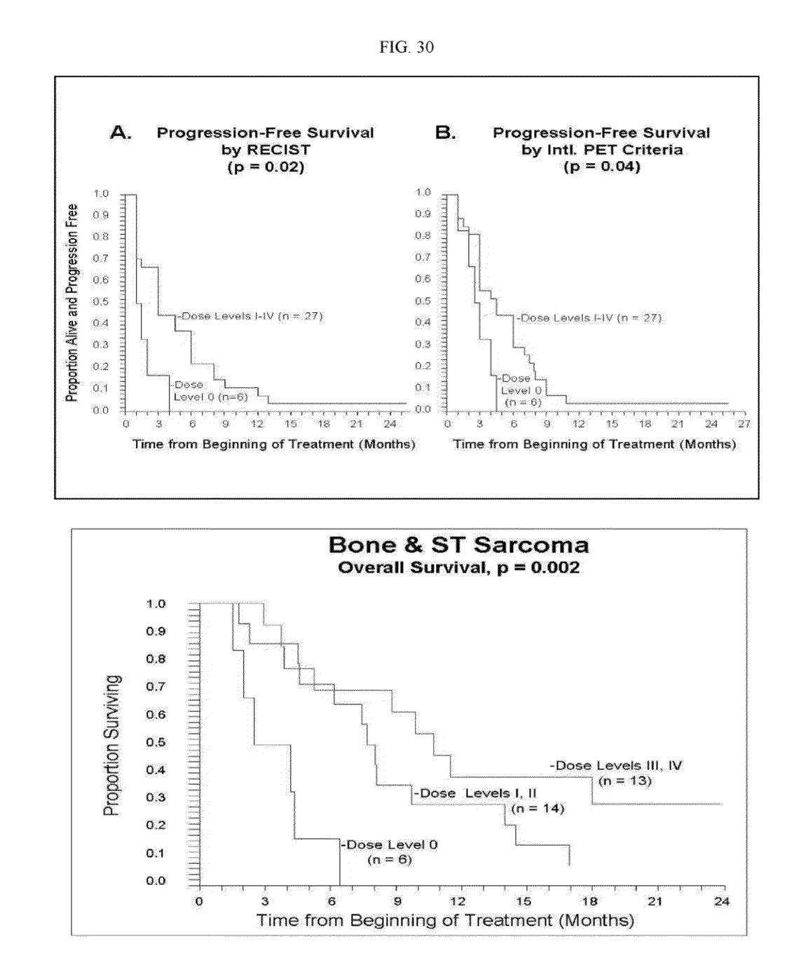

The International PET Criteria and CHOI criteria appear to be more sensitive indicators of early response to REXIN-G treatment. FIG. 30 shows a direct relationship between progression-free survival and REXIN-G dose. A significant dose-response relationship between progression-free survival and REXIN-G dosage was demonstrated at the 5% statistical level by the log rank test. The proportion of patients surviving is plotted on the vertical axis as a function of time from beginning of treatment, plotted on the horizontal axis. Evaluable patients are those patients who completed at least one treatment cycle and had a tumor response evaluation. FIG. 30C shows the Kaplan-Meier analysis of overall survival revealing a dose-response relationship between Overall Survival (OS) and REXIN-G dosage (n=33; p=0.002 in the treated groups). The significance in the Intention-to-Treat groups was p=0.016). The clinical data suggests that REXIN-G may exhibit significant anti-tumor activity, and may help control tumor growth, improve progression-free survival and overall survival in chemotherapy-resistant bone and soft tissue sarcoma.

Phase II Efficacy Studies of REXIN-G for Osteosarcoma: The goal of this study is to gain accelerated approval of REXIN-G as salvage therapy for osteosarcoma based upon the completion of a confirmatory single arm study in 20-30 patients with recurrent or metastatic osteosarcoma who are refractory to known therapies. The primary endpoint is clinical efficacy as measured by over-all response rates (either CR, PR or SD) by International PET criteria. The secondary endpoints are as follows: (1) clinical efficacy as measured by progression-free survival greater than one month and over-all survival of 6 months or longer, and (2) clinical toxicity as defined by patient performance status, toxicity assessment score, hematologic, and metabolic profiles, immune responses, vector integration in PBLs and recombination events.

Each treatment cycle will be six weeks: four weeks of treatment and two weeks of rest. Patients with ≦Grade I toxicity may have repeat cycles after the safety data and objective tumor responses are recorded. Initially, patients received REXIN-G i.v. at a designated dose level which was based on the estimated tumor burden as measured by PET-CT imaging studies. Subsequently, the protocol was amended to include an intra-patient dose escalation option if there was disease progression or a disease-related adverse event. Continued REXIN-G treatment enables confirmation of the beneficial anti-tumor effects of cumulative doses of REXIN-G in terms of disease stabilization and extension of over-all survival, as well as confirmation of the absence of cumulative toxicity, both of which were clearly demonstrated in a Phase I/II study of REXIN-G in metastatic bone and soft tissue sarcoma that had failed standard chemotherapy.

The principal investigator may recommend surgical debulking or resection after one or more treatment cycle/s, enabling the histologic characterization of treated tumors and comparison with known features of REXIN-G-treated tumors, which have been demonstrated in previous preclinical and clinical studies. These features include the presence of apoptotic tumor cells and endothelial cells (the primary mechanism of action of REXIN-G), and varying degrees of central necrosis with reactive inflammatory reaction, focal microhemorrhages (anti-angiogenic effects of REXIN-G resulting from the selective destruction of proliferative tumor endothelial cells), reparative fibrosis, and a characteristic complement of tumor infiltrating lymphocytes.

Post-operatively, repeat cycles may be given if residual disease is present either by histopathological examination or by PET-CT scan, and if the patient has <grade I toxicity. This particular approach would aid in the design of future protocols wherein REXIN-G is administered in a neoadjuvant/adjuvant setting.

Eligibility (Phase II study)—Patients were required to have recurrent or metastatic osteosarcoma that failed standard chemotherapy. Histologic or cytologic confirmation at diagnosis or recurrence was required. Patients were required to have an ECOG performance score of 0-1 and adequate hematologic, hepatic, and kidney function.

Exclusion criteria included HIV, HBV or HCV positivity, clinically significant ascites, medical, or psychiatric conditions that could compromise successful adherence to the protocol, and unwillingness to employ effective contraception during treatment with REXIN-G and for four weeks following treatment completion. The Western Institutional Review Board approved the protocol and informed consent was obtained from all study participants.

Pre-treatment Evaluation and Follow-up Studies (Phase II study)—Pre-treatment evaluation included history, physical exam, hematology group, chemistry group, assessment of coagulation including prothrombin time (PT), INR, and activated partial thromboplastin time (APTT), testing for HIV, HBV and HCV, imaging evaluation to include FDG/PET-CT scan, EKG and chest x-ray. All patients had a complete blood count and serum chemistry panel performed weekly. In addition, toxicity was assessed before each vector infusion, and before beginning an additional treatment cycle. Efficacy assessment with imaging studies was also performed at the end of 6 weeks or before starting an additional treatment cycle. Patient serum was tested for presence of vector antibodies at 6 weeks and before each treatment cycle. Patient had peripheral blood mononuclear cells collected for assessment of vector DNA integration at the end of 6 weeks and before each treatment cycle. In addition, real-time PCR to detect the presence of replication competent retrovirus (RCR) in peripheral blood mononuclear cells was performed at the end of 6 weeks and before each treatment cycle.

Adaptive Design (Phase II study)—Each treatment cycle was 6 weeks, consisting of 4 weeks treatment and 2 weeks rest period. The following 3 vector dose levels were employed: Dose Level I=1×10¹¹ cfu IV twice a week for 4 weeks; Dose Level II=1×10¹¹ cfu IV three times a week for 4 weeks; Dose Level III=2×10¹¹ cfu IV three times a week for 4 weeks. Treatment cycles were repeated if the patient had Grade I or less toxicity, regardless of the imaging results. To gain better control of tumor growth, intra-patient dose escalation to Dose Level III was allowed (after discussion with the FDA) if disease progression or a disease-related adverse event occurred. Diphenhydramine was given as pre-medication at a dose of 12-50 mg, either intravenously or orally. Tylenol 500 mg p.o., hydrocortisone 50-100 mg IV, and meperidine 25-50 mg IV were prescribed if a hypersensitivity reaction occurred. All patients received clinical lots with a potency of 5×10⁹ cfu/mL. After one or more treatment cycles, the principal investigator may recommend surgical debulking or complete surgical removal. If residual disease is present either by histopathological examination or by PET-CT scan, repeat treatment cycles may be given 4 weeks after surgery, if the surgical incision has healed, and if the patient has <grade I toxicity.

Statistical Methods (Phase II study)—Efficacy information were summarized for each dose as the number and percentage in each of the categories CR, PR, SD, and PD based on the International PET Criteria. The number and percentage achieving any favorable response (defined as CR, PR, or SD and designated as over-all response or OR) at 6 and 12 weeks and at each follow-up PET-CT scan were tabulated. In addition, information is reported for the following endpoints: over-all response rates (CR, PR or SD), progression-free survival and over-all survival. Patients are for survival beyond the one-year evaluation period. All responses are reported. Response rates are reported both as the percentage of eligible patients enrolled in the study (intent-to-treat analysis) and as the percentage of evaluable patients (i.e., eligible patients who finish the treatment course) (“as treated” analysis); 95% confidence intervals for the response rates will be estimated. Survival and time to failure will be summarized with Kaplan-Meier plots. Correlations among extent of tumor burden, tumor response, and dose level were also evaluated.

In the FDA-approved Phase II study, we requested accelerated approval based on completion of this single arm study in 20-30 patients with recurrent or metastatic osteosarcoma who are refractory to known therapies. The endpoint of this Phase II trial would be the percent of over-all positive responses in a single study arm in comparison to historical information. The rapid tumor progression and limited patient survival for patients at an advanced state of disease will be documented. The number of patients needed is a function of the over-all response rate (OR, defined as CR, PR, or SD). Sample sizes are shown in the table below for a comparison of the observed OR rate in patients who are treated with REXIN-G after failure on standard treatment, with 5%, the assumed OR rate in patients who have failed standard treatment and receive no further treatment. We assume a one-sided exact test with significance level 0.025, 80% power, and a range of OR rates in study patients. Duration of and degree of the over-all positive responses would be critical in weighing the approvability of the agent based on the single arm study. Also under consideration would be a median progression-free survival of greater than one month, median over-all survival of greater than 6 months, and avoidance of cytotoxic chemotherapy. Frequency tables, graphs, and summary statistics were used to describe patient characteristics and outcome data. In addition, Kaplan-Meier methodology (Kaplan & Meier 1958) was used to describe the distribution of over-all survival.

Response/Toxicity Criteria (Phase II study)—Response was evaluated using International PET criteria and also RECIST and CHOI criteria according to the FDA-approved protocol. Further, response was evaluated by histopathologic examination of tumor specimens obtained from surgical resection/debulking procedures. Positive responses to REXIN-G treatment are indicated by (i) complete response (CR), partial response (PR) or stable disease (SD) by RECIST and/or International PET criteria, (ii) progression-free survival (PFS) of greater than one month, (iii) over-all survival of 6 months or greater and (iv) histologic findings of greater than 50% tumor necrosis, and presence of calcification and/or fibrosis in tumors.

Toxicity was graded using the National Cancer Institute Common Terminology Criteria Version 3.0. Response was evaluated by FDG/PET/CT scan performed at baseline and following each treatment cycle. Tumor response was evaluated using the NCI RECIST criteria (Therasse et al. 2000) and the International PET criteria. Over-all evaluation of response/toxicity criteria was conducted by the principal investigator.

Results: Single-Agent-Efficacy Study in Osteosarcoma

TABLE 10
Phase II Osteosarcoma (BB-IND# 11586): Treated Analysis

| | Median | | | | | |
|---|---|---|---|---|---|---|
| | Estimated | Response | | | Median PFS | |
| Dose | Tumor | by RECIST | Response | Response | by RECIST | Median OS |
| Level (n) | Burden | or Histopath | by PET | by CHOI | (months) | (months) |
| I-III (17) | 22 × 10e9 | 1CR, 9D, | 1CR, 3PR, | 1CR, 3PR, 11SD | 4 | 8 |
| 1 × 10¹¹ | cancer | 7PD | 8SD, 5PD | 2PD | | 35% one- |
| cfu BIW 2 × | cells | One surgical | | | | year |
| 10¹¹ cfu | | remission | | | | survival rate |
| TIW | | sustained for | | | | 29% 2-year |
| | | 2 years | | | | survival rate |

CR = Complete response;
PR = Partial response;
SD = Stable disease;
PD = Progressive disease;
ND = Not determined;
lesion too small to be determined by CHOI;
BIW = Two times a week;
TIW = Three times a week

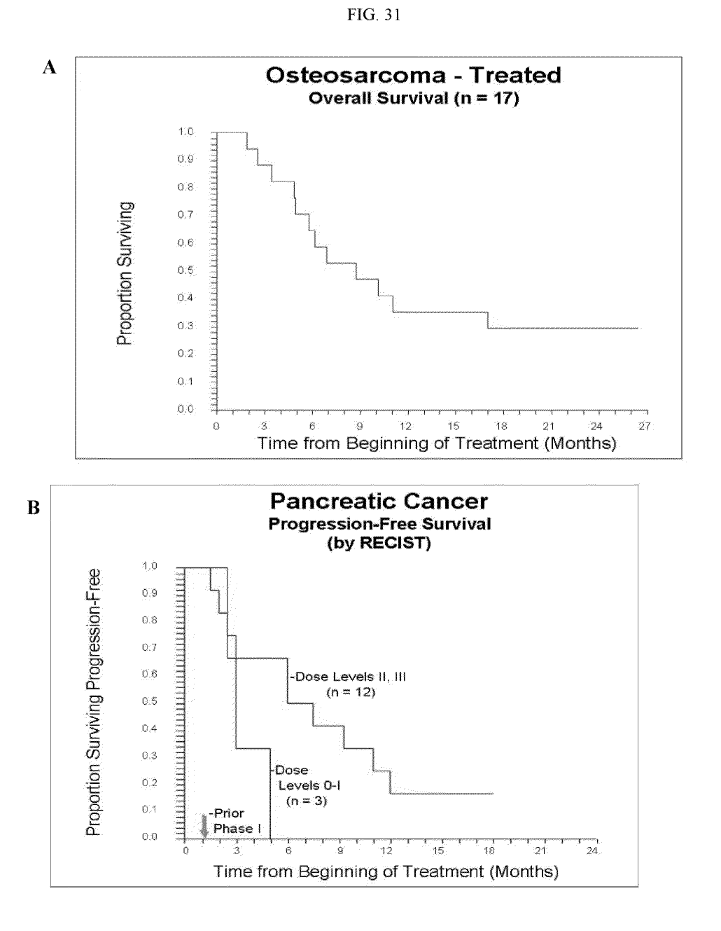

A total of 22 patients were started on REXIN-G, 5 of whom had <1 treatment cycle or did not return for evaluation; Median OS was 6.5 months, 27% one-year survival rate, and 23% two-year survival rate in this intention-to-treat population. FIG. 31A shows the efficacy data on 17 evaluable patients. Using standard RECIST, 10/17 (59%) evaluable patients had a complete surgical response or stable disease, while using International PET criteria, 4/17 patients had complete response or partial responses, and 8/17 patients had stable disease, totaling 71% of patients having partial responses or stable disease. Using CHOI criteria, 4/17 had complete or partial responses and 11/17 had stable disease totaling 88% of patients having complete or partial responses or stable disease. Therefore, tumor responses were significantly higher in the REXIN-G-treated group compared to those expected of historical controls (with ≦5% having a positive response if untreated; p<0.025). Median progression-free survival was 4 months, and overall survival was 8 months (6.5 months for all 22 enrolled patients).

Conclusions of the Phase II Study of REXIN-G in Osteosarcoma: The objectives of the confirmatory Phase II study for osteosarcoma have been met, wherein tumor responses by RECIST of 1 CR/9 SD of 17 evaluable patients (59%; 95% confidence interval, 33-82%), a median PFS of 4 months and a median overall survival of 8 months in patients treated with at least 1 cycle of REXIN-G. For all enrolled patients, median overall survival was 6.5 months. Taken together, the results of two independent well-defined Phase I/II study for sarcoma (three of which were osteosarcoma patients) and Phase II study for osteosarcoma suggest that REXIN-G may help control tumor growth, and may possibly improve progression-free and overall survival times in chemotherapy-resistant sarcoma and osteosarcoma, thus hopefully providing the required elements for accelerated approval for osteosarcoma.

Phase I/II Pancreatic CA: Analysis of efficacy includes evaluable patients up to Dose Level III as shown in Table 11.

TABLE 11
Efficacy data on evaluable patients according to dose level

| | Median | | | | Median | |
|---|---|---|---|---|---|---|
| | Tumor | Tumor | Tumor | Tumor | PFS by | |
| Dose Level (n) | Burden, | Response | Response | Response | RECIST | Median OS |
| (n = 15) | ×10e9 cells | by RECIST | by PET | by CHOI | (months) | (months) |
| 0-I (3) | 18.8 | 3SD | 1PR, 2SD | 1PR, 2SD | 3 | 4.3 |
| Intrapatient dose | | | | | | 0% one-year |
| escalation | | | | | | survival |
| 1 × 10¹¹ cfu BIW- | | | | | | |
| 1 × 10¹¹ cfu TIW | | | | | | |
| II (6) | 15.1 | 1PR, 5SD | 1PR, 5SD | 2PR, 4SD | 7.6 | 9.2 |
| 2 × 10¹¹ cfu TIW | | | | | | 33% one- |
| | | | | | | year |
| | | | | | | survival |
| III (6) | 31.5 | 1CR, 1 PR, | 1CR, 2PR | 1CR, 2PR | 6.8 | 9.3 |
| 3 × 10¹¹ cfu TIW | | 4SD | 3SD | 1SD, 2ND | | 33% one- |
| | | | | | | year |
| | | | | | | survival |

CR = Complete response;
PR = Partial response;
SD = Stable disease;
PD = Progressive disease;
ND = Not determined;
lesion too small to be determined by CHOI;
BIW = Two times a week;
TIW = Three times a week
*20 patients were started on REXIN-G, 5 of whom had <1 treatment cycle or did not return for evaluation; Median OS was 2.6 months for 6 patients at Dose Level 0-I and 9.3 months for 7 patients at Dose Level II and 7.5 months for 7 patients at Dose Level III; 29% one-year survival for both Dose Levels II and III.

A total of 20 patients were started on REXIN-G, 5 of whom had <1 treatment cycle or did not return for evaluation; Median OS was 2.6 months for 6 patients at Dose Level 0-I and 9.3 months for 7 patients at Dose Level II and 7.5 months for 7 patients at Dose Level III. The International PET Criteria and CHOI criteria appear to be more sensitive indicators of response to REXIN-G in terms of detecting partial responses.

Using the one-sided Fisher Test, we compared tumor control responses (by RECIST) in this advanced Phase I/II study (n=15 responses: 1 CR, 2 PR, 12 SD) with those in the prior Phase I study (1 SD, 11 PD, Galanis et al. 2008). With “tumor control response” designated as CR, PR, or SD, the proportions are 15/15 for the current study and 1/12 in the prior study, with p<0.0001 by the one-sided Fisher test. These data indicate a dose response relationship between tumor control response and REXIN-G dosage.

As shown in FIG. 31B, Kaplan-Meier analysis suggests a trend toward a dose-response relationship between progression-free survival (PFS) and REXIN-G dosage. Progression-free survival data from a prior Phase I (C03-101) and the Phase I/II studies (C07-105) are displayed on a Kaplan Meier plot. Proportion of patients surviving progression-free are plotted on the vertical axis as a function of time from beginning of treatment, plotted on the horizontal axis. Note: the blue arrow points to the median PFS of ˜1 month (32 days) of patients treated in the prior Phase I Safety Study, using lower doses of REXIN-G. Prior Phase I study used doses of 0.75-1.5×10¹⁰ cfu for 14-20 doses; Advanced Phase I/II: Dose 0-I=1×10¹¹ cfu two or three times a week; Dose II-III: 2-3×10¹¹ cfu three times a week for 12 doses wherein treatment cycles were repeated if there was Grade 1 or less toxicity. Progression-free survival rates of patients with pancreatic cancer Overall survival data for the Intention-to-Treat population are displayed on a Kaplan Meier plot. The proportion of patients surviving are plotted on the vertical axis as a function of time from beginning of treatment, plotted on the horizontal axis. Similarly, Cox regression analysis and Kaplan Meier analysis shows a dose-response relationship between overall survival and REXIN-G dosage (p=0.03; n=20)

Analysis of REXIN-G Efficacy in Pancreatic Cancer—The clinical data suggest that REXIN-G exhibits significant anti-tumor activity, and may help control tumor growth and improve overall survival in patients with chemotherapy-resistant pancreatic cancer.

Phase I/II Breast Cancer (Analysis of Efficacy) N=20

TABLE 12
Interim Analysis of REXIN-G Efficacy in Breast Cancer

| | Median | | | | |
|---|---|---|---|---|---|
| | Estimated | | | | |
| | Tumor | | | | |
| | Burden, | Median | | | |
| | 1 × 10e9 | Response | PFS by | Median | One |
| Dose Level | cancer | By | RECIST | OS | Year |
| (n) | cells | RECIST | Months | Months | Survival |
| 0-IV (20) | 31 | 14SD, | 3 | >12 | 65% |
| 1-4 × 10e11 | | 4PD, 2^{ND} | >12 months | | |
| cfu BIW-TIW | | 70% | in 2 patients | | |
| | | Tumor | with bone | | |
| | | Control | metastases | | |
| | | Rate | only | | |

Analysis of efficacy in Breast Cancer: The clinical data indicates that REXIN-G may help control tumor growth and possibly help prolong overall survival in chemotherapy-resistant breast cancer.

Vector Safety Studies for Phase I/II and Phase II Protocols—The vector used in the clinical protocols is the REXIN-G retrovector. Potential risks, hazards, and discomforts of retroviral gene delivery include the development of replication-competent retrovirus, dissemination of the REXIN-G vector, insertional mutagenesis/risk of cancer, and development of vector-neutralizing antibodies. These risks are low with the REXIN-G product for the following reasons: 1) Development of replication competent retrovirus (RCR): The incidence of replication-competent retrovirus would be unlikely in a transient plasmid co-transfection system wherein the murine-based retroviral envelope construct, the packaging construct gag pol, and the retroviral vector are expressed in separate plasmids driven by their own promoters. Further, the clinical vector has been tested negative for RCR using validated RCR assays that are in compliance with U.S. FDA guidance/regulations. 2) Dissemination of the REXIN-G vector: Retroviral vectors generated from human cell lines are relatively resistant to inactivation by human complement. Therefore, the infusion of REXIN-G into the systemic circulation would not result in immediate inactivation. However, the REXIN-G vector particles seek out and accumulate in cancerous lesions, and are expected to quickly bind to exposed collagen in the vicinity of target cancer cells. Vectors binding to non-dividing normal cells will most likely be lost, since a built-in safety feature of retroviral vectors is that they integrate only in actively dividing cells. And since collagen is not normally exposed in the circulation, there would only be a small risk of injury to proliferating cells in non-target organs. 3) Insertional mutagenesis/risk of cancer: In the application of gene therapy per se, where a corrective gene is inserted ex vivo into harvested cells, which are then selected, expanded, and engrafted back into patients, ostensibly to produce a long-lasting biochemical correction, vector concerns necessarily persist. In contrast, in the application of genetic medicine for cancer, the gene delivery system was designed to be selective and ablative; thus, the vector is engineered to be “cell inactivating” (CIN). [Note: SIN (self-inactivating) MLV-based vectors developed to date suffer from low titers, repair of the SIN deletion, and negative effects on gene transfer efficiency (Anson, 2004), all of which tend to confound the efficacy of prospective cancer treatments. Moreover, the functional aspects of tumor targeting, including the Epeius “pathotropic” envelope, the choice of a growth-associated cell cycle control knock-out gene, and the basic requirement of cell proliferation for MLV vectors integration, act in concert to improve the safety profile of the gene delivery system to minimize the risk of insertional mutagenesis. 4) Development of vector neutralizing antibodies: The stealth nature and low immunogenicity of the REXIN-G retrovector enables repeated intravenous infusions with less concern for the development of vector-directed antibodies.

To further address these vector safety concerns, clinical toxicity and vector-related safety studies using the REXIN-G vector have been conducted in which the vector was infused intravenously either through a peripheral vein or a central line. Correlative laboratory analysis was performed in the Epeius Biotechnologies Quality Control Unit, using standard operating procedures in compliance with good laboratory practices. In this section, we report on patients' clinical toxicity, hematology, metabolic and chemistry profiles, the results of testing for anti-vector antibodies in patient serum, and testing for presence of replication competent retrovirus (RCR) and vector DNA integration in peripheral blood lymphocytes.

Clinical Toxicity—Clinical toxicity, hematology, metabolic and chemistry profiles of patients are reported according to the NCI Common Terminology Criteria for Adverse Events; CTCAE version. 3). The results of safety/toxicity studies are listed in Tables 13-17.

TABLE 13
USA (BB-IND # 11586)
IND# 11586 Phase I/II Pancreatic CA
Adverse Events by Dose Level and Grade Related to Study Therapy

| | | | | | | | | | |
|---|---|---|---|---|---|---|---|---|---|
| | | Grade 1 | | Grade 2 | | Grade 3 | | Grade 4 | |
| Dose | | (Total | No. | (Total | No. | (Total | No. | (Total | No. |
| Level | Adverse Event | No.) | Unresolved | No.) | Unresolved | No.) | Unresolved | No.) | Unresolved |

| | | | | |
|---|---|---|---|---|
| I | Anorexia | | 1 | |
| N = 3 | Flushing | 1 | | |
| | Nausea | 1 | | |
| | Fever | 1 | | |
| | Abdominal | | 1 | |
| | distention | | | |
| | Insomnia | 1 | | |
| | Diarrhea | 1 | | |
| II | Elevated AST | | | 1 |
| N = 6 | Elevated ALT | | | 1 |
| | Hypermagnesemia | 1 | | |
| | Elevated Alk | 1 | | |
| | Phos | | | |
| III | Diarrhea | 1 | | |
| N = 3 | Nausea | 1 | | |

Note:
The Grade III adverse event occurred in one patient who took 1000 mg acetaminophen daily. Discontinuation of acetaminophen allowed resumption of REXIN-G without recurrence of adverse event, indicating that the Grade III event was due to intake of large doses of acetaminophen.

TABLE 14
USA (BB-IND# 11586)
IND# 11586 Phase I/II Pancreatic CA
Adverse Events by Dose Level and Grade Related to Study Therapy

| | | | | | | | | | |
|---|---|---|---|---|---|---|---|---|---|
| | | Grade 1 | | Grade 2 | | Grade 3 | | Grade 4 | |
| Dose | Adverse | (Total | No. | (Total | No. | (Total | No. | (Total | No. |
| Level | Event | No.) | Unresolved | No.) | Unresolved | No.) | Unresolved | No.) | Unresolved |

| | | | |
|---|---|---|---|
| 0 | None | | |
| N = 6 | | | |
| I-II | Chills | 1 | |
| N = 7 | Fatigue | 2 | 1* |
| | Headache | 1 | |
| III | None | | |
| N = 9 | | | |

*Later attributed to progressive disease

TABLE 15
USA (BB-IND# 11596)
IND# 11586 Phase I/II Sarcoma
Adverse Events by Dose Level and Grade Related to Study Therapy

| | | | | | | | | | |
|---|---|---|---|---|---|---|---|---|---|
| | | Grade 1 | | Grade 2 | | Grade 3 | | Grade 4 | |
| Dose | Adverse | (Total | No. | (Total | No. | (Total | No. | (Total | No. |
| Level | Event | No.) | Unresolved | No.) | Unresolved | No.) | Unresolved | No.) | Unresolved |

| | | | | |
|---|---|---|---|---|
| 0 | Chills | 1 | | |
| N = 6 | Fatigue | 1 | 1 | 1 |
| I-II | Presyncope | 1 | | |
| N = 14 | | | | |
| III | None | | | |
| N = 8 | | | | |
| IV | None | | | |
| N = 8 | | | | |

TABLE 16
USA (BB-IND# 11586)
IND# 11586 Phase I/II Breast CA
Adverse Events by Dose Level and Grade Related to Study Therapy

| | | | | | | | | | |
|---|---|---|---|---|---|---|---|---|---|
| | | Grade 1 | | Grade 2 | | Grade 3 | | Grade 4 | |
| Dose | Adverse | (Total | No. | (Total | No. | (Total | No. | (Total | No. |
| Level | Event | No.) | Unresolved | No.) | Unresolved | No.) | Unresolved | No.) | Unresolved |

| | | |
|---|---|---|
| 0 | None | |
| N = 3 | | |
| I-II | Chills | 1 |
| N = 4 | Itchiness | 1 |
| III | None | |
| N = 7 | | |
| IV | None | |
| N = 6 | | |

TABLE 17
USA (BB-IND# 11586)
IND# 11586 Phase II Osteosarcoma
Adverse Events by Dose Level and Grade Related to Study Therapy

| | | | | | | | | | |
|---|---|---|---|---|---|---|---|---|---|
| | | Grade 1 | | Grade 2 | | Grade 3 | | Grade 4 | |
| Dose | Adverse | (Total | No. | (Total | No. | (Total | No. | (Total | No. |
| Level | Event | No.) | Unresolved | No.) | Unresolved | No.) | Unresolved | No.) | Unresolved |

| | | | |
|---|---|---|---|
| I-II | Photophobia | 1 | |
| N = 22 | Fatigue | 2 | 1 |

* Later attributed to progressive disease

Marketing Experience—REXIN-G gained accelerated approval from the Philippine FDA in December 2007, and is a registered product as an anti-cancer drug for all solid malignancies that have failed standard chemotherapy in the Philippines. Post-marketing monitoring shows no report of serious drug-related adverse events. REXIN-G has been used for compassionate reasons in Japan, Spain, India and Chile and there are no reports of drug-related adverse events in these countries. REXIN-G is not approved in the United States, EMEA nor RoW (other than the Philippines) and has no post-marketing experience in these countries.

Summary of Data and Guidance for the Investigator—The results of four concurrent advanced Phase I/II and Phase II studies evaluating the safety and efficacy of REXIN-G in metastatic pancreas cancer, sarcoma, breast cancer and osteosarcoma, respectively, provide evidence that support the overall safety and potential dose-dependent efficacy of REXIN-G in patients who have failed standard chemotherapy. Progressive stepwise dose-escalations proceeded beyond that of the initial low-dose Phase 1 safety study (Galanis et al. 2008)—in which repeated intravenous infusions yielded no dose-limiting toxicities—to higher, more effective levels where evidence of single-agent efficacy was achieved (Chawla et al. 2009).

The advanced Phase I/II study of intravenous REXIN-G in metastatic gemcitabine-resistant pancreas cancer showed a significant dose response relationship between overall survival and REXIN-G dosage to a level of 0.03 by log rank test in the Intention-to Treat population. Notably, a median survival of 9.2 months and a one-year survival of 29% in the high dose cohorts were shown (Chawla et al., 2009). Similarly, the adaptive Phase I/II study of intravenous REXIN-G in bone and soft tissue sarcoma demonstrated a significant dose response relationship between progression-free survival/overall survival and REXIN-G dosage using the log rank test ((p=0.02 and 0.005 respectively; Chawla et al. 2009). The favorable tumor responses shown by PET-CT scan and potential survival benefits of REXIN-G were also observed in a Phase II study of patients with osteosarcoma who had failed known therapies (Chawla et al. 2009b). The importance of corroborative analysis using International PET Criteria and CHOI criteria with standard RECIST was emphasized in these studies.

In the Phase I/II study of REXIN-G in metastatic breast cancer that failed anthracycline and taxane therapy, tumor control rates of 70% and 65% one-year survival in 20 patients were observed. These data are encouraging because a similar one-year survival time has been reported for paclitaxel when given as first line treatment for metastatic breast cancer.

The absence of dose limiting toxicity in all four clinical trials involving ˜100 patients provide evidence in support of the unique safety of REXIN-G. No vector neutralizing antibodies, no vector DNA integration and no replication competent retrovirus were detected in REXIN-G-treated patients' serum and DNA from peripheral blood lymphocytes up to one year of continued REXIN-G treatment (Chawla et al., 2009). These results would allay retrovector safety concerns by regulatory authorities. Finally, it is relevant to note that REXIN-G has received Orphan Drug designation for pancreas cancer, soft tissue sarcoma and osteosarcoma based on the plausible demonstrations of safety and efficacy as an effective treatment for these serious and life threatening illnesses which represent unmet medical needs.

### Example 20

### Phase I/II Evaluation of Safety and Efficacy of Pathotropic Nanoparticles Bearing a Dominant Negative Cyclin G1 Construct (REXIN-G) as Intervention for Recurrent or Metastatic Sarcoma

The primary objective of this study was to determine the dose-limiting toxicity (DLT) and maximum tolerated dose (MTD) of REXIN-G administered as intravenous infusions. The secondary objectives of this study were to evaluate the potential of REXIN-G for evoking an immune response, recombination events, and unwanted vector integration in nontarget organs, and to identify an objective tumor response to intravenous REXIN-G.

This was an open label, single arm, dose-seeking study that incorporated a modification of the standard Cohort of 3 design combined with a Phase II efficacy phase. Treatment with REXIN-G comprised 6-week cycles that encompassed 4 weeks of treatment, followed by 2 weeks of rest. Five dose levels were planned, beginning at 1.0×10¹¹ cfu given by intravenous (i.v.) infusion two times per week. Three patients were to be treated at each dose level with expansion to 6 patients per cohort if DLT was observed in any 1 of the first 3 patients at each dose level.

The MTD was defined as the highest dose in which 0 of 3 or ≦1 of 6 patients experienced a DLT, with the next higher dose level having at least 2 patients who experienced a DLT.

A DLT was defined as any National Cancer Institute Common Toxicity Criteria for Adverse Events (CTCAE) Grade 3, 4, or 5 adverse event (AE) considered possibly, probably, or definitely related to the study drug, excluding the following: Grade 3 absolute neutrophil count lasting <72 hours; Grade 3 alopecia; or any Grade 3 or higher incident of nausea, vomiting, or diarrhea in a patient who did not receive maximal supportive care.

For the Phase II part of the study, patients who had no toxicity or in whom toxicity had resolved to Grade 1 or less could receive additional cycles of therapy. Protocol Amendments I and II permitted an intra-patient dose escalation up to Dose Level II for patients who had no toxicity or in whom toxicity had resolved to Grade 1 or less, once safety had been established at the higher dose level. Additionally, each cohort also could be expanded to 6 or 7 patients if significant biologic activity was noted at each dose level. The principal investigator was allowed to recommend surgical resection/debulking after at least one treatment cycle has been completed. Response was evaluated first using RECIST (Therasse et al., 2000). Additional evaluations used the International PET criteria (Young et al., (1999) Eur. J. Cancer 35:1773-1782) and a modified RECIST as described by Choi et al., (2007) J. Clin. Oncol. 25:1753-1759. Safety and efficacy analyses were conducted by the Principal Investigator.

36 patents were enrolled (including protocol exemptions and premature terminations). The Intent-to-Treat (ITT) Safety Population was defined as all patients who received at least one infusion of REXIN-G and included 36 patients (used for safety and overall survival). The Modified Intent-to-Treat (mITT) Efficacy Population was defined as all patients who received at least one cycle (4 weeks) of REXIN-G and had a follow-up PET CT scan and included 33 patients (used for response, progression-free survival (PFS) and overall survival (OS)). Gender and race of enrolled subjects are shown in Table 18.

TABLE 18
Patients Enrolled, According to Race and Gender

| | White, | Black, | | Asian, | | |
|---|---|---|---|---|---|---|
| | not of | not of | | or | | |
| | Hispanic | Hispanic | | Pacific | | |
| Gender | Origin | Origin | Hispanic | Islander | Unknown | Total |
| Male | 16 | 0 | 2 | 1 | 0 | 19 |
| Female | 16 | 1 | 0 | 0 | 0 | 17 |
| Total | 32 | 1 | 2 | 1 | 0 | 36 |

Dose Level 0=1×10¹¹ cfu twice per week (BIW); Dose Level I=1×10¹¹ cfu three times per week (TIW); Dose Level II=2×10¹¹ cfu TIW; Dose Level III=3×10¹¹ cfu TIW; Dose Level IV=4×10¹¹ cfu TIW.

Of the 36 enrolled and treated patients, 6 were treated at Dose Levels 0-I, 7 were treated at Dose Levels I-II, 7 were treated at Dose Level II, 8 were treated at Dose Level III, and 8 were treated at Dose Level IV. Thirty-three patients received at least one complete cycle (4 weeks) of treatment and had a follow-up PET-CT scan and were considered evaluable for efficacy. By RECIST, 22 patients had SD and 11 had PD. By International PET criteria, 9 patients achieved a PR, 21 had SD, and 3 had PD. By the modified RECIST criteria of Choi et al., 8 patients achieved a PR, 23 had SD, and 2 had PD. The tumor control rates (CR+PR+SD) were 67% (22/33 patients) by RECIST; 91% (30/33) by PET criteria and 94% (31/33) by Choi-modified RECIST. There were more PRs using PET and Choi-modified RECIST indicating that these tools are more sensitive indicators of tumor response to REXIN-G treatment.

A dose-response effect was not apparent for tumor responses nor PFS. However, a dose-response relationship was apparent between overall survival and REXIN-G dose.

Specifically, none of the patients who received the lowest dose of REXIN-G survived one year. In contrast, 28.5% of patients who received Dose Levels I-II were alive one year after REXIN-G treatment initiation, although none of the patients survived two years after REXIN-G treatment initiation. The best survival data was observed in patients who received the highest doses (Dose Levels III-IV) of REXIN-G, with overall survival estimates in the mITT population of 38.5% at one year and 31% at 2 years, compared to 31.2% at one year and 25% at 2 years in the ITT population.

As of the last follow-up on Feb. 25, 2011, 3 patients remained alive for periods ranging from 32 to 37 months from REXIN-G treatment initiation. Two of these 3 patients were treated at Dose Level III and the 1 was treated at Dose Level IV. Responses are summarized in Table 19.

TABLE 19
Summary of Responses

| | Dose Level | | | | | |
|---|---|---|---|---|---|---|
| Category | 0-I | I-II | II | III | IV | ALL |
| mITT Pop. | N = 6 | N = 7 | N = 7 | N = 6 | N = 7 | N = 33 |
| Median tumor | 50.2 | 25.8 | 46.3 | 53.4 | 39.1 | ND |
| burden* | | | | | | |
| Median Cum. Dose^{†} | 14.5 | 64 | 90 | 142.5 | 96 | ND |
| Response | | | | | | |
| RECIST | 3SD; | 4SD; 3PD | 6SD; 1PD | 4SD; 2PD | 4SD; | 22SD; |
| | 3PD | | | | 3PD | 11PD |
| PET | 1PR; | 1PR; | 4PR; 3SD | 1PR; 4SD; | 2PR; | 9PR; |
| | 4SD; | 5SD; 1PD | | 1PD | 5SD | 21SD; 3PD |
| | 1PD | | | | | |
| Choi | 1PR; | 3PR; | 3PR; 4SD | 6SD | 1PR; | 8PR; |
| | 5SD | 3SD; 1PD | | | 5SD; | 23SD; 2PD |
| | | | | | 1PD | |
| Median PFS (mo) | | | | | | |
| RECIST | 1.2 | 3 | 4.5 | 3.0 | 3.0 | ND |
| PET | 2.8 | 4.5 | 6.0 | >3.5 | >3.0 | ND |
| Choi | 4.2 | 4.5 | 6.0 | 3.5 | 3.0 | ND |
| Median OS (mo) | 3.3 | 8.1 | 7.6 | 13.8 | 10.7 | ND |
| % OS | | | | | | |
| 1 year | 0% | 28.5% | | 38.5% | | ND |
| 2 years | 0% | 0% | | 31.0% | | ND |
| ITT Pop. | N = 6 | N = 7 | N = 7 | N = 8 | N = 8 | N = 36 |
| Median OS (mo) | 3.3 | 8.1 | 7.6 | 6.8 | 9.8 | ND |
| % OS | | | | | | |
| 1 year | 0% | 28.5% | | 31.2% | | ND |
| 2 years | 0% | 0% | | 25.0% | | ND |
| # Alive | 0 | 0 | 0 | 2 | 1 | 3 |

*Number of cells = number shown × 10⁹.
^{†}Number of cfu = number shown × 10¹¹.

There was no dose-limiting toxicity at any dose level. Unrelated adverse events were reported for all patients, but the number of events was low (in most cases 1 or 2 occurrences per adverse event), and most were Grade 1 or 2. Eight patients experienced related adverse events; all were mild or moderate in severity. Twenty patients experienced serious adverse events (SAEs), all of which were deemed not related to the study drug.

All 36 patients experienced one or more nondrug-related nonserious AEs. The majority of these unrelated AEs were Grade 1 or 2. No relationships were apparent between AEs and dose of REXIN-G. In fact, there were more non-related Grade 3 adverse events in patients who received lower doses of REXIN-G, indicating that the adverse events were related to the cancer. The most frequent Grade 3, nonserious, unrelated adverse events were anemia (10 patients), hypokalemia (5 patients), and hyponatraemia (5 patients). Abdominal pain, blood alkaline phosphatase increased, hypoalbuminaemia, and hypocalcaemia were reported in 3 patients each. Hyperbilirubinaemia, musculoskeletal chest pain, respiratory acidosis, and respiratory failure were reported in 2 patients each. All other Grade 3 AEs were reported in only 1 patient each, and all were due to disease progression.

Eight of the 36 treated patients each experienced 1 drug-related adverse event. These 8 events comprised chills (2 patients), fatigue (5 patients), and hypersensitivity (1 patient). All study drug-related AEs were nonserious and Grade 1 or 2 in severity. No dose trends were apparent for these related AEs.

Twenty of the 36 treated patients were reported to have had SAEs. None of the SAEs were related to the study drug.

As of Feb. 25, 2011, 33/36 patients from the have died. None of the deaths were considered related to REXIN-G. The cause of death was progressive disease in 30 of the 33 patients who died. Causes of death in the other 3 patients who died were iatrogenic esophageal and aortic bleeding from a stent procedure, sepsis with disseminated intravascular coagulation, and post-operative complication (arrhythmia and dehydration).

Vector-related safety parameters also indicated no adverse effects of REXIN-G: three patients tested weakly positive for antibodies to gp70—in each case, the response was transient and this was not associated with detection of vector neutralizing antibodies; no patient tested positive for any of the following: vector neutralizing antibodies, replication-competent retrovirus in peripheral blood lymphocytes (PBLs); or vector integration into genomic DNA of PBLs.

This study demonstrates that REXIN-G is safe and well-tolerated with minimal toxicity at the prescribed doses. The high tumor control rates (67% by RECIST, 91% by PET, and 94% by Choi) indicate that REXIN-G has substantial activity in patients with recurrent or metastatic sarcoma who have failed standard chemotherapy. The observation that 8-9 (24-27%) patients were assessed as PRs using the PET or Choi tumor assessment criteria, but not by RECIST suggest that PET and Choi are more sensitive indicators of tumor responses to REXIN-G treatment and RECIST may not be the optimal assessment tool for trials using REXIN-G. Finally, the dose-response relationship between overall survival and REXIN-G dose indicate that REXIN-G may prolong overall survival in chemotherapy-resistant patients with bone and soft tissue sarcoma.

### Example 21

### Phase I/II Evaluation of Safety and Efficacy of Pathotropic Nanoparticles Bearing a Dominant Negative Cyclin G1 Construct (REXIN-G) as Intervention for Recurrent or Metastatic Breast Cancer

The primary objective of this study was to determine the dose-limiting toxicity (DLT) and maximum tolerated dose (MTD) of REXIN-G administered as intravenous infusions. The secondary objectives of this study were to evaluate the potential of REXIN-G for evoking an immune response, recombination events, and unwanted vector integration in nontarget organs, and to identify an objective tumor response to intravenous REXIN-G.

This was an open label, single arm, dose-seeking study that incorporated a modification of the standard Cohort of 3 design combined with a Phase II efficacy phase. Treatment with REXIN-G comprised 6-week cycles that encompassed 4 weeks of treatment, followed by 2 weeks of rest. Five dose levels were planned, beginning at 1.0×10¹¹ cfu given by intravenous (i.v.) infusion two times per week. Three patients were to be treated at each dose level with expansion to 6 patients per cohort if DLT was observed in any 1 of the first 3 patients at each dose level.

The MTD was defined as the highest dose in which 0 of 3 or ≦1 of 6 patients experienced a DLT, with the next higher dose level having at least 2 patients who experienced a DLT.

A DLT was defined as any National Cancer Institute Common Toxicity Criteria for Adverse Events (CTCAE) Grade 3, 4, or 5 adverse event (AE) considered possibly, probably, or definitely related to the study drug, excluding the following: Grade 3 absolute neutrophil count lasting <72 hours; Grade 3 alopecia; or any Grade 3 or higher incident of nausea, vomiting, or diarrhea in a patient who did not receive maximal supportive care.

For the Phase II part of the study, patients who had no toxicity or in whom toxicity had resolved to Grade 1 or less could receive additional cycles of therapy. Protocol Amendments I and II permitted an intra-patient dose escalation up to Dose Level II for patients who had no toxicity or in whom toxicity had resolved to Grade 1 or less, once safety had been established at the higher dose level. Additionally, each cohort also could be expanded to 6 or 7 patients if significant biologic activity was noted at each dose level. The principal investigator was allowed to recommend surgical resection/debulking after at least one treatment cycle has been completed. Response was evaluated first using RECIST (Therasse et al., 2000). Additional evaluations used the International PET criteria (Young et al., (1999) Eur. J. Cancer 35:1773-1782) and a modified RECIST as described by Choi et al., (2007) J. Clin. Oncol. 25:1753-1759. Safety and efficacy analyses were conducted by the Principal Investigator.

20 patents were enrolled. The Intent-to-Treat (ITT) Safety Population was defined as all patients who received at least one dose of REXIN-G and included 20 patients (used for safety and overall survival). The Modified Intent-to-Treat (mITT) Efficacy Population was defined as all patients who received at least one cycle and had a follow-up PET-CT scan and included 18 patients (used for response, progression-free survival (PFS) and overall survival (OS)). Gender and race of enrolled subjects are shown in Table 20.

TABLE 20
Patients Enrolled, According to Race and Gender

| | White, | Black, | | Asian, | | |
|---|---|---|---|---|---|---|
| | not of | not of | | or | | |
| | Hispanic | Hispanic | | Pacific | | |
| Gender | Origin | Origin | Hispanic | Islander | Unknown | Total |
| Male | 0 | 0 | 0 | 0 | 0 | 0 |
| Female | 19 | 0 | 0 | 1 | 0 | 20 |
| Total | 19 | 0 | 0 | 1 | 0 | 20 |

Dose Level 0=1×10¹¹ cfu twice per week (BIW); Dose Level I=1×10¹¹ cfu three times per week (TIW); Dose Level II=2×10¹¹ cfu TIW; Dose Level III=3×10¹¹ cfu TIW; Dose Level IV=4×10¹¹ cfu TIW.

Of the 20 enrolled and treated patients, 7 were treated at Dose Levels 0-II, 7 were treated at Dose Level III, and 6 were treated at Dose Level IV. Seventeen patients received at least one complete cycle (4 weeks) of treatment and had a follow-up PET CT scan and were considered evaluable for efficacy. By RECIST, 13 patients had SD and 4 had PD, with no apparent dose-response relationship, as similar numbers of patients had SD or PD at each dose level. The tumor control rate (CR+PR+SD) by RECIST was 76% (13/17 patients).

PFS by RECIST ranged from 3.5 months at Dose Level 0-I, 1.25 months at Dose Level II and 3 months at Dose Level III, thus no dose-response relationship was apparent. A higher tumor burden was observed for patients in Dose Level III, which may explain the shorter PFS. Of note, two patients with extensive bone metastases only and no visceral involvement (one patient at Dose Level III and one at Dose Level IV) had a PFS of greater than one year, and remain alive more than one year after treatment initiation.

OS was examined in the ITT and mITT population. OS estimates at 1 year was 60% at all dose levels (66% in the mITT population), and 83% at Dose Level IV in the ITT and mITT populations. Eight of 20 patients remained alive for 19 to 43 months from treatment initiation as of the last follow-up on Jun. 24, 2011. Of those remaining alive, 1 was treated at Dose Level 0-II, 2 were treated at Dose Level III, and 5 were treated at Dose Level IV. Responses are summarized in Table 21.

TABLE 21
Summary of Responses

| | Dose Level | | | |
|---|---|---|---|---|
| | 0-II | III | IV | ALL |
| Category | | | | |
| mITT Pop. | N = 6 | N = 6 | N = 6 | N = 18 |
| Median tumor | 33.8 | 73.9 | 31.0 | ND |
| burden* | | | | |
| Median Cum. Dose^{†} | 53 | 54 | 120 | ND |
| Response | | | | |
| RECIST | 5SD; 1PD | 4SD; 1PD | 4SD; 2PD | 13SD; 4PD |
| Median PFS (mo) | | | | |
| RECIST | 3.5 | 1.25 | 3 | ND |
| ITT Pop. | N = 7 | N = 7 | N = 6 | N = 20 |
| Median OS (mo) | 33 | 5.5 | 21.8 | 20 |
| % OS | | | | |
| 1 year | 71.4% | 28.6% | 83.0% | 60% |
| 2 years | 57.1% | 28.6% | 71.4% | 40% |
| # Alive | 1/7 | 2/7 | 5/6 | 8/20 |

*Number of cells = number shown × 10⁹.
^{†} Number of cfu = number shown × 10¹¹.

There were no dose-limiting toxicities at any dose level. Unrelated adverse events were reported for all patients, but the number of events was low (in most cases 1 or 2 occurrences per adverse event), and most were Grade 1 or 2. Related adverse events occurred in 5 patients, and all but one were Grade 1 or 2. Three patients experienced serious adverse events, all of which were deemed not related to the study drug.

All 20 patients experienced one or more nonserious AEs that were considered by the Investigator to be unrelated to the study drug. The majority of unrelated events were Grade 1 or 2.

The most frequent nonserious unrelated Grade 3 AE was vomiting (3 patients). Other Grade 3 AEs that were reported in 2 patients were anemia, nausea, AST increased, alkaline phosphatase increased, and phosphorus increased. All other Grade 3 AEs were reported in only one patient each. No dose trend was apparent.

Five of the 20 treated patients each experienced a total of 8 drug-related adverse events. Three of the 5 patients had 1 drug-related AE each, 1 patient had 2 drug-related AEs and 1 patient had 3 drug-related AEs. These 8 events comprised chills, pruritic, pruritic rash, dry skin, and hot flush in 1 patient each and dysgeusia in 3 patients. All study drug-related AEs were nonserious and Grade 1 or 2 in severity, except for one event of Grade 3 pruritic rash. All of the drug-related AEs occurred in patients treated at Dose Level II or higher and 6 of the 8 events occurred in patients treated at Dose Level III or IV, and were hypersensitivity reactions.

Three of twenty patients were reported to have had serious adverse events which were considered not related to the study drug. These comprised Grade 2 malignant pleural effusion in one patient and Grade 2 pathological fracture in one patient. One patient had 6 SAEs: Grade 4 pulmonary embolism, Grade 4 neutropenia, Grade 4 pyrexia, Grade 4 dyspnoea, Grade 4 respiratory congestion, and Grade 4 Pseudomonas infection. None were related to the study drug. No dose trends were apparent.

As of Feb. 25, 2011, 12/20 patients have died. None of the deaths were considered related to REXIN-G. All deaths were as a result of disease progression.

Vector-related safety parameters also indicated no adverse effects of REXIN-G: no patient tested positive for any of the following: vector neutralizing antibodies, antibodies to gp70, replication-competent retrovirus in peripheral blood lymphocytes (PBLs); vector integration into genomic DNA of PBLs.

The tumor control rate of 76% indicates that REXIN-G may have anti-tumor activity in patients with recurrent or metastatic breast cancer who have failed prior chemotherapy. The 83% OS rate at 1 year for Dose Level IV is promising and suggests a survival benefit over 70% OS in historical controls receiving first-line therapy with paclitaxel (Leo et al., 2009). Of note, two patients with extensive bone metastases only and no visceral involvement had the longest PFS and are alive greater than one year from REXIN-G treatment initiation. No safety issues with REXIN-G were apparent.

### Example 22

### Phase I/II Evaluation of Safety and Efficacy of Pathotropic Nanoparticles Bearing a Dominant Negative Cyclin G1 Construct (REXIN-G) as Intervention for Recurrent or Metastatic Pancreatic Cancer

The primary objective of this study was to determine the dose-limiting toxicity (DLT) and maximum tolerated dose (MTD) of REXIN-G administered as intravenous infusions. The secondary objectives of this study were to evaluate the potential of REXIN-G for evoking an immune response, recombination events, and unwanted vector integration in nontarget organs, and to identify an objective tumor response to intravenous REXIN-G.

This was an open label, single arm, dose-seeking study that incorporated a modification of the standard Cohort of 3 design combined with a Phase II efficacy phase. Treatment with REXIN-G comprised 6-week cycles that encompassed 4 weeks of treatment, followed by 2 weeks of rest. Five dose levels were planned, beginning at 1.0×10¹¹ cfu given by intravenous (i.v.) infusion two times per week. Three patients were to be treated at each dose level with expansion to 6 patients per cohort if DLT was observed in any 1 of the first 3 patients at each dose level.

The MTD was defined as the highest dose in which 0 of 3 or ≦1 of 6 patients experienced a DLT, with the next higher dose level having at least 2 patients who experienced a DLT.

A DLT was defined as any National Cancer Institute Common Toxicity Criteria for Adverse Events (CTCAE) Grade 3, 4, or 5 adverse event (AE) considered possibly, probably, or definitely related to the study drug, excluding the following: Grade 3 absolute neutrophil count lasting <72 hours; Grade 3 alopecia; or any Grade 3 or higher incident of nausea, vomiting, or diarrhea in a patient who did not receive maximal supportive care.

For the Phase II part of the study, patients who had no toxicity or in whom toxicity had resolved to Grade 1 or less could receive additional cycles of therapy. Protocol Amendments I and II permitted an intra-patient dose escalation up to Dose Level II for patients who had no toxicity or in whom toxicity had resolved to Grade 1 or less, once safety had been established at the higher dose level. Additionally, each cohort also could be expanded to 6 or 7 patients if significant biologic activity was noted at each dose level. The principal investigator was allowed to recommend surgical resection/debulking after at least one treatment cycle has been completed. Response was evaluated first using RECIST (Therasse et al., 2000). Additional evaluations used the International PET criteria (Young et al., (1999) Eur. J. Cancer 35:1773-1782) and a modified RECIST as described by Choi et al., (2007) J. Clin. Oncol. 25:1753-1759. Safety and efficacy analyses were conducted by the Principal Investigator.

20 patents were enrolled. The Intent-to-Treat (ITT) Safety Population was defined as all patients who received at least one dose of REXIN-G and included 20 patients (used for safety and overall survival). The Modified Intent-to-Treat (mITT) Efficacy Population was defined as all patients who received at least one cycle and had a follow-up PET-CT scan and included 15 patients (used for response, progression-free survival (PFS) and overall survival (OS)). Gender and race of enrolled subjects are shown in Table 22.

TABLE 22
Patients Enrolled, According to Race and Gender

| | White, | Black, | | Asian, | | |
|---|---|---|---|---|---|---|
| | not of | not of | | or | | |
| | Hispanic | Hispanic | | Pacific | | |
| Gender | Origin | Origin | Hispanic | Islander | Unknown | Total |
| Male | 5 | 0 | 0 | 3 | 0 | 8 |
| Female | 11 | 0 | 0 | 1 | 0 | 12 |
| Total | 16 | 0 | 0 | 4 | 0 | 20 |

Dose Level 0=1×10¹¹ cfu twice per week (BIW); Dose Level I=1×10¹¹ cfu three times per week (TIW); Dose Level II=2×10¹¹ cfu TIW; Dose Level III=3×10¹¹ cfu TIW.

Of the 20 enrolled and treated patients, 6 were treated at Dose Levels 0-I, 7 were treated at Dose Level II, and 7 were treated at Dose Level III. Fifteen patients received at least one complete cycle (4 weeks) of treatment and had a follow-up PET-CT scan and were considered evaluable for efficacy (known as the modified Intent-to-Treat or mITT population) in terms of response, progression-free survival and overall survival.

By RECIST, one patient achieved a CR, two patients had a PR and 12 had SD. A higher tumor burden was observed for patients in Dose Levels II and III compared with Dose Level 0-I. The tumor control rate (CR+PR+SD) by RECIST was 100% (15/15 patients.) Responses were better when assessed using PET criteria or Choi-modified RECIST. By PET, one patient achieved a CR, 4 patients had a PR, and 10 patients had SD. By Choi, one patient had a CR, 5 had a PR and 9 had SD. By RECIST, PRs and CRs occurred only at Dose Levels II and III, suggesting a dose-dependent relationship between REXIN-G dose and response.

PFS by RECIST was 3, 7.6, and 6.8 months at Dose Levels 0-I, II, and III, suggesting a dose-dependent relationship between REXIN-G dose and PFS.

OS estimates in the efficacy evaluable mITT population among the combined group of Dose Levels 0-I was 0% at one year. In contrast, OS estimates in the combined groups Dose Levels II-III were 33.3% at one year and 25% at 2 years. These findings indicate a dose-dependent relationship between REXIN-G dose and overall survival.

OS estimates in the Intent-to-Treat or ITT population (defined as all patients who received at least one dose of REXIN-G) among the combined group of Dose Levels 0-I was 0% at one year. In contrast, OS estimates among the combined group of Dose Levels II-III were 28.5% 1 year and 21.4% at 2 years. Taken together, these findings indicate a dose-dependent relationship between REXIN-G dose and overall survival. Responses are summarized in Table 23.

TABLE 23
Summary of Responses

| | Dose Level | | | |
|---|---|---|---|---|
| | 0-I | II | III | ALL |
| Category | | | | |
| mITT | N = 3 | N = 6 | N = 6 | N = 15 |
| Pop. | | | | |
| Median | 13.5 | 37.1 | 38.0 | ND |
| tumor | | | | |
| burden* | | | | |
| Median | 20 | 70 | 160.5 | ND |
| Cum. | | | | |
| Dose^{†} | | | | |
| Response | | | | |
| RECIST | 3SD | 1PR; 5SD | 1CR; 1PR; 4SD | 1CR; 2PR; 12SD |
| PET | 1PR; 2SD | 1PR; 5SD | 1CR; 2PR; 3SD | 1CR; 4PR; 10SD |
| Choi | 1PR; 2SD | 2PR; 4SD | 1CR; 2PR; 3SD | 1CR; 5PR; 9SD |
| Median | | | | |
| PFS (mo) | | | | |
| RECIST | 3.0 | 7.6 | 6.8 | ND |
| PET | >3.0 | >7.6 | 3.0 | ND |
| Choi | >3.0 | >7.6 | 8.5 | ND |
| Median | 4.3 | 9.2 | 9.2 | ND |
| OS (mo) | | | | |
| % OS | | | | |
| 1 year | 0% | 33.3% | | ND |
| 2 years | | 25.0% | | ND |
| ITT Pop. | N = 6 | N = 7 | N = 7 | N = 20 |
| Median | 2.6 | 9.0 | 7.8 | ND |
| OS (mo) | | | | |
| % OS | | | | |
| 1 year | 0% | 28.5% | | ND |
| 2 years | 0% | 21.4% | | ND |
| # Alive | 0 | 0 | 1 | 1 |

*Number of cells = number shown × 10⁹.
^{†}Number of cfu = number shown × 10¹¹.

There were no dose-limiting toxicities at any dose level. Unrelated adverse events were reported for all patients, but the number of events was low (in most cases 1 or 2 occurrences per preferred term), and most were Grade 1 or 2. Related nonserious adverse events occurred in 7 patients, and all were Grade 1. Thirteen patients experienced serious adverse events, all of which were deemed not related to the study drug.

All 20 patients experienced one or more unrelated nonserious adverse events. The majority of unrelated adverse events were Grade 1 or 2 in severity. Several types of adverse events appeared to be more frequent at higher doses: Anaemia: 2 of 6 patients at Dose Level 0, 2 of 3 patients at Dose Level I, 7 of 7 patients at Dose Level II, and 5 of 7 treated at Dose Level III; Hyperbilirubinaemia: 1 of 6 patients at Dose Level 0, 1 of 3 patients at Dose Level I, and 3 of 7 patients at Dose Level II, and 4 of 7 at Dose Level III; Aspartate aminotransferase: 2 of 6 at Dose Level 0; 1 of 3 at Dose Level I, 4 of 7 at Dose Level II, and 3 of 7 at Dose Level III; and Decreased appetite: 1 of 6 at Dose Level 0; 2 of 3 at Dose Level I 4 of 7 at Dose Level II, and 4 of 7 at Dose Level III.

The most frequent nonserious unrelated Grade 3 AEs were hypoalbuminemia (4 patients) and increased alanine aminotransferase (3 patients). Anemia, hyperglycemia, increased aspartate aminotransferase and hypocalcemia were reported in 2 patients each. Other nonserious unrelated Grade 3 AEs were reported in only 1 patient each. Grade 3 AEs appeared to be more frequent at Dose Level III.

Related adverse events occurred in 7 patients (Table 4) and comprised chills (1 patient), fatigue (2 patients) and headache (1 patient) at Dose Level 2 and fatigue (4 patients) at Dose Level 3. All were Grade 1 and nonserious. There were no serious drug-related AEs.

Twenty-six serious adverse events were reported in 13 patients. None were related to the study drug.

As of Feb. 25, 2011, 19/20 patients have died. None of the deaths were considered related to REXIN-G. The cause of deaths was progressive disease in all but one patient; the cause of death for this patient was sepsis.

Vector-related safety parameters also indicated no adverse effects of REXIN-G: no patient tested positive for any of the following: vector neutralizing antibodies, antibodies to gp70, replication-competent retrovirus in peripheral blood lymphocytes (PBLs); vector integration into genomic DNA of PBLs.

The tumor control rate of 100% indicates that REXIN-G has substantial anti-tumor activity in patients with recurrent or metastatic pancreatic cancer who have failed gemcitabine or gemcitabine-containing chemotherapy. The longer PFS and OS at Dose Levels II and III compared to Dose 0-II are significant for this population. The better responses observed using PET and Choi-modified RECIST suggest that these alternative evaluation methods may be more sensitive indicators of tumor response than RECIST in patients with advanced pancreatic cancer.

### Example 23

### Phase II Evaluation of Safety and Efficacy of Pathotropic Nanoparticles Bearing a Dominant Negative Cyclin G1 Construct (REXIN-G) as Intervention for Recurrent or Metastatic Osteosarcoma

The primary objective of this study was to assess the clinical efficacy of intravenous (IV) REXIN-G in terms of tumor response rates, progression-free survival and over-all survival. The secondary objectives were to evaluate the over-all safety of intravenously administered REXIN-G as evaluated by performance status, toxicity assessment score, hematologic, metabolic profiles, immune responses, vector integration in PBLs and recombination events.

Patients with recurrent or metastatic osteosarcoma considered refractory to known therapies were eligible for this study. Patients received intravenous infusions of REXIN-G two or three times per week for 4 weeks followed by a two-week rest period. Patients were assigned to a dose of 1×10¹¹ cfu BIW if the tumor burden was <10×10⁹ cells or to a dose of 1×10¹¹ cfu TIW if the tumor burden was >10×10⁹ cells. Patients with no toxicity or in whom toxicity had resolved to ≦Grade I could receive additional cycles.

Protocol Amendments I and II permitted intra-patient dose escalation up to 2×10⁹ cfu TIW for patients who had no toxicity or in whom toxicity had resolved to ≦Grade I, once safety had been established at the higher dose level. The principal investigator was allowed to recommend surgical resection/debulking after at least one treatment cycle has been completed. Response was evaluated first using RECIST (Therasse et al., 2000). Additional evaluations used the International PET criteria (Young et al., (1999) Eur. J. Cancer 35:1773-1782) and a modified RECIST as described by Choi et al., (2007) J. Clin. Oncol. 25:1753-1759. Safety and efficacy analyses were conducted by the Principal Investigator.

22 patents were enrolled. The Intent-to-Treat (ITT) Safety Population was defined as all patients who received at least one dose of REXIN-G and included 22 patients (used for safety and overall survival). The Modified Intent-to-Treat (mITT) Efficacy Population was defined as all patients who received at least one cycle and had a follow-up PET-CT scan and included 17 patients (used for response, progression-free survival (PFS) and overall survival (OS)). Gender and race of enrolled subjects are shown in Table 24.

TABLE 24
Patients Enrolled, According to Race and Gender

| | White, | Black, | | Asian, | | |
|---|---|---|---|---|---|---|
| | not of | not of | | or | | |
| | Hispanic | Hispanic | | Pacific | | |
| Gender | Origin | Origin | Hispanic | Islander | Unknown | Total |
| Male | 7 | 5 | 5 | 0 | 0 | 17 |
| Female | 2 | 1 | 2 | 0 | 0 | 5 |
| Total | 9 | 6 | 7 | 0 | 0 | 22 |

Fourteen of the 22 enrolled and treated patients were initially treated at either 1×10e11 BIW or 1×10e11 TIW and then escalated to 2×10e11 cfu TIW, and 8 patients were treated only at 2×10e11 cfu TIW. Seventeen patients received at least one complete cycle (4 weeks) of treatment and had a follow-up PET-CT scan and were considered evaluable for efficacy. By RECIST, 10 patients achieved SD and 7 had PD. The tumor control rate (CR+PR+SD) by RECIST was 59% (10/17 patients). Responses were better when assessed using PET criteria or Choi-modified RECIST (Table 2): by PET, 4 patients achieved a PR, 8 patients had SD, and 5 had PD and by the Choi method, 3 had PRs, 12 had SD, and 2 had PD. Median PFS by RECIST was 3.0 months overall for the efficacy evaluable subset and median OS was 8.7 months for the efficacy evaluable (mITT) patients and OS estimates in this REXIN-G: group were 35.3% at one year, 29.4% at two years and 17.6% at three years. For the ITT population, OS was 6.0 months, and OS estimates in the ITT population were 27.3% at 1 year and 22.7% at 2 years and 13.6% at 3 years. Three patients remained alive for a period ranging from 25 months to 38 months, as of the last follow-up on Feb. 25, 2011. Responses are summarized in Table 25.

TABLE 25
Summary of Responses

| mITT Pop. | N = 17 |
|---|---|
| Median tumor | 25.8 |
| burden* | |
| Median Cum. Dose^{†} | 62.0 |
| Response | |
| RECIST | 10SD; 7PD (59% TCR) |
| PET | 3PR; 9SD; 5PD (70% TCR) |
| Choi | 5PR; 10SD; 2PD (88% TCR) |
| Median PFS (mo) | |
| RECIST | 3.0 |
| PET | 3.0 |
| Choi | 3.0 |
| Median OS (mo) | 8.7 |
| % OS | |
| 1 year | 35.3% |
| 2 years | 29.4% |
| 3 years | 17.6% |
| ITT Pop. | N = 22 |
| Median OS (mo) | 6.0 |
| % OS | |
| 1 year | 27.3% |
| 2 years | 22.7% |
| 3 years | 13.6% |
| # Alive | 3/22 |

*Number of cells = number shown × 10⁹.
^{†}Number of cfu = number shown × 10¹¹.

All 22 patients experienced one or more unrelated nonserious adverse events. The majority of unrelated events were Grade 1 or 2. The most frequent nonserious, unrelated AEs were anemia (16 patients), alkaline phosphatase increased (12 patients), hyperglycaemia (11 patients), hypoalbuminemia, hypoglycaemia, and hypokalemia (9 patients each).

The most frequent nonserious unrelated Grade 3 AEs were anemia (8 patients), hyperglycemia, hypoalbuminemia, and alkaline phosphatase increased (5 patients each), and hypocalcemia (4 patients). Tachycardia, sepsis, hypokalaemia, hypophosphatemia, and chest pain were reported for 3 patients each. Asthenia, fatigue, dehydration, and hypokalemia were reported for 2 patients each.

Related adverse events occurred in 4 patients (all treated at Dose Level II).

Nine patients were reported to have had 16 serious adverse events. Most were Grade 2 or 3 (one SAE was Grade 4). None were related to the study drug.

As of Feb. 25, 2011, 19/22 patients have died. None of the deaths were considered related to REXIN-G. One patient died during the reporting period. The cause of death was progressive disease in all patients.

Vector-related safety parameters also indicated no adverse effects of REXIN-G: no patient tested positive for any of the following: vector neutralizing antibodies, antibodies to gp70, replication-competent retrovirus in peripheral blood lymphocytes (PBLs); vector integration into genomic DNA of PBLs.

The tumor control rate of 59% indicates that REXIN-G has substantial anti-tumor activity in patients with recurrent or metastatic osteosarcoma who have failed all known therapies. The better responses observed using PET and Choi-modified RECIST suggest that these alternative evaluation methods may be more sensitive early tumor response indicators in patients with chemotherapy-resistant osteosarcoma.

### Example 24

### Compassionate Use of REXIN-G for Pancreatic Cancer, Breast Cancer, Sarcoma, Osteosarcoma and Other Solid Malignancies Refractory to Standard Therapy

The patient will receive REXIN-G intravenously at a dose of 2×10¹¹ cfu per dose, five days a week, for 4 weeks. If there is <Grade I toxicity, may continue REXIN-G at a dose of 2×10¹¹ cfu 3 days a week for 8 more weeks. If the patient develops a Grade 3 or greater adverse event (CTCAE Vs 3.0) which appears to be related or possibly related to REXIN-G, the infusion will be held and the patient will be monitored until the toxicity resolves or the patient is stable. The infusion may be considered to be resumed if the toxicity is grade 3 and resolved to grade 1 or less within 24 hours. If the adverse event does not resolve within 72 hours, the study will be held until the data are discussed with the Food and Drug Administration (FDA) and a decision is made whether to continue or terminate the study.

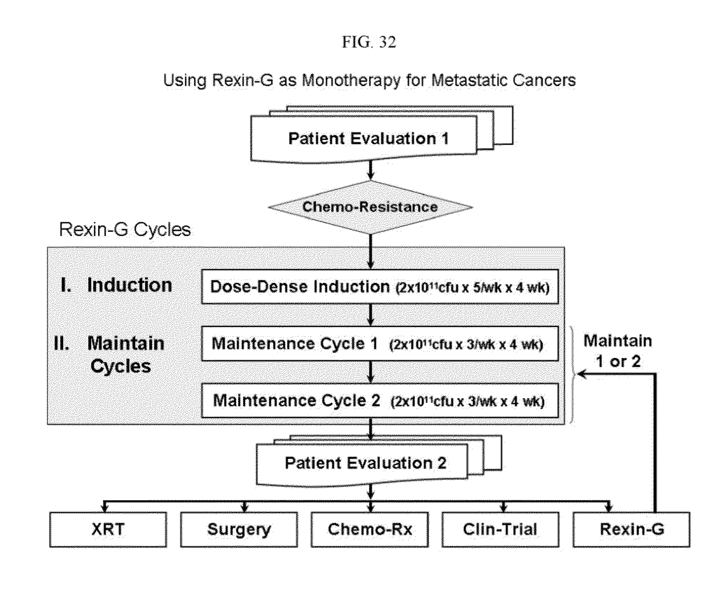

Patients may have additional treatment cycles if they have clinical benefit and have <Grade 1 toxicity. The principal investigator may recommend surgical resection/debulking/biopsy after completion of the 12-week treatment. Patient may resume treatment with REXIN-G for an additional 6 months after surgery. Principal investigator may recommend radiation therapy, resumption of palliative chemotherapy or enrollment in another clinical study upon completion of 12 week treatment (see FIG. 32).

The vector is stored in −80±10° C. freezer until used. Fifteen minutes before infusion, the product is thawed at 32-36° C. waterbath and immediately infused upon thawing.

Patient will receive injections of the REXIN-G vector via a peripheral vein or a central IV line by slow IV injection at 4 ml per minute.

Thirty minutes prior to vector infusion: Acute reaction prophylactic therapy consists of Benadryl (12.5-25 mg) IV push or p.o. and dexamethasone 2 mg p.o.; ranitidine 300 b.i.d. (to prevent stress ulcers from steroid therapy); if allergic reactions develop, hydrocortisone 50-100 mg IV push, and acetaminophen 500 mg p.o. for fever. Post-infusion, non-steroidal anti-inflammatory drugs, such as ibuprofen, may be used prn for pain and/or fever.

### Scheduled Evaluations and Monitoring

Day 0 Baseline Tests (within 2 Weeks Pre-REXIN-G Infusion)

A. Medical History and Physical Examination including vital signs, height and weight. Performance status. Complete blood count (CBC) with differential and platelet count. Serum Chemistries: transaminases (AST, ALT), alkaline phosphatase, total and direct bilirubin, creatinine, albumin, serum creatinine To be performed at Day 0 and weekly during the treatment period.

B. EKG within 14 days of enrollment (baseline and prn).

C. CT scan, MRI and/or PET/CT scan at every 12 weeks.

Follow Up and Evaluation During and Post Intervention

During vector infusion and follow-up, the patient will be closely monitored for adverse events or changes in clinical status. The patient will be closely followed as an inpatient or outpatient during the entire study period and at regular intervals.

Stopping Rules

A. The NCI Common Toxicity Criteria (CT-CAE version 3.0) will be used to achieve consistency in response to drug/intervention toxicities. Toxicity will be graded on a 1 to 5 grading scale.

The patient will receive REXIN-G intravenously at a dose of 2×10¹¹ cfu per dose, five days a week, for 4 weeks. If there is <Grade I toxicity, may continue REXIN-G at a dose of 2×10¹¹ cfu 3 days a week for 8 more weeks. If the patient develops a Grade 3 or greater adverse event (CTCAE Vs 3.0) which appears to be related or possibly related to REXIN-G, the infusion will be held and the patient will be monitored until the toxicity resolves or the patient is stable. The infusion may be considered to be resumed if the toxicity is Grade 3 and resolved to Grade 1 or less within 24 hours. If the adverse event does not resolve within 72 hours, the study will be held until the data are discussed with the Food and Drug Administration (FDA) and a decision is made whether to continue or terminate the study.

All drug-related serious or unexpected adverse events will be reported immediately within 24 hours to the sponsor and the IRB, and to the FDA within 7 days of incident. All other adverse events will be reported to the FDA and IRB in annual report format and in the final study report.

For Grade III adverse events not related to vector infusions, the investigators will discuss the various options available. The appropriate action relative to the patient with a Grade III adverse event will be evaluated. If appropriate, a decision of whether the patient shall continue vector infusions will be made. In the event of death, permission to perform an autopsy will be requested.

The risks associated with retroviral vector infusion include development of replication competent retrovirus, vector neutralizing antibodies, vector integration in non-target organs. Acute toxicity may occur as outlined in the common toxicity criteria, from destruction of the tumor by the cytocidal REXIN-G vector or from unknown vector toxicity. All Grade III or IV toxicities, whether or not they are attributable to the study drugs, will be reported. In the event of death, an autopsy report will be submitted if a post-mortem examination was conducted.

### Example 25

### Intensification of REXIN-G with Hepatic Arterial Infusion

Patient is a 67 year-old Asian male, with adenocarcinoma of the tail of the pancreas, S/P distal pancreatectomy, pancreatico jejunal anastomosis, jejuno-jejunal downstream Rou-Y anastomosis, and splenectomy (Oct. 30, 2008). The histopathological findings of this T3N1 disease were consistent with intraductal (pancreatic duct) papillary adenocarcinoma that is epidermal growth factor negative but Kras positive. Post-operatively, the course was complicated by pancreatico-jejunal disruption, subphrenic abscess and fistula formation. These complications slowly improved with percutaneous subphrenic catheter drainage, and broad spectrum i.v. antibiotics. Adjuvant chemotherapy consisted of 6 cycles of gemcitabine and capecitabine.

REXIN-G Monotherapy: On Feb. 24, 2010, a follow-up CT scan showed recurrence of malignant tumor at the surgical site with metastases to the liver. The patient was then referred for consideration of REXIN-G monotherapy. Having failed standard therapy for pancreas cancer, the patient began REXIN-G therapy on Mar. 10, 2010, at 2×10e11 cfu/dose, i.v., 5 days a week for 12 weeks. A follow-up PET-CT scan on Apr. 7, 2010 confirmed a previously small suspicious liver lesion to be a definite hypermetabolic lesion. On Jun. 8, 2010, after the 3^{rd} cycle of REXIN-G was completed, the PET scan showed a mixed tumor response with (i) a dramatic decrease in size and metabolic activity at the left subphrenic area (primary site recurrence), (ii) increased sizes and metabolic activities in two liver lesions, and (iii) a complete absence of new lesions during the REXIN-G treatment.

Intensification of REXIN-G: To increase the regional concentrations of REXIN-G in the liver, the patient was referred to an Interventional Hepatologist for evaluation, looking into the possibility of Hepatic Arterial Infusion (HAI) of REXIN-G. At this time, a pre-procedural baseline ultrasound of the upper abdomen was performed.

Under local 2% Lidocaine anesthesia, a 5F femoral arterial sheath was inserted percutaneously into the right femoral artery and a 5F Terumo Yashiro was used with a co-axial 3F Terumo Progreat catheter to perform selective and superselective contrast examination of the superior mesenteric, celiac, common hepatic, hepatic proper, gastroduodenal, right hepatic, middle hepatic, and pancreaticoduodenal circulations. Anterior and oblique projections were taken, and a total volume of 110 ml of non-ionic contrast medium (Ultravist-Iopromide) was instilled.

Radiological Findings: Brisk hepatopetal visualization of the portal venous segments indicated no traces of collateral vessel formation. Hypovascular tumor nodules were seen in the medial segment of the right hepatic lobe with mild neovascularities and patchy tumor staining, revealing blood supplies from the right hepatic, middle hepatic, and pancreaticoduodenal arteries.

Dose-Dense Treatment with REXIN-G by HAI: Skillful and selective catheterization facilitated the infusion of 40 ml of REXIN-G (5×10e9 cfu/ml) sequentially at a rate of 4 ml/min into the pancreaticoduodenal (10 ml), right hepatic (10 ml), and middle hepatic (20 ml) artery supplies of the target lesions, respectively, in proportion to visual estimates of contribution of each vessel. The same infusions were repeated for 2 additional days with re-accessing of the same vessels.

Safety Analysis: No adverse events occurred during the HAI procedure, nor during the subsequent three days of HAI with REXIN-G. There was no nausea or vomiting, fever, bone marrow suppression, liver or kidney dysfunction noted either during or after HAI with REXIN-G.

The Tables below show the results of serum chemistry and hematology studies obtained before and after HAI of REXIN-G (2×10e11 cfu/dose)×3 days (Cumulative Dose: 6×10e11 cfu):

| Serum Chemistry | Before HAI | After HAI |
|---|---|---|
| Total Bilirubin | 0.74 | 0.78 |
| Direct Bilirubin | 0.52 | 0.55 |
| Indirect Bilirubin | 0.22 | 0.23 |
| Alk Phosphatase | 153.34 | 164.09 |
| AST | 30.46 | 22.43 |
| ALT | 23.35 | 22.15 |
| Creatinine | 1.32 | 1.31 |

| Hematology | Before HAI | After HAI |
|---|---|---|
| WBC | 5.8 | 4.0 |
| Hemoglobin | 11.00 | 12.00 |
| Hematocrit | 0.34 | 0.38 |
| Segs | 0.71 | 0.69 |
| Lymphs | 0.25 | 0.28 |
| Platelet Count | 199.00 | 265.00 |

Efficacy Analysis: Abdominal ultrasound performed before (Day 1) and after (Day 7) of the HAI with REXIN-G revealed a decrease in the sizes of the two hepatic lesions as follows:

Segment 4: 41% decrease in tumor volume

Before—2.6×2.1×2.5 (Tumor Volume 13.65 cc)

After—2.14×1.8×2.1 (Tumor Volume=8.09 cc)

Segment 5: 8% decrease in tumor volume

Before—3.6×3.3×3.6 (Tumor Volume=42.8 cc)

After—3.3×3.24×3.69 (Tumor Volume=39.45 cc)

CONCLUSIONS: These findings suggest that REXIN-G may be safely and effectively delivered both systemically, via intravenous infusion for general metastatic tumor control, and regionally via the hepatic artery for enhanced and expedient control of liver metastases. The plan for this patient going forward is the placement of a percutaneous ‘porta cath’ using a transaxillary approach to facilitate repeated dose-dense cycles of REXIN-G via Hepatic Artery Infusions, in addition to receiving continued systemic (intravenous) infusions of REXIN-G.

### Example 26

### Intensification of REXIN-G Treatment by Hepatic Arterial Infusion Plus Intravenous Infusion for Primary or Secondary (Metastatic) Liver Malignancies

Number of Patients, Investigators and Sites: Twenty to forty patients will be enrolled. This will be an open label, single arm, multisite study.

REXIN-G is a replication-incompetent, pathotropic (disease-seeking), tumor matrix (collagen)-targeted retrovector encoding an N-terminal deletion mutant of the cyclin G1 gene with potential antineoplastic activity (NCI Thesaurus C49082). REXIN-G nanoparticles exhibit a physiological surveillance function with an intrinsic affinity to bind to newly exposed extracellular matrix proteins found in cancerous lesions—based on the molecular engineering of a collagen-binding motif derived from von Willebrand coagulation factor (vWF) onto the retrovector's surface. Exploiting the natural collagen-targeting mechanism of vWF permits delivery of the retrovector selectively to primary tumors and metastatic sites where angiogenesis and collagen matrix exposure characteristically occur. The pathotropic nanoparticles carry a cytocidal ‘dominant negative’ cyclin G1 construct as the genetic payload, which has the ability to destroy or retard growth of tumor cells by disruption of tumor cell cyclin G1 activity, thus inducing apoptosis of tumor cells and the proliferative tumor-associated vasculature.

In preclinical proof-of-concept studies, REXIN-G, given intravenously, has been shown to concentrate selectively in cancerous lesions and to attenuate tumor growth in human xenograft models of metastatic cancer. In clinical studies, REXIN-G has been demonstrated to have significant anti-tumor activity in a number of solid tumor tissues, including breast, colon, lung, skin, muscle and bone, as well as pancreas cancer. Following on from initial Phase I safety studies and Phase I/II adaptive studies, REXIN-G was granted Orphan Drug Status by the U.S. FDA in 2008 for soft tissue sarcoma and osteosarcoma, in addition to pancreas cancer in 2003. Advanced Phase I/II clinical studies of REXIN-G for pancreatic cancer have shown that REXIN-G is well-tolerated with an excellent safety/toxicity profile and is associated with significant tumor regression and prolonged progression-free survival (by RECIST criteria), with a tentative indication that REXIN-G monotherapy may improves overall survival as well (Chawla et al. 2009). The Phase 4 study is designed to improve objective tumor responses without compromising safety of REXIN-G by combining regional delivery (via hepatic artery infusions for local control) and intravenous infusions (for systemic control) of REXIN-G for primary and secondary (metastatic) liver malignancies.

Objectives: Primary—To evaluate the efficacy of combination hepatic arterial infusion and intravenous infusion of REXIN-G in terms of objective tumor responses. Secondary—To evaluate the safety/toxicity of combination hepatic arterial infusion and intravenous infusion of REXIN-G

Study Design—The proposed Phase 4 study is designed as an open-label, single-arm, multicenter study of combination hepatic arterial infusion (for local control) and intravenous infusion (for systemic control) of REXIN-G treatment for primary or secondary (metastatic) liver malignancies.

Dosing and Conduct of Study: 20 to 40 patients will receive the REXIN-G via hepatic arterial infusion on Days 1-3 and Days 11-13 and REXIN-G intravenously, on Days 4-10, and Days 14-20. Stopping rules will be met if at any time, after 10 or more patients have had a full cycle of exposure to study drug, more than one third of patients in the course of a cycle have had grade 3-5 drug-related (possibly, probably or definitely related) toxicities (using CTCAEvs3). Epeius Biotechnologies Corporation, in consultation with the FDA, will make all final decisions regarding termination or continuation of the study.

# of Patients: 20-40; Vector Dose: 2×10e11 cfu; Maximum Volume: 40 ml

Primary Endpoint: Favorable objective tumor response in terms of complete or partial response or stabilization of disease by CT scan, MRI or Ultrasound.

Secondary Endpoint: Acceptable clinical toxicity profile by NIH-CT-CAE vs. 3

Inclusion Criteria: Patient is ≧18 years of age, either male or female; Patient has histology-proven primary or secondary (metastatic) liver malignancy; Patient is not part of any other experimental drug program; ECOG status 0-1 with life expectancy of 3 months; Patient has no evidence of active infection; Patient has no existing chronic condition (i.e., severe atherosclerosis, collagen-vascular disease, multiple sclerosis, recent MI or coagulopathy, cardiomyopathy, etc.) that would compromise successful adherence to the protocol; Patient has adequate hematologic and organ function, as determined by laboratory testing of blood and serum (as described further in the detailed protocol); Patient has NO ascites, pleural effusion, or pericardial effusion; Patient has the ability to understand and willingness to sign a written informed consent; Patients with measureable disease, i.e., at least 1 cm in diameter by spiral CT scan, MRI or ultrasound; Patients agree to use barrier contraception during vector infusion period and for 6 weeks after infusion.

Exclusion Criteria: Patient has any medical condition which would interfere with the conduct of the study; Patient is unable or unwilling to provide formal informed consent; Pregnant, or nursing women or individuals of either sex unwilling to use adequate contraception measures; Concomitant use of other chemotherapeutic or immunotherapeutic agents during the study period.

Monitoring for Safety: Infusion-related toxicity will be monitored medically by observation and vital signs during REXIN-G infusion and for the first hour after the infusion. Otherwise, all adverse event (AE) data during the study period will be reported/collected at each weekly visit and graded using common toxicity criteria (CTCAE v.3.0).

The Responsible Investigators will report all SAEs to the sponsor or the sponsor's designated representative within 24 hours of becoming aware of the SAE occurrence. SAEs will be reported in a timely manner to the FDA and IRB, consistent with existing regulations for expedited or special reporting. Information on relevant AEs will be disseminated between sites in a timely manner.

Monitoring for Efficacy: Tumors will be evaluated radiologically by CT scan, MRI or ultrasound at baseline, on Day 7 and Day 21. The patient's best response on therapy (based on RECIST criteria or Tumor Volume) will be captured. The number (proportion) of responders (CR+PR+SD) versus non-responders (PD) will be determined. The same statistical methods will be conducted for both the Intent-to-Treat (ITT) and the Modified Intention-to-Treat (mITT) populations. The ITT population will consist of all subjects, regardless of the treatment or amount of treatment actually received. The mITT population will be composed of all patients who have completed at least the 20-day treatment with of REXIN-G and had a tumor response evaluation by CT scan, MRI or ultrasound on Day 21. Tumor response evaluation will be done by site investigators and may be verified by an independent central site using blinded reviewer(s) at specified time points.

Endpoints: The Primary Endpoint will be a favorable objective tumor response (complete response, partial response or stable disease) in the majority of treated patients. The Secondary Endpoint will be acceptable clinical toxicity, with one-third or less of patients experiencing a Grade 3 or greater drug-related toxicity.

Exploratory Endpoints—Associations of tumor marker levels, tumor burden, and time from disease diagnosis with outcomes/endpoint listed above. The same statistical methods will be conducted for both the Intent-to-Treat (ITT) and the Modified Intention-to-Treat (mITT) populations.

Study Visits: Visits will be scheduled at screening and weekly for up to 21 days from start of REXIN-G treatment. Infusion visits will be considered unscheduled visits during which only vital signs will be routinely recorded. Tumor response evaluation will be obtained at Days 7 and 21. The end-of-study visit will be at 21 days. All patients who at end-of-study visit have at least one Grade 2 or higher AE or SAE will be followed for 30 days longer. Patients who complete the study period of 21 days will be placed in a follow-up group and contacted every 3 months to capture unexpected safety events and history of cancer disease progression and to ascertain survival for up to 15 years after study initiation.

Statistical Analysis: This Phase 4 study is expected to accrue up to 20-40 patients; it should take approximately 12 months to complete this trial and is exploratory in nature to gain insight into the potential benefit of an intensified treatment with HAI added to i.v. infusions of REXIN-G. Although this study will not be large enough to allow firm conclusions about safety or efficacy, it will provide preliminary data on safety and efficacy that will be useful in planning future studies. Demographic and baseline information (e.g., extent of prior therapy) on study patients will be tabulated. The following information will be reported for adverse events observed in the study: type (organ affected or laboratory determination, such as absolute neutrophil count), severity (by NCI Common Terminology Criteria for Adverse Events (CTCAE) Version 3.0 and most extreme abnormal values for laboratory determinations) and relatedness to study treatment.

Efficacy information will be summarized for each dose as the number and percentage in each of the categories PD, SD, PR, and CR. In addition, information will be reported for the following events: death from any cause, disease progression or death from any cause, and disease progression or death due to the underlying cancer. Patients will be followed for survival for 15 years. Response rates will be reported both as the percentage of eligible patients enrolled in the study (“intent-to-treat” or ITT analysis) and as the percentage of evaluable patients (i.e., eligible patients who finish the treatment course) (“as modified intent-to-treat” or mITT analysis); 95% confidence intervals for the response rates will be estimated. Survival and time to failure will be summarized with Kaplan-Meier plots.

### Example 27

### Protocol for Hepatic Arterial Infusion of REXIN-G

The following is a clinical protocol for the treatment of metastatic hepatic cancer.

Day 1-3 Admit patient; Obtain informed consent and waiver of hospital liability

Pre-treatment Studies: Abdominal CT Scan or MRI or Abdominal Ultrasound (one day before HAI); Chest X-ray and EKG (within 14 days); CBC, platelet count, Chem panel (BUN, Creatinine, AST, ALT, Alk Phos, Bilirubin); Electrolytes, PT, PTT, HIV, HBV, HCV, CEA; Daily CBC, platelet count, Chem panel (BUN, Creatinine, AST, ALT, Alk Phos, Bilirubin) Electrolytes, PT, PTT

Document patient eligibility for hepatic arterial infusion.

Schedule hepatic artery catheter placement with interventional radiologist.

Antibiotic prophylaxis: Imipenim (500 mg) IV over 15-30 min before procedure (and q 6 hrs×72 hrs). Note: Patients with a history of penicillin sensitivity will receive ceftazidime (2 grams) IV q 8 hr and metronidazole (500 mg) IV q 6 hrs.

Hepatic Artery Catheterization: Hepatic artery catheter placement per procedure by interventional radiologist

Follow interventional radiologist's heparin protocol for hepatic catheter placement.

REXIN-G Infusion through hepatic artery catheter:

Pre-medications: 30 min before infusion: Benadryl 25-50 mg p.o or i.v.; Hydrocortisone 50-100 mg IV. Discontinue heparin through hepatic artery catheter during REXIN-G infusion. Infuse 40 ml (2×10e11 cfu) of REXIN-G at a rate of 4 ml/min once a day through hepatic artery catheter for three days. Remove hepatic artery catheter.

If Hepatic Artery Catheter is kept in place for three days: Strict bed rest×72 hours while hepatic artery catheter is in place; May elevate head 45° Insert Foley catheter, I & O×72 hrs while hepatic artery catheter is in place.

Heparinization through Hepatic Artery Catheter: Infuse Heparin 2,000 Units/500 ml Normal Saline at 80 Units or 20 ml/hr through hepatic artery catheter×72 hrs to keep arterial line open.

Vital signs, lower extremity neuro and vascular checks q 15 min×4, then 1 half-hr×4, then q1 hr×72 hrs

PT and PTT q 12 hrs×72 hrs; Check for bleeding from groin area or abdominal pain.

Heparinization through Peripheral IV line: Heparin 25,000 Units/250 ml D5W at 800 Units/hr through peripheral IV×72 hrs. Adjust dose to maintain PTT within 1.5× normal; check for bleeding

Resume heparin through hepatic artery catheter after each REXIN-G infusion is completed.

Day 3: Discontinue heparinization after REXIN-G infusion is completed. Remove hepatic artery catheter by interventional radiologist.

Day 4-7 Infuse REXIN-G, 2×10e11 cfu, i.v. at 4 ml/min once a day for 4 days. Follow-up Abdominal CT Scan or MRI or Abdominal Ultrasound, CBC, platelet count, Chem panel (BUN, Creatinine, AST, ALT, Alk Phos, Bilirubin) Electrolytes, PT, PTT

Discharge patient if stable and if PT and PTT have returned to normal with no signs of bleeding.

The present invention is not to be limited in scope by the specific embodiments described herein. While preferred embodiments of the present invention have been shown and described herein, it will be obvious to those skilled in the art that such embodiments are provided by way of example only. Indeed, various modifications of the invention in addition to those described will become apparent to those skilled in the art from the foregoing description and accompanying figures. It is intended that the following claims define the scope of the invention and that methods and structures within the scope of these claims and their equivalents be covered thereby.

Various publications are cited herein, the disclosures of which are incorporated by reference in their entireties.

1.-56. (canceled)

57. A method of treating hepatic cancer in a subject in need thereof with a targeted therapeutic retroviral particle, the method comprising:
a) systemically administering a first therapeutic course of at least 1×10¹¹ cfu cumulative dose of a targeted therapeutic retroviral particle for at least three days;
b) administering via hepatic-arterial infusion a second therapeutic course of at least 1×10¹¹ cfu cumulative dose of a targeted therapeutic retroviral particle to the subject for at least three days;
c) monitoring the subject for improvement of cancer symptoms.

58. The method of claim 57, further comprising a third therapeutic course of at least 1×10¹¹ cfu of targeted therapeutic retroviral particles following step b).

59. The method of claim 57, wherein at least 1×10¹² cfu cumulative dose is administered as a first and/or second therapeutic course.

60. The method of claim 57, wherein at least 1×10¹³ cfu cumulative dose is administered as a first and/or second therapeutic course.

61. The method of claim 57, wherein the first and second therapeutic courses are administered sequentially.

62. The method of claim 57, wherein the first and second therapeutic courses are administered concurrently.

63. The method of claim 57, wherein the subject is allowed to rest 1 to 2 days between the first therapeutic course and second therapeutic course.

64. The method of claim 57, wherein the first therapeutic course comprises administration of the targeted therapeutic retroviral particles topically, intravenously, intra-arterially, intracolonically, intratracheally, intraperitoneally, intranasally, intravascularly, intrathecally, intracranially, intramarrowly, intrapleurally, intradermally, subcutaneously, intramuscularly, intraocularly, intraosseously and/or intrasynovially.

65. The method of claim 64, wherein the first therapeutic course comprises administration of the targeted therapeutic retroviral particles intravenously.

66. The method of claim 57, wherein the subject is a mammal.

67. The method of claim 57, wherein the subject is a human.

68. The method of claim 57, wherein the targeted therapeutic retroviral particles accumulate in the subject in areas of exposed collagen.

69. The method of claim 68, wherein the areas of exposed collagen include neoplastic lesions, areas of active angiogenesis, neoplastic lesions, areas of vascular injury, surgical sites, inflammatory sites and areas of tissue destruction.

70. The method of claim 57, wherein the targeted therapeutic retroviral particle is a retroviral vector having an envelope protein modified to contain a collagen binding domain, and encodes a therapeutic agent against the cancer.

71. The method of claim 70, wherein the retroviral vector is amphotropic.

72. The method of claim 70, wherein the therapeutic agent is a cyclin G1 mutant.

73. The method of claim 70, wherein the therapeutic agent is an N-terminal deletion mutant of cyclin G1.

74. The method of claim 73, wherein the N-terminal deletion mutant of cyclin G1 comprises from about amino acid 41 to 249 of human cyclin G1.

75. The method of claim 70, wherein the therapeutic agent is interleukin-2 (IL-2).

76. The method of claim 70, wherein the therapeutic agent is granulocyte macrophage-colony stimulating factor (GM-CSF).

77. The method of claim 70, wherein the therapeutic agent is thymidine kinase.

78. The method of claim 70, wherein the retroviral vector is produced by a method comprising:
(a) transiently transfecting a producer cell with:
a first plasmid comprising a nucleic acid sequence encoding the 4070A amphotropic envelope protein modified to contain a collagen binding domain, wherein the nucleic acid sequence is operably linked to a promoter;
a second plasmid comprising:
a nucleic acid sequence operably linked to a promoter, wherein the sequence encodes a viral gag-pol polypeptide,
a nucleic acid sequence operably linked to a promoter, wherein the sequence encodes a polypeptide that confers drug resistance on the producer cell,
an SV40 origin of replication;
a third plasmid comprising:
a heterologous nucleic acid sequence operably linked to a promoter, wherein the sequence encodes a diagnostic or therapeutic polypeptide,
5′ and 3′ long terminal repeat sequences (LTRs),
a Ψ retroviral packaging sequence,
a CMV promoter upstream of the 5′ LTR,
a nucleic acid sequence operably linked to a promoter, wherein the sequence encodes a polypeptide that confers drug resistance on the producer cell,
an SV40 origin of replication,
wherein the producer cell is a human cell that expresses SV40 large T antigen;
(b) culturing the producer cells of a) under conditions that allow targeted delivery vector production and release in to the supernatant of the culture;
(c) collecting the retroviral vectors.

79.-85. (canceled)

86. The method of claim 57, further comprising administering to the subject a chemotherapeutic agent, a biologic agent, or radiotherapy prior to, contemporaneously with, or subsequent to the administration of the therapeutic viral particles.

87. The method of claim 57, wherein the targeted therapeutic retroviral particles comprises a collagen binding domain comprising a peptide derived from the D2 domain of von Willebrand factor.

88-91. (canceled)

92. The method of claim 57, wherein abdominal CT scan, MRI, abdominal ultrasound, CBC, platelet count, Chem panel (BUN, Creatinine, AST, ALT, Alk Phos, Bilirubin), electrolytes, PT or PTT measurements are monitored in the subject for improvement of cancer symptoms.

93-99. (canceled)


SEQUENCE LISTING (7 sequences)

SEQ ID NO: 1 - protein, 16 aa, Artificial Sequence
  Description of Artificial Sequence Synthetic peptide
GHVGWREPSFMALSAA

SEQ ID NO: 2 - protein, 16 aa, Artificial Sequence
  Description of Artificial Sequence Synthetic peptide
GHVGWREPSFMAKSAA

SEQ ID NO: 3 - protein, 10 aa, Artificial Sequence
  Description of Artificial Sequence Synthetic peptide
WREPSFMALS

SEQ ID NO: 4 - protein, 6 aa, Unknown
  Description of Unknown Viral envelope sequence
GGWSHW

SEQ ID NO: 5 - DNA, 48 bp, Artificial Sequence
  Description of Artificial Sequence Synthetic oligonucleotide
ggacatgtaggatggagagaaccatcattcatggctctgtcagctgca

SEQ ID NO: 6 - DNA, 52 bp, Artificial Sequence
  Description of Artificial Sequence Synthetic primer
atgcggccgcccaccatggcgcgttcaacgctctcaaaaccccctcaagata

SEQ ID NO: 7 - DNA, 42 bp, Artificial Sequence
  Description of Artificial Sequence Synthetic primer
cctctagattatcatggctcgtactctatgggttttagctgg